(19) United States
(12) Patent Application Publication
(10) Pub. No.: US 2025/0051414 A1
(43) Pub. Date: Feb. 13, 2025

(54) ALTERING CYTOKINE SPECIFICITY THROUGH BINDING VALENCY

(71) Applicant: The Regents of the University of California, Oakland, CA (US)

(72) Inventor: Aaron S. MEYER, Los Angeles, CA (US)

(73) Assignee: The Regents of the University of California, Oakland, CA (US)

(21) Appl. No.: 18/570,542

(22) PCT Filed: Jun. 30, 2022
(86) PCT No.: PCT/US22/35711

§ 371 Date: Dec. 14, 2023

Related U.S. Application Data
(60) Provisional application No. 63/216,718, filed on Jun. 30, 2021.

(51) Int. Cl. C07K 14/55; A61K 38/00; A61P 37/06; C07K 14/54

(52) U.S. Cl. CPC C07K 14/55; A61P 37/06; C07K 14/54; C07K 14/5406; C07K 14/5418; C07K 14/5425; C07K 14/5443; A61K 38/00; C07K 2319/30

(57) ABSTRACT

The present disclosure provides a multivalent biomolecule comprising three or more covalently linked common γ-chain receptor cytokines (e.g., IL-2, IL-4, IL-7, IL-9, IL-15 or IL-21, or an immunologically active fragment thereof). In one embodiment, the common γ-chain receptor cytokines in the multivalent biomolecule are expressed as Fc fusion proteins with an IgG1 Fc. The multivalent biomolecule presented herein can be used to activate or suppress immune responses in a subject.

### CROSS-REFERENCE TO RELATED APPLICATION

This application claims priority to U.S. provisional patent application Ser. No. 63/216,718, filed Jun. 30, 2021, the entire contents of which is incorporated herein by reference.

### FIELD

The present disclosure is related in general to the field of cytokine signaling. In one embodiment, the present disclosure provides strategies for altering cytokine signaling through changes in binding valency.

### BACKGROUND

Originally identified as the third subunit of the high-affinity IL-2 receptor complex, the common γ-chain (γ_{c}) also acts as a non-redundant receptor subunit for a series of other cytokines, collectively known as γ_{c} family cytokines. Cytokines belonging to the common cytokine receptor gamma chain family include IL-2, IL-4, IL-7, IL-9, IL-15, and IL-21. Members of this family signal through receptor complexes that contain the common gamma chain subunit. This subunit associates with different cytokine-specific receptor subunits to form unique heterodimeric receptors for IL-4, IL-7, IL-9, and IL-21, or associates with both IL-2/IL-2Rβ and IL-2Rα or IL-15Rα to form heterotrimeric receptors for IL-2 or IL-15, respectively. Common gamma chain family cytokines generally activate three major signaling pathways that promote cellular survival and proliferation: the PI3K-Akt pathway, the RAS-MAPK pathway, and the JAK-STAT pathway.

Consistent with the involvement of Tc in diverse cytokine receptor complexes, the chain is expressed constitutively by multiple hematopoietic cell types, including macrophages and T, B and NK cells. Unlike most other cytokine receptors, Tc is thought to be constitutively expressed and functions only after the assembly of high-affinity cytokine receptor complexes.

Common gamma chain family cytokines serve as critical regulators of the development, survival, proliferation, differentiation and/or function of multiple immune cell types. These cytokines can have both unique and overlapping effects on different cell types, depending primarily on the expression patterns of the cytokines and their unique receptor subunits. Inactivating mutations in the common gamma chain family cytokines, their receptors, or a subset of intracellular signaling molecules involved in these pathways can lead to severe immune system defects. The most common form of severe combined immunodeficiency, X-linked SCID, is caused by mutations in the common cytokine receptor gamma chain subunit.

Many therapies, such as engineered antibodies, cytokines, and chimeric antigen receptors, aim to specifically expand, reduce, or alter the function of specific cells within the body. These strategies derive their specificity from the unique surface profiles of a target population. Specificity must be designed to overcome challenges from cell heterogeneity, subtle distinctions from off-target populations, and uniqueness that is only found through combinations of markers. These challenges make quantitative strategies for engineering cell specificity critical.

Cytokines that bind to the common γ-chain (γ_{c}) receptor, such as interleukin (IL)-2, 4, 7, 9, 15, and 21, are a critical hub in modulating both innate and adaptive immune responses. The cytokine family operates through a common theme of binding private receptors for each ligand before engaging the common γ_{c} receptor to induce signaling. A prominent phenotypic outcome of Tc receptor signaling is lymphoproliferation, and so the cytokines are often observed to be an endogenous or exogenous mechanism for altering the balance of immune cell types. This phenotype is observed most extremely from loss-of-function or reduced activity mutations in γ_{c} which subvert T and NK cell maturation. Disruptive mutations in private receptors can lead to more selective reductions in cell types such as regulatory T cells (T_{reg}s) with IL-2Rα or T cells with IL-7Rα. Conversely, activating mutations in these receptors, such as IL-7Rα, promote cancers such as B and T cell leukemias.

The importance of these cytokines to immune homeostasis and challenges in altering their signaling toward specific therapeutic goals have inspired a variety of engineered forms. Perhaps the most common approach has been to alter the receptor affinities of IL-2 to weaken its interaction with IL-2Rα, IL-2Rβ, or both receptors. IL-2Rα confers T_{reg}s with greater sensitivity toward IL-2, and so IL-2Rα affinity tunes the relative amount of signaling toward regulatory versus effector populations, while IL-2Rβ modulates the overall signaling potency. In most cases, the wild-type cytokine or mutein is fused to an IgG antibody to take advantage of FcRn-mediated recycling for extended half-life. Fc fusion has taken many forms, including orienting the cytokine in an N-terminal or C-terminal orientation, including one or two cytokines per IgG, and including or excluding Fc effector functions. The potential design space for these molecules quickly becomes experimentally intractable without consistent design principles.

Thus, there is a need to further evaluate the signaling effects of specific engineered cytokine alterations, e.g., affinity-altering mutations and Fc-fusion formats, in the design of strategies to alter signaling toward specific therapeutic goals.

### SUMMARY

In one aspect, the present disclosure provides a multivalent biomolecule comprising three or more covalently linked common γ-chain receptor cytokines (e.g., IL-2, IL-4, IL-7, IL-9, IL-15 or IL-21, or an immunologically active fragment thereof). In one embodiment, the common 7-chain receptor cytokines in the multivalent biomolecule are expressed as Fc fusion proteins with an IgG1 Fc, e.g., the common γ-chain receptor cytokines or immunologically active fragment thereof are expressed as Fc fusion proteins with human IgG1 Fc. In another embodiment, the common γ-chain receptor cytokines, or immunologically active fragments thereof, are multimerized and expressed as a single-chain polypeptide.

In one aspect, there is provided a method for modulating the immune system of a subject, comprising administering to a subject in need thereof the multivalent biomolecule disclosed herein. In one embodiment, the multivalent biomolecule is used to activate immune responses in the subject. In another embodiment, the multivalent biomolecule is used to suppress immune responses in the subject.

In some embodiments, a multivalent biomolecule is provided comprising three or more covalently linked common γ-chain receptor cytokines. In some embodiments, the common γ-chain receptor cytokines are IL-2, IL-4, IL-7, IL-9, IL-15 or IL-21, or immunologically active fragments thereof.

In some embodiments, the multivalent biomolecule comprises at least 3, at least 4, at least 5, at least 6, at least 7, or at least 8 common γ-chain receptor cytokines or immunologically active fragments thereof. In some embodiments, the multivalent biomolecule comprises 3, 4, 5, 6, 7 or 8 common γ-chain receptor cytokines or immunologically active fragments thereof.

In some embodiments of the multivalent biomolecule, the common γ-chain receptor cytokines are expressed as Fc fusion proteins of the common γ-chain receptor cytokines or immunologically active fragments thereof, with Fc. In some embodiments, the Fc is human IgG1 Fc.

In some embodiments of the multivalent biomolecule, the fusion protein comprises the common γ-chain receptor cytokines or immunologically active fragments thereof fused to the N- or C-terminus of Fc, e.g., human IgG1 Fc. In some embodiments, the common γ-chain receptor cytokines or immunologically active fragments thereof are fused to the N- or C-terminus of Fc, e.g., human IgG1 Fc, through a (G₄S)4 (SEQ IS NO:13) linker.

In some embodiments, the sequence GLNDIFEAQKIEWHE (SEQ ID NO:3) is fused to the terminus of the Fc, e.g., human IgG1 Fc, and fused to the common γ-chain receptor cytokines or immunologically active fragments thereof.

In some embodiments, the multivalent biomolecule comprises three or more SEQ ID NO:1, 12, 14 or 15 or 26-33.

In some embodiments, the multivalent cytokine is any one of SEQ ID NOs:2, 4-8, 10, 12, 17-25 or 34-75. In some embodiments, the multivalent cytokine comprises a multimer of any one of SEQ ID NOs: 9, 10, 58, 60, 11, 74, 62, 64, 66, 68, 70, 72 or 74, or any combination thereof.

In some embodiments, a nucleic acid is provided encoding a multivalent cytokine disclosed herein. In some embodiments, a nucleic acid is provided encoding any one of SEQ ID NOs:2, 4-8, 10, 12, 17-22, 34-57, 58, 60, 62, 64, 66, 68, 70, 72 or 74. In some embodiments, a vector is provided comprising a nucleic acid encoding a multivalent cytokine disclosed herein. In some embodiments, a vector is provided comprising a nucleic acid encoding any one of SEQ ID NOs:2, 4-8, 10, 12, 17-22, 34-57, 58, 60, 62, 64, 66, 68, 70, 72 or 74.

In some embodiments of the multivalent biomolecule, the common γ-chain receptor cytokines, or immunologically active fragments thereof, are multimerized and expressed as a single-chain polypeptide. In some embodiments, at least one common γ-chain receptor cytokine, or immunologically active fragment thereof, comprises a signal sequence.

In some embodiments of the multivalent biomolecule, the affinity for the cognate γ-chain receptor is at least 2-fold lower, but with equal or greater avidity, than that of the same common γ-chain receptor cytokine in monomeric form. In some embodiments of the multivalent biomolecule, as compared to biomolecule comprising said γ-chain receptor cytokine in monomeric or dimeric form, said multivalent biomolecule has increased selective binding to cells expressing high level of cognate γ-chain receptor. In some embodiments the multivalent biomolecule produces an altered dynamic response as compared to a biomolecule comprising the γ-chain receptor cytokine in monomeric or dimeric form, the altered dynamic response is selected from among altered pharmacokinetics, altered signaling, altered intracellular degradation, altered in vivo half-life, or any combination thereof.

In some embodiments, the method is provided for modulating the immune system of a subject, comprising administering to a subject in need thereof the multivalent biomolecule of claim 1. In some embodiments, the modulating is activating immune responses in the subject, and the multivalent biomolecule comprises a cytokine selected from IL-2, IL-4, IL-7, IL-9, IL-15 or IL-21.

In some embodiments, the multivalent biomolecule is used for treating cancer.

In some embodiments, the modulating is suppressing immune responses in the subject, and the multivalent biomolecule comprises a cytokine selected from IL-2, IL-4, IL-7, IL-9, IL-15 or IL-21. In some embodiments, the multivalent biomolecule is used for treating an autoimmune disease or preventing transplant rejection. In some embodiments, the autoimmune disease is systemic lupus erythematosus.

In some embodiments, the methods comprising administering a multivalent cytokine of any one of SEQ ID NOs:2, 4-8, 10, 12, 17-25 or 34-75. In some embodiments, the multivalent cytokine comprises a multimer of any one of SEQ ID NOs: 9, 10, 58, 60, 11, 74, 62, 64, 66, 68, 70 or 72, or any combination thereof.

In some embodiments, a pharmaceutical composition is provided comprising a multivalent cytokine disclosed herein. In some embodiments, a pharmaceutical composition comprising a multivalent cytokine comprising three or more covalently linked common γ-chain receptor cytokines. In some embodiments, a pharmaceutical composition is provided comprising SEQ ID NOs:2, 4-8, 10, 12, 17-25 or 34-75. In some embodiments, a pharmaceutical composition is provided wherein the multivalent cytokine comprises a multimer of any of the Fc-containing polypeptides disclosed herein, e.g., SEQ ID NOs: 9, 10, 58, 60, 11, 74, 62, 64, 66, 68, 70, 72 or 74, or any combination thereof. These and other aspects of the invention will be appreciated from the ensuing descriptions of the figures and detailed description of the invention.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-1O demonstrate that systematic profiling of IL-2 muteins reveals determinants of response. FIG. 1A: Schematic of affinity and Fc-fused mono- and bivalent muteins. FIG. 1B: IL2Rα and IL2Rβ affinities of each IL-2 variant. FIG. 1C: Heatmap of phosphorylated STAT5 measurements for each cell type, time point, ligand, and concentration. pSTAT5 measurements are normalized for each cell type. FIGS. 1D-1O: STAT5 phosphorylation response curves for immune cells stimulated with select IL-2 muteins. Time points and cell types are indicated in subplot titles.

FIGS. 2A-2H demonstrate pSTAT5 response varies in a cell type- and treatment-specific manner. FIG. 2A: Scores and loadings plot of pSTAT5 signaling data as calculated using principal components analysis. FIG. 2B: Schematic representation of non-negative canonical polyadic (CP) decomposition. Experimental pSTAT5 measurements are arranged in a tensor according to the duration of treatment, ligand used, cytokine concentration, and cell type. CP decomposition then helps to visualize this space. FIG. 2C: Percent variance reconstructed (R2X) versus the number of components used. FIG. 2D: Component values for each IL-2 form. FIG. 2E: Component values representing the effect of IL-2 concentration. FIG. 2F: Component values representing cell type specificity. FIG. 2G: Component values for the effect of treatment duration. FIG. 2H: Sum of Component 1 and 3 weights vs. Component 2 weight for each monovalent and bivalent ligand.

FIGS. 3A-3K demonstrate responses are predicted by a simple multivalent binding model. All accuracies are calculated as a Pearson's correlation R² score for experimental cytokine responses at the 30 minute and one hour time points. FIG. 3A: A simplified cartoon of the model. Initial association of multivalent ligands proceeds according to monovalent affinity, and subsequent binding events proceed with that affinity scaled by the K*ₓ parameter. FIG. 3B: Experimentally measured versus predicted pSTAT5 responses for all cell types, ligands, and concentrations. Each point represents a single experimental measurement (N=1). FIGS. 3C-3D: Model's accuracy subset by cell type (FIG. 3C) and ligand (FIG. 3D) for all mono- and bivalent IL-2 muteins. FIG. 3E: Model's accuracy when predicting the responses of all cell types to bivalent ligands either correctly as dimers, or as IL-2 monomers. FIGS. 3F-3G: Model's accuracy subset by concentration (FIG. 3F) for all ligands and time (FIG. 3G) for all ligands, concentrations, and cell types. FIGS. 3H-3I: Model-predicted pSTAT for T_{reg}s (FIG. 3H) and NK cells (FIG. 3I) in response to mono- and bivalent IL-2 ligands with 10 nM IL2Rβ K_{D}. FIGS. 3J-3K: Predicted number of active signaling complexes formed on cells with 1000 IL2Rβ receptors and varying numbers of IL2Rα for ligands with affinities of 10 nM K_{D} for IL2Rβ and either 1 nM (FIG. 3J) or 10 nM (FIG. 3K) K_{D} for IL2Rα.

FIGS. 4A-4F demonstrate multivalency with coordinate affinity adjustments can enhance selectivity. Affinities were allowed to vary between K_{D}S of 10 pM and 1 μM while K*ₓ was fixed at its fitting optimum. All optimizations were performed using a concentration of 1 nM. Selectivity was calculated as the ratio of predicted pSTAT5 in target cells to the mean pSTAT5 predicted in off-target cells. FIGS. 4A, 4C, 4E: Signaling response of T_{reg}, NK cells, and Tₕₑₗₚₑᵣ cells predicted for ligand of optimal selectivity at different valency. Response predictions were normalized to each population's response for the monovalent case. Selectivity for T_{reg}s and NK cells were derived from IL-2 muteins, and selectivity for TₕₑₗₚₑᵣS was calculated using IL-7 muteins. FIGS. 4B, 4D, 4F: Optimal receptor-ligand dissociation constants for each ligand optimized for selectivity. Mutein affinity for IL-2Rα and IL-2Rβ/γ_{c} was allowed to vary for IL-2 muteins, and affinity for IL-7Rα was allowed to vary for IL-7 muteins.

FIGS. 5A-5J demonstrate receptor quantification and gating of PBMC-derived immune cell types. FIGS. 5A-5B: Gating for fixed Tₕₑₗₚₑᵣ and T_{reg} cells during pSTAT5 quantification.

FIGS. 5C-5D: Fixed CD8+ T cell and NK cell gating. FIGS. 5E-5F: Gating for live Tₕₑₗₚₑᵣ and T_{reg} cells during receptor quantification. FIG. 5G: Live NK cell gating. FIG. 5H: Live CD8⁺ cell gating. FIG. 5I: Receptor quantification for each cell type. FIG. 5J: IL-2Rα and IL-2Rβ abundances on IL-2Rα high and low T_{reg} and Tₕₑₗₚₑᵣ populations. Cells were binned using three evenly logarithmically spaced bins between 5^{th} and 95^{th} percentile of IL-2Rα abundance.

FIGS. 6 A-B shows a full panel of predicted versus actual immune cell type responses to monomeric and dimeric IL-2 muteins. Dots represent flow cytometry measurements and lines represent pSTAT response predicted by model. Experimental pSTAT measurements are shown for 0.5- and 1-hour time points. Predictions and experiments are shown for T_{reg}s, Tₕₑₗₚₑᵣs, NK and CD8 cells. Each point is representative of one experimental output (N=1). Shaded regions are indicative of standard error prediction when scalar factor converting between signaling complexes and MFI was fit to multiple experiments.

FIG. 7A-B shows a full panel of predicted versus actual IL-2Rα high medium, and low T_{reg} and Tₕₑₗₚₑᵣ responses to monomeric and dimeric IL-2 muteins. Dots represent flow cytometry measurements and lines represent pSTAT response predicted by model. Experimental pSTAT measurements are shown for 0.5- and 1-hour time points. Predictions and experiments are shown for T_{reg}s, Tₕₑₗₚₑᵣs which have been binned by their IL-2Rα abundances. Each point is representative of one experimental output (N=1). Shaded regions are indicative of standard error prediction when scalar factor converting between signaling complexes and MFI was fit to multiple experiments.

FIGS. 8A-8C present cell populations that are defined by quantitative receptor abundance differences. FIG. 8A: Measured receptor abundance for ten peripheral blood mononuclear cell (PBMC)-derived subpopulations. Points and error bars show geometric mean and standard deviation respectively (N=4). Error bars for some points are too small to display. FIG. 8B: Schematic of full receptor profiling data. For each point in FIG. 8A, the distribution of receptor abundances was quantified. FIG. 8C: Example data from dose-response profiling for IL-2/-15. Note that, unlike in FIG. 8A, the cells here were fixed and therefore provide total, not surface, measurements of IL-2Rα. Cells are distinguished based upon canonical markers. pSTAT5 indicates phosphorylated Signal Transducer and Activator of Transcription 5, a marker of IL-2 response.

FIG. 9 shows multivalent binding can preferentially target cells based on receptor expression. Model predictions using a simple, single receptor multivalent binding model. Ligand concentrations are adjusted to result in identical binding at 10,000 receptors per cell.

FIG. 10 shows altered specificity of IL-2-Fc conjugates matches predicted valency effects. Monovalent Fc conjugate data comes from a recent paper (Farhat et al, Modeling cell-specific dynamics and regulation of the common gamma chain cytokines, Cell Reports, 2021 April; 35(4):109044). Bivalent Fc conjugate is from the same study. As expected, multivalent interaction preferentially targets cells with higher expression of the high-affinity receptor IL-2Rα. Helper T cells also express IL-2Rα, but at moderately lower levels (see FIG. 8).

FIG. 11 A-G demonstrates greater T_{reg} selectivity derived from a tetravalent Fc fusion design. (a-d) Responses of human (a) T_{reg}, (b) Tₕₑₗₚₑᵣ, (c) NK, and (d) CD8+ cells, measured by STAT5 phosphorylation, in response to varying dosages of R38Q/H16N in monovalent, bivalent, and tetravalent form. Cells were stimulated with cytokine for 30 minutes. (e-g) Ratio of STAT5 phosphorylation in T_{reg}s to (e) Tₕₑₗₚₑᵣs, (f) NK cells, and (g) CD8+ cells at varying dosages for R38Q/H16N in monovalent, bivalent, and tetravalent form.

### DETAILED DESCRIPTION

The common γ-chain (γ_{c}) receptor cytokines, such as interleukin (IL)-2, 4, 7, 9, 15, and 21, are integral for modulating both innate and adaptive immune responses. The common γ-chain receptor cytokines are promising immune therapies due to their central role in coordinating the proliferation and activity of various immune cell populations. One of these cytokines, interleukin (IL)-2, has potential as a therapy in autoimmunity but is limited in effectiveness by its modest specificity toward regulatory T cells (T_{regs}). IL-2 muteins with altered receptor-ligand binding kinetics can improve the cell type selectivity of the signaling response. Furthermore, therapeutic ligands are often made dimeric as antibody Fc fusions to confer desirable pharmacokinetic benefits, with unexplored signaling consequences. The therapeutic potential and complexity of this cytokine family make computational models especially valuable for rational engineering. IL-2 is an approved, effective therapy for metastatic melanoma, and the antitumor effects of IL-2 and IL-15 have been explored in combination with other treatments. To address the limitations of natural ligands, engineered proteins have been produced with potentially beneficial properties. For example, mutants skewed toward IL-2Rα over IL-2Rβ binding selectively expand T_{reg} populations over cytotoxic T cells and NK cells as compared to native IL-2. Nonetheless, understanding these cytokines' regulation is stymied by their complex binding and activation mechanism. Any intervention imparts effects across multiple distinct cell populations, with each population having a unique response defined by its receptor expression.

In one embodiment, the disclosure presented herein systematically profiled the signaling responses to a wide variety of wild type and mutein IL-2 molecules in various Fc fusion configurations. A tensor-structured dimensionality reduction scheme was used to decompose the responses of each cell population to each ligand over a range of time points and cytokine concentrations. It was found that dimeric muteins are uniquely specific for T_{regs} at intermediate ligand concentrations, a desirable quality for immunosuppressive drugs. To dissect the mechanism of enhanced T_{reg} specificity in dimeric ligands, signaling response was compared across all treatments to a simple, two-step multivalent binding model. The model was able to predict cellular responses with high accuracy. Bivalent Fc fusions display enhanced specificity and potency for T_{reg}s through avidity effects toward IL-2Rα, and this enhancement is distinct from what was achieved by mutein affinity changes. The model presented herein can further be utilized to identify the potential benefits conferred by valency engineering as an additional mechanism for cytokines with optimized therapeutic benefits. In total, these findings represent a comprehensive analysis of how ligand properties, and their consequent effects on surface receptor-ligand interactions, translate to selective activation of immune cell populations. It also identifies a new route toward engineering even more selective therapeutic cytokines.

The present disclosure systematically evaluated the signaling specificity effects of engineered cytokine alterations, including affinity-altering mutations and Fc-fusion formats. It was found that the effect of bivalency can be fully explained by altered binding selectivity toward cells based on their receptor abundances. The signaling specificity of all muteins and Fc-formats match well with a multivalent binding model, both between cell types and across cell-to-cell variation within a cell type. Finally, it is believed that cytokine valency is an unexplored axis for further enhancing selective signaling responses and that many opportunities for using multivalency engineering exist within the γ_{c} cytokine family.

In one embodiment, the present disclosure provides a multivalent biomolecule comprising three or more covalently linked common γ-chain receptor cytokines. In another embodiment, the present disclosure provides a multivalent biomolecule comprising three or more covalently linked immunologically active fragments of common γ-chain receptor cytokines. Immunologically active fragments of common γ-chain receptor cytokines can be readily identified by one of ordinary skill in the art. Cytokines that are members of the common γ-chain receptor cytokine family are well-known in the art. Examples of common γ-chain receptor cytokines include, but are not limited to, IL-2, IL-4, IL-7, IL-9, IL-15 and IL-21. One of ordinary skill in the art would reasonably recognize that the principles and methodologies disclosed herein are applicable not only to IL-2, but also to other members of the common γ-chain receptor cytokine family, both currently known or discovered in the future.

In one embodiment, the multivalent biomolecule disclosed herein comprises at least 3, at least 4, at least 5 or at least 6 common γ-chain receptor cytokines or immunologically active fragments thereof. In one embodiment, the multivalent biomolecule disclosed herein comprises 3, 4, 5 or 6 common γ-chain receptor cytokines or immunologically active fragments thereof.

In one embodiment, the multivalent biomolecule disclosed herein comprises common 7-chain receptor cytokines or immunologically active fragments thereof that are expressed as Fc fusion proteins with a human IgG Fc, such as a Fc from IgG1, IgG2, IgG3 or IgG4. In one embodiment, the common γ-chain receptor cytokine or immunologically active fragment thereof is fused to the N-terminus of human IgG1 Fc. In another embodiment, the common γ-chain receptor cytokine or immunologically active fragment thereof is fused to the C-terminus of human IgG1 Fc. One of ordinary skill in the art would readily use generally known techniques to construct the Fc fusion proteins disclosed herein. In one embodiment, the common γ-chain receptor cytokine or immunologically active fragment thereof is fused to the N- or the C-terminus of human IgG1 Fc through a linker. Linkers useful for making the Fc fusion proteins include, but are not limited to, (G₄S)₄ and other generally known linkers. In one embodiment, a sequence (e.g., GLNDIFEAQKIEWHE [SEQ ID NO:3]) is fused to the terminus of the human IgG1 Fc that is not fused to the common γ-chain receptor cytokine or immunologically active fragment thereof. Thus, in one embodiment, the N-terminus of the Fc is fused to the common γ-chain receptor cytokine or immunologically active fragment thereof, optionally through a linker, and the C-terminus of the Fc is fused with GLNDIFEAQKIEWHE (SEQ ID NO:3). In one embodiment, the C-terminus of the Fc is fused to the common γ-chain receptor cytokine or immunologically active fragment thereof, optionally through a linker, and the N-terminus of the Fc is fused with GLNDIFEAQKIEWHE (SEQ ID NO:3).

In some embodiments, a multimer is provided of any of the polypeptides described herein comprising a Fc, such as but not limited to SEQ ID NOs:9, 10, 11, 58, 60, 74, 62, 64, 66, 68, 70, 72 or 74, or any combination thereof. In some embodiments the multimer is a dimer. In some embodiments the multimer is a trimer. In some embodiments the multimer is a homodimer, such as a dimer of two SEQ ID NO:9 (forming tetravalent SEQ ID NO:23), a dimer of two SEQ ID NO:10 (forming octavalent SEQ ID NO:24), a dimer of two SEQ ID NO:11 (forming tetravalent SEQ ID NO:25), a dimer of two SEQ ID NO:74 (forming octavalent SEQ ID NO:75), a dimer of two SEQ ID NO:58 (forming tetravalent SEQ ID NO:59), a dimer of two SEQ ID NO:60 (forming octavalent SEQ ID NO:61), a dimer of two SEQ ID NO:62 (forming tetravalent SEQ ID NO:63), a dimer of two SEQ ID NO:64 (forming octavalent SEQ ID NO:65), a dimer of two SEQ ID NO:66 (forming tetravalent SEQ ID NO:67), a dimer of two SEQ ID NO:68 (forming octavalent SEQ ID NO:69), a dimer of two SEQ ID NO:70 (forming tetravalent SEQ ID NO:71), a dimer of two SEQ ID NO:72 (forming octavalent SEQ ID NO:73).

In other embodiments, the dimer is a heterodimer comprising two different Fc containing polypeptides disclosed herein. Non-limiting examples include a multivalent heterodimer of IL-2 and IL-7, such as comprising SEQ ID NO:9 and SEQ ID NO:11, or comprising SEQ ID NO:9 and SEQ ID NO:74, or comprising SEQ ID NO: 10 and SEQ ID NO:11, or comprising SEQ ID NO:10 and SEQ ID NO:74. In other embodiments, similar heteromeric multivalent combinations of other cytokines from among IL-2, IL-4, IL-7, IL-9, IL-15 or IL-21 are provided.

In one embodiment, the multivalent biomolecule disclosed herein comprises the sequence of SEQ ID NO:2 (tetravalent IL-2).

In one embodiment, the common γ-chain receptor cytokines, or immunologically active fragments thereof, are multimerized and expressed as a single-chain polypeptide. Single-chain polypeptides containing multimerized subunits can be generated following standard molecular biology techniques. For example, multiple units of the common γ-chain receptor cytokines (or immunologically active fragments thereof) can be multimerized and covalently coupled via linkers generally known in the art. Alternatively, the multimerized subunits can be expressed as a repeating peptide chain. In one embodiment, one or more of the multimerized common γ-chain receptor cytokines, or immunologically active fragments thereof, is/are conjugated to an Fc for improved in vivo half-life.

In one embodiment, the present multivalent biomolecule comprising common γ-chain receptor cytokines, or immunologically active fragments thereof, has lowered affinity for the cognate γ-chain family receptor as compared to the same common γ-chain family receptor cytokine in monomeric form. In one embodiment, the affinity is lowered at least 2-fold. However, the present multivalent biomolecule comprising common γ-chain receptor cytokines, or immunologically active fragments thereof, exhibits equal or greater avidity for the cognate γ-chain family receptor as compared to the same common γ-chain family receptor cytokine in monomeric form.

In one embodiment, when compared to a biomolecule comprising the same γ-chain receptor cytokine in monomeric or dimeric form, the present multivalent biomolecule has increased selective binding to cells expressing a high level of the cognate γ-chain receptor.

In one embodiment, the multivalent biomolecule disclosed herein produces an altered dynamic response as compared to a biomolecule comprising the same γ-chain receptor cytokine in monomeric or dimeric form. Examples of the altered dynamic responses include, but are not limited to, altered pharmacokinetics, altered signaling, altered intracellular degradation, or altered in vivo half-life, or any combination thereof.

In one embodiment, the present disclosure provides a method for modulating the immune system of a subject, comprising administering to a subject in need thereof the multivalent biomolecule disclosed herein. In one embodiment, the multivalent biomolecule comprises IL-2, IL-4, IL-7, IL-9, IL-15 or IL-21, and the method is used to activate immune responses in the subject. In another embodiment, the multivalent biomolecule comprises IL-2, IL-4, IL-7, IL-9, IL-15 or IL-21, and the method is used to suppress immune responses in the subject. In one embodiment, the method can be used to treat cancer in the subject. In another embodiment, the method can be used to treat an autoimmune disease (e.g., systemic lupus erythematosus) or prevent transplant rejection in the subject. Such uses are non-limiting examples of the therapeutic utilities of the multivalent cytokines disclosed herein.

Examples of cancer include, but are not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoietic cancer, Non-Hodgkin lymphoma, skin cancer, basal-cell carcinoma, melanoma, small cell lung cancer, non-small cell lung cancer, or any combination thereof.

Examples of autoimmune disease include, but are not limited to, achalasia, amyloidosis, ankylosing spondylitis, antiphospholipid syndrome, arthritis, autoimmune angioedema, autoimmune encephalomyelitis, autoimmune hepatitis, autoimmune myocarditis, autoimmune oophoritis, autoimmune orchitis, autoimmune pancreatitis, autoimmune retinopathy, autoimmune urticaria, Behcet's disease, celiac disease, chagas disease, chronic inflammatory demyelinating polyneuropathy, Cogan's syndrome, congenital heart block, Crohn's disease, dermatitis, dermatomyositis, discoid lupus, Dressler's syndrome, endometriosis, fibromyalgia, fibrosing alveolitis, granulomatosis with polyangiitis, Graves' disease, Guillain-Barre syndrome, herpes gestationis, immune thrombocytopenic purpura, interstitial cystitis, juvenile arthritis, juvenile diabetes (type 1 diabetes), juvenile myositis, Kawasaki disease, Lambert-Eaton syndrome, lichen planus, lupus, Lyme disease, multiple sclerosis, myasthenia gravis, myositis, neonatal lupus, neutropenia, palindromic rheumatism, peripheral neuropathy, polyarteritis nodosa, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, reactive arthritis, retroperitoneal fibrosis, rheumatic fever, rheumatoid arthritis, sarcoidosis, Schmidt syndrome, scleritis, scleroderma, Sjögren's syndrome, thrombocytopenic purpura, type 1 diabetes, ulcerative colitis, uveitis, vasculitis, and vitiligo.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “treating”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition, or those in which the disease or condition is to be treated or prevented.

As used herein, “modulating” refers to “stimulating” or “inhibiting” an activity of a molecular target or pathway. For example, a composition modulates the activity of a molecular target or pathway if it stimulates or inhibits the activity of the molecular target or pathway by at least 10%, by at least about 20%, by at least about 25%, by at least about 30%, by at least about 40%, by at least about 50%, by at least about 60%, by at least about 70%, by at least about 75%, by at least about 80%, by at least about 90%, by at least about 95%, by at least about 98%, or by about 99% or more relative to the activity of the molecular target or pathway under the same conditions but lacking only the presence of the composition. In another example, a composition modulates the activity of a molecular target or pathway if it stimulates or inhibits the activity of the molecular target or pathway by at least 2-fold, at least 5-fold, at least 10-fold, at least 20-fold, at least 50-fold, at least 100-fold relative to the activity of the molecular target or pathway under the same conditions but lacking only the presence of the composition. The activity of a molecular target or pathway may be measured by any reproducible means. The activity of a molecular target or pathway may be measured in vitro or in vivo. For example, the activity of a molecular target or pathway may be measured in vitro or in vivo by an appropriate assay known in the art measuring the activity. Control samples can be assigned a relative activity value of 100%.

The multivalent cytokines disclosed herein are readily manufacturable using methods known in the art. For single-chain polypeptide expression, methods for cellular and acellular expression systems are known in the art, and methods for scale-up, preparation and purification of recombinant proteins for clinical use are well established. Methods for dimerization or oligomerization of the polypeptides described herein, using bifunctional cross-linking agents, or disulfide crosslinking of Fc portions of polypeptides described herein, are also known in the art. By way of non-limiting example, Fc regions of human IgG1 dimerize by disulfide formation at cysteines 109 and 112 (numbering based on the full heavy chain polypeptide, gene IGHG1). In the Fc sequence of SEQ ID NO:16, cysteines at positions 6 and 9 are involved in dimerization.

Other IgG isotypes form interchain disulfide cross-links at positions well known in the art.

Thus, in some embodiments, a nucleic acid is provided encoding a multivalent cytokine disclosed herein. In some embodiments, a nucleic acid is provided encoding any one of SEQ ID NOs:2, 4-8, 10, 12, 17-22, 34-57, 58, 60, 62, 64, 66, 68, 70, 72 or 74. In some embodiments, a vector is provided comprising a nucleic acid encoding a multivalent cytokine disclosed herein. In some embodiments, a vector is provided comprising a nucleic acid encoding any one of SEQ ID NOs:2, 4-8, 10, 12, 17-22, 34-57, 58, 60, 62, 64, 66, 68, 70, 72 or 74. Such nucleic acids and/or vectors are useful for preparing the multivalent cytokines disclosed herein, such as the single-chain polypeptides comprising multiple cytokines sequences, or the Fc constructs comprising multiple cytokine sequences described herein, that may then be dimerized.

Pharmaceutical compositions suitable for use in the methods disclosed herein include compositions wherein the active ingredients are contained in an amount effective to achieve the intended purpose. In one embodiment, a therapeutically effective amount means an amount of active ingredients effective to prevent, alleviate or ameliorate symptoms of disease (e.g., cancer, auto-immune disease) or prolong the quality of life or survival of the subject being treated. Determination of a therapeutically effective amount is well within the capability of those skilled in the art. For example, for treatment of cancer to include effector T cells, a dosing regimen based on prior studies with IL-2 indicate a dose of 600,000 IU (0.037 mg) IL-2 per kg, administered IV three times a day for 14 doses, followed by a 9 day rest period and another 14 doses. The relative efficacy of the multivalent cytokines disclosed herein compared to, e.g., IL-2, will be factored into the dose and dosing regimen calculations for this or other indications. For treatment of autoimmune disease, a lower dose of IL-2 therapy is known in the art to be effective; the dose of a multivalent cytokine disclosed here will be further adjusted based on the potency of the multivalent cytokine compared to readily-obtainable comparative data on monovalent cytokines. In one non-limiting example, a dose of multivalent IL-2 with potency equivalent to 1 million IU (0.062 mg) IL-2 given IV per day for 5 days, then once every 2 weeks for 6 months, is provided, the equivalent based on the efficacy of the multivalent cytokines disclosed herein. As described herein, the multivalent cytokines described herein will provide increased potency at equivalent or lower doses than monovalent cytokines currently on the market or in development.

Pharmaceutical compositions may comprise excipients, vehicles, diluents, carriers, and/or any other components to aid in the formulation, storage, aliquoting, vialing, sterilizing, packaging, distribution and/or administration of the multivalent cytokine to a subject. Such pharmaceutical compositions may be administered by any route of administration appropriate for the intended use, typically but not necessarily intravenously or subcutaneously, or at a particular site in the body.

In one embodiment, for any preparation used in the methods disclosed herein, the therapeutically effective amount or dose can be estimated initially from in vitro assays. For example, a dose can be formulated in animal models and such information can be used to determine useful doses more accurately in humans. In another embodiment, toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

The dose and dosing regimen are selected to provide an efficacious treatment for the subject or patient in need, and is tailored to the particular disease and/or other conditions of the subject. The dose level, dosing frequency (e.g., once, twice or three times a day, or less frequently such as twice a week, once a week, every 2, 3 or 4 weeks, for example) will be determined by the pharmacokinetics, severity of disease, potential side effects, tolerability, and resolution of the disease and/or symptoms of the subject. The duration of dosing, possible dosing holidays, and other aspects of the dosing regimen will be determined by the healthcare professional based on the foregoing and other relevant medical information.

Non-limiting examples of multivalent cytokines include those in the ensuing table.

| MULTIVALENT | |
|---|---|
| CYTOKINE | SEQUENCE |
| TETRAVALENT | MYRMQLLSCI ALSLALVTNS APTSSSTKKT QLQLEHLLLD |
| IL-2 | LQMILNGINN YKNPKLTRML TFKFYMPKKA TELKHLQCLE |
| | EELKPLEEVL NLAQSKNFHL RPRDLISNIN VIVLELKGSE |
| | TTFMCEYADE TATIVEFLNR WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT (SEQ ID NO: 2) |
| TRIVALENT IL-2 | MYRMQLLSCI ALSLALVTNS APTSSSTKKT QLQLEHLLLD |
| | LQMILNGINN YKNPKLTRML TFKFYMPKKA TELKHLQCLE |
| | EELKPLEEVL NLAQSKNFHL RPRDLISNIN VIVLELKGSE |
| | TTFMCEYADE TATIVEFLNR WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT (SEQ ID NO: 4) |
| PENTAVALENT | MYRMQLLSCI ALSLALVTNS APTSSSTKKT QLQLEHLLLD |
| IL-2 | LQMILNGINN YKNPKLTRML TFKFYMPKKA TELKHLQCLE |
| | EELKPLEEVL NLAQSKNFHL RPRDLISNIN VIVLELKGSE |
| | TTFMCEYADE TATIVEFLNR WITFCQSIIS TLT |
| | (SEQ ID NO: 5) |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT (SEQ ID NO: 5) |
| HEXAVALENT | MYRMQLLSCI ALSLALVTNS APTSSSTKKT QLQLEHLLLD |
| IL-2 | LQMILNGINN YKNPKLTRML TFKFYMPKKA TELKHLQCLE |
| | EELKPLEEVL NLAQSKNFHL RPRDLISNIN VIVLELKGSE |
| | TTFMCEYADE TATIVEFLNR WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT (SEQ ID NO: 6) |
| HEPTAVALENT | MYRMQLLSCI ALSLALVTNS APTSSSTKKT QLQLEHLLLD |
| IL-2 | LQMILNGINN YKNPKLTRML TFKFYMPKKA TELKHLQCLE |
| | EELKPLEEVL NLAQSKNFHL RPRDLISNIN VIVLELKGSE |
| | TTFMCEYADE TATIVEFLNR WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT (SEQ ID NO: 7) |
| OCTAVALENT | MYRMQLLSCI ALSLALVTNS APTSSSTKKT QLQLEHLLLD |
| IL-2 | LQMILNGINN YKNPKLTRML TFKFYMPKKA TELKHLQCLE |
| | EELKPLEEVL NLAQSKNFHL RPRDLISNIN VIVLELKGSE |
| | TTFMCEYADE TATIVEFLNR WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT |
| | GGGGS GGGGS GGGGS GGGGS |
| | APTSSSTKKT QLQLEHLLLD LQMILNGINN YKNPKLTRML |
| | TFKFYMPKKA TELKHLQCLE EELKPLEEVL NLAQSKNFHL |
| | RPRDLISNIN VIVLELKGSE TTFMCEYADE TATIVEFLNR |
| | WITFCQSIIS TLT (SEQ ID NO: 8) |
| BIVALENT IL-2 | MYRMQLLSCIALSLALVTNSAPTSSSTKKTQLQLEHLLLDLQMILNG |
| (forms tetravalent | INNYKNPKLTRMLTFKFYMPKKATELKHLQCLEEELKPLEEVLNLA |
| IL-2 by disulfide | QSKNFHLRPRDLISNINVIVLELKGSE |
| dimerization of Fc | TTFMCEYADETATIVEFLNRWITFCQSIISTLT |
| regions; SEQ ID | GGGGSGGGGSGGGGSGGGGS |
| NO: 23) | DKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQ |
| | DWLNGKEYKCKVSNKALPAPIEKTISKAKGQPREPQVYTLPPSRDE |
| | LTKNQVSLTCLVKGFYPSDIAVEWESNGQPENNYKTTPPVLDSDGS |
| | FFLYSKLTVDKSRWQQGNVFSCSVMHEALHNHYTQKSLSLSP |
| | GGGGGSGGGGSGGGGSGGGGS |
| | APTSSSTKKTQLQLEHLLLDLQMILNGINNYKNPKLTRMLTFKFYM |
| | PKKATELKHLQCLEEELKPLEEVLNLAQSKNFHLRPRDLISNINVIVL |
| | ELKGSETTFMCEYADETATIVEFLNRWITFCQSIISTLT (SEQ ID |
| | NO: 9). |
| TETRAVALENT | MYRMQLLSCIALSLALVTNSAPTSSSTKKTQLQLEHLLLDLQMILNG |
| IL-2 | INNYKNPKLTRMLTFKFYMPKKATELKHLQCLEEELKPLEEVLNLA |
| (forms octavalent | QSKNFHLRPRDLISNINVIVLELKGSE |
| IL-2 by disulfide | TTFMCEYADETATIVEFLNRWITFCQSIISTLT |
| dimerization of Fc | GGGGSGGGGSGGGGSGGGGS |
| regions; SEQ ID | APTSSSTKKTQLQLEHLLLDLQMILNGINNYKNPKLTRMLTFKFYM |
| NO: 24) | PKKATELKHLQCLEEELKPLEEVLNLAQSKNFHLRPRDLISNINVIVL |
| | ELKGSETTFMCEYADETATIVEFLNRWITFCQSIISTLT |
| | GGGGSGGGGSGGGGSGGGGS |
| | DKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQ |
| | DWLNGKEYKCKVSNKALPAPIEKTISKAKGQPREPQVYTLPPSRDE |
| | LTKNQVSLTCLVKGFYPSDIAVEWESNGQPENNYKTTPPVLDSDGS |
| | FFLYSKLTVDKSRWQQGNVFSCSVMHEALHNHYTQKSLSLSPG |
| | GGGGSGGGGSGGGGSGGGGS |
| | APTSSSTKKTQLQLEHLLLDLQMILNGINNYKNPKLTRMLTFKFYM |
| | PKKATELKHLQCLEEELKPLEEVLNLAQSKNFHLRPRDLISNINVIVL |
| | ELKGSETTFMCEYADETATIVEFLNRWITFCQSIISTLT |
| | GGGGSGGGGSGGGGSGGGGS |
| | APTSSSTKKTQLQLEHLLLDLQMILNGINNYKNPKLTRMLTFKFYM |
| | PKKATELKHLQCLEEELKPLEEVLNLAQSKNFHLRPRDLISNINVIVL |
| | ELKGSETTFMCEYADETATIVEFLNRWITFCQSIISTLT (SEQ ID |
| | NO: 10) |
| BIVALENT IL-7 | MFHVSFRYIFGLPPLILVLLPVASSDCDIEGKDGKQYESVLMVSIDQL |
| (forms tetravalent | LDSMKEIGSNCL |
| IL-7 by disulfide | NNEFNFFKRHICDANKEGMFLFRAARKLRQFLKMNSTGDFDLHLLK |
| dimerization of Fc | VSEGTTILLNCTGQVKGRKPAALGEAQPTKSLEENKSLKEQKKLND |
| regions; SEQ ID | LCFLKRLLQEIKTCWNKILMGTKEH |
| NO: 25) | GGGGSGGGGSGGGGSGGGGS |
| | DKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQ |
| | DWLNGKEYKCKVSNKALPAPIEKTISKAKGQPREPQVYTLPPSRDE |
| | LTKNQVSLTCLVKGFYPSDIAVEWESNGQPENNYKTTPPVLDSDGS |
| | FFLYSKLTVDKSRWQQGNVFSCSVMHEALHNHYTQKSLSLSPG |
| | GGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH (SEQ ID NO: 11). |
| TETRAVALENT | MFHVSFRYIFGLPPLILVLLPVASSDCDIEGKDG |
| IL-7 | KQYESVLMVSIDQLLDSMKEIGSNCLNNEFNFF |
| (forms octavalent | KRHICDANKEGMFLFRAARKLRQFLKMNSTGD |
| IL-7 by disulfide | FDLHLLKVSEGTTILLNCTGQVKGRKPAALGEA |
| dimerization of Fc | QPTKSLEENKSLKEQKKLNDLCFLKRLLQEIKTC |
| regions; SEQ ID | WNKILMGTKEH |
| NO: 75) | GGGGGSGGGGSGGGGSGGGGS |
| | DCDIEGKDGKQYESVLMVSIDQLLDSMKEIGSNCLNNEFNFF |
| | KRHICDANKEGMFLFRAARKLRQFLKMNSTGD |
| | FDLHLLKVSEGTTILLNCTGQVKGRKPAALGEA |
| | QPTKSLEENKSLKEQKKLNDLCFLKRLLQEIKTC |
| | WNKILMGTKEH |
| | GGGGGSGGGGSGGGGSGGGGS |
| | DKTHTCPPCPAPELLGGPSVFLFPPKPKDTL |
| | MISRTPEVTCVVVDVSHEDPEVKFNWYVD |
| | GVEVHNAKTKPREEQYNSTYRVVSVLTVL |
| | HQDWLNGKEYKCKVSNKALPAPIEKTISKA |
| | KGQPREPQVYTLPPSRDELTKNQVSLTCLVKG |
| | FYPSDIAVEWESNGQPENNYKTTPPVLDSDG |
| | SFFLYSKLTVDKSRWQQGNVFSCSVMHEALHN |
| | HYTQKSLSLSP |
| | GGGGGSGGGGSGGGGSGGGGS |
| | DCDIEGKDGKQYESVLMVSIDQLLDSMKEIGSNCLNNEFNFF |
| | KRHICDANKEGMFLFRAARKLRQFLKMNSTGD |
| | FDLHLLKVSEGTTILLNCTGQVKGRKPAALGEA |
| | QPTKSLEENKSLKEQKKLNDLCFLKRLLQEIKTC |
| | WNKILMGTKEH |
| | GGGGGSGGGGSGGGGSGGGGS |
| | DCDIEGKDGKQYESVLMVSIDQLLDSMKEIGSNCLNNEFNFF |
| | KRHICDANKEGMFLFRAARKLRQFLKMNSTGD |
| | FDLHLLKVSEGTTILLNCTGQVKGRKPAALGEA |
| | QPTKSLEENKSLKEQKKLNDLCFLKRLLQEIKTC |
| | WNKILMGTKEH (SEQ ID NO: 74) |
| TRIVALENT IL-7 | MFHVSFRYIFGLPPLILVLLPVASSDCDIEGKDGKQYESVLMVSIDQL |
| TETRAVALENT | LDSMKEIGSNCLNNEFNFFKRHICDANKEGMFLFRAARKLRQFLKM |
| IL-7 | NSTGDFDLHLLKVSEGTTILLNCTGQVKGRKPAALGEAQPTKSLEE |
| | NKSLKEQKKLNDLCFLKRLLQEIKTCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH (SEQ ID NO: 22) |
| | MFHVSFRYIFGLPPLILVLLPVASSDCDIEGKDGKQYESVLMVSIDQL |
| | LDSMKEIGSNCLNNEFNFFKRHICDANKEGMFLFRAARKLRQFLKM |
| | NSTGDFDLHLLKVSEGTTILLNCTGQVKGRKPAALGEAQPTKSLEE |
| | NKSLKEQKKLNDLCFLKRLLQEIKTCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH (SEQ ID NO: 17) |
| PENTAVALENT | MFHVSFRYIFGLPPLILVLLPVASSDCDIEGKDGKQYESVLMVSIDQL |
| IL-7 | LDSMKEIGSNCLNNEFNFFKRHICDANKEGMFLFRAARKLRQFLKM |
| | NSTGDFDLHLLKVSEGTTILLNCTGQVKGRKPAALGEAQPTKSLEE |
| | NKSLKEQKKLNDLCFLKRLLQEIKTCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH (SEQ ID NO: 18) |
| HEXAVALENT | MFHVSFRYIFGLPPLILVLLPVASSDCDIEGKDGKQYESVLMVSIDQL |
| IL-7 | LDSMKEIGSNCLNNEFNFFKRHICDANKEGMFLFRAARKLRQFLKM |
| | NSTGDFDLHLLKVSEGTTILLNCTGQVKGRKPAALGEAQPTKSLEE |
| | NKSLKEQKKLNDLCFLKRLLQEIKTCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH (SEQ ID NO: 19) |
| HEPTAVALENT | MFHVSFRYIFGLPPLILVLLPVASSDCDIEGKDGKQYESVLMVSIDQL |
| IL-7 | LDSMKEIGSNCLNNEFNFFKRHICDANKEGMFLFRAARKLRQFLKM |
| | NSTGDFDLHLLKVSEGTTILLNCTGQVKGRKPAALGEAQPTKSLEE |
| | NKSLKEQKKLNDLCFLKRLLQEIKTCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH (SEQ ID NO: 20) |
| OCTAVALENT | MFHVSFRYIFGLPPLILVLLPVASSDCDIEGKDGKQYESVLMVSIDQL |
| IL-7 | LDSMKEIGSNCLNNEFNFFKRHICDANKEGMFLFRAARKLRQFLKM |
| | NSTGDFDLHLLKVSEGTTILLNCTGQVKGRKPAALGEAQPTKSLEE |
| | NKSLKEQKKLNDLCFLKRLLQEIKTCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH |
| | GGGGGSGGGGSGGGGSGGGGS |
| | DCDIEGKDGKQYESVLMVSIDQLLDSMKEIGSNCLNNEFNFFKRHIC |
| | DANKEGMFLFRAARKLRQFLKMNSTGDFDLHLLKVSEGTTILLNCT |
| | GQVKGRKPAALGEAQPTKSLEENKSLKEQKKLNDLCFLKRLLQEIK |
| | TCWNKILMGTKEH (SEQ ID NO: 21) |

In other embodiments, multivalent cytokines are prepared by further dimerization or multimerization of any of the foregoing sequences. For example, in one embodiment, hexavalent IL-2 is prepared by disulfide linking of Fc portions of SEQ ID NO:9 and SEQ ID NO:10. In some embodiments, the desired multivalent cytokine is isolated or purified from a reaction mixture used for the manufacture of the multivalent cytokines described herein.

In any embodiment herein, the common γ-chain (γ_{c}) receptor cytokines, such as interleukin (IL)-2, 4, 7, 9, 15, and 21, or an immunologically active fragment thereof, may comprise one or more modifications, such as but not limited to an amino acid modification such as an amino acid substitution, insertion, and/or deletion; truncation; modification of a (free) N- or C-terminus; and/or a post-translational modification such as but not limited to glycosylation, acylation, phosphorylation, deamidation, pegylation or sulphation. A non-limiting example of such muteins are IL-2 muteins with R38Q and/or H16N mutations; numerous other muteins of the common 7-chain receptor cytokines comprising the multivalent cytokines disclosed herein are known in the art and are embraced herein. Another example is IL-2 superkine (SEQ ID NO:76) that may be used in the IL-2-comprising multivalent cytokines disclosed herein. Silva et al., 2019, De novo design of potent and selective mimics of IL-2 and IL-15, Nature 565:186-191, describe other modified forms of IL-2 as well as IL-15, such as neoleukin-2/15 (Neo-2/15), that may be used in the multivalent cytokines disclosed herein. Such modifications in one embodiment enhance the biological activity, receptor binding activity, receptor affinity, receptor avidity, half-life, resistance to degradation, resistance to metabolism, resistance to proteolysis, and/or other features that modify and/or improve one or more features of the multivalent cytokines disclosed herein for clinical use, dosing, effective and/or convenient dosing regimen, administration, storage, stability, ease of manufacturing, or other factors, in any combination. Any such modification may also be provided on fragments of the common γ-chain receptor cytokines disclosed herein, which retain their activity for the purposes described herein and hence referred to immunologically active fragments.

In another one embodiment, the activity of a multivalent cytokine disclosed herein on elevating T_{reg}s in a patient undergoing treatment may be assessed by determining the T_{reg} abundance in a blood sample from the patient, which may be determined over time, e.g., during and after the treatment period. In some embodiments, titration of the dose level in a patient is carried out by measuring T_{reg} levels periodically and adjusting the dose or dose regimen. In some embodiments, determining the optimal effective dose or dose regimen of a multivalent cytokine in a clinical study, may be carried out by conducting a dose response study to identify the highest dose of multivalent cytokine that expands the T_{reg} population without expanding other T cell populations such as helper T cells and/or NK cells. Such monitoring of activity may be provided during clinical development of a multivalent cytokine, or recommended monitoring for patients receiving treatment.

As used herein, the terms “comprise”, “comprises”, “comprising”, “includes”, “including”, “having” and their conjugates mean “including but not limited to”.

As used herein, the singular form “a”, “an” and “the” include plural references unless the context clearly dictates otherwise. For example, the term “an enzyme” or “at least one enzyme” may include a plurality of enzymes, including mixtures thereof.

Throughout this application, various embodiments of the present disclosure may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of the invention and variations thereof are described. This description is not intended to be limiting and changes in the components, sequence of steps, and other variations would be understood to be within the scope of the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Example 1

### Multivalency Enhances the Specificity of Fc-Cytokine Fusions

### Materials and Methods

### Binding Model

Model was formulated as described in Tan and Meyer, A general model of multivalent binding with ligands of heterotypic subunits and multiple surface receptors. Mathematical Biosciences 2021 December; 342: 108714. The monomer composition of a ligand complex can be represented by a vector θ=(θ₁, θ₂ . . . , θ_{N}_{_{L}}), where each θᵢ is the number of monomer ligand type i on that complex. Let C_{θ} be the proportion of the θ complexes in all ligand complexes, and Θ be the set of all possible θ's, Σ_{θ∈Θ}C_{θ}=1.

The binding between a ligand complex and a cell expressing several types of receptors can be represented by a series of qᵢⱼ. The relationship between qᵢⱼ's and θᵢ is given by θᵢ=qᵢ₀+qᵢ₁+ . . . +q_{iN}_{_{R}}. Let the vector qᵢ=(qᵢ₀, qᵢ₁, . . . , q_{iN}_{_{R}}), and the corresponding θ of a binding configuration q be θ(q). For all i in {1, 2, . . . , N_{L}}, ψᵢⱼ=R_{eq,j}K_{a,ij}K*ₓ, where j={1, 2, . . . , N_{R}} and ψᵢ₀=1. The relative number of complexes bound to a cell with configuration q at equilibrium is

$${v}_{q , eq} = \frac{{L}_{0} {C}_{θ ( q )}}{{K}_{x}^{*}} {\prod }_{\begin{matrix}i = 1 \\ j = 0\end{matrix}}^{\begin{matrix}i = {N}_{L} \\ j = {N}_{R}\end{matrix}} {ψ}_{ij}^{{q}_{ij}} {\prod }_{i = 1}^{{N}_{L}} ( \begin{matrix}{θ}_{i} \\ {q}_{i}\end{matrix} ) .$$

Then the relative amount of bound receptor n can be calculated as

$${R}_{bound , n} = \frac{{L}_{0}}{{K}_{x}^{*}} {\sum }_{θ ∈ Θ} {C}_{θ} [ {\sum }_{i = 1}^{{N}_{L}} \frac{{ψ}_{in} {θ}_{i}}{{\sum }_{j = 0}^{{N}_{R}} {ψ}_{ij}} ] {\prod }_{i = 1}^{{N}_{L}} {( {\sum }_{j = 0}^{{N}_{R}} {ψ}_{ij} )}^{{θ}_{i}} .$$

By R_{tot,n}=R_{eq,n}+R_{bound,n}, one can solve R_{eq,n} numerically for each type of receptor.

### Application of Multivalent Binding Model to IL-2 Signaling Pathway

Each IL-2 molecule was allowed to bind to one free IL-2Rα and one IL-2Rβ/γ_{c} receptor. Initial IL-2 receptor association proceeds with the known kinetics of monomeric ligand-receptor interaction (Table 1). Subsequent ligand-receptor binding interactions then proceed with an associatioronstant proportional to available receptor abundance and affinity multiplied by the scaling constant, Kₓ*, as described above. To predict pSTAT5 response to IL-2 stimulation, it is assumed that pSTAT5 is proportional to the amount of IL-2-bound IL-2Rβ/γ_{c}, as complexes which contain these species actively signal through the JAK/STAT pathway. Scaling factors converting from predicted active signaling species to pSTAT5 abundance were fit to experimental data on a per-experiment and cell type basis. A single Kₓ* value was fit to all experiments and cell types.

TABLE 1
IL-2 Variants' Affinities for IL-2R Subunits

| | IL-2Rα | IL-2Rβ/γ_{c} |
|---|---|---|
| Ligand | Affinity (K_{D}, nM) | Affinity (K_{D}, nM) |
| WT IL-2 | 10.0 | 0.133 |
| WT N-term | 0.19 | 5.296 |
| WT C-term | 0.54 | 3.043 |
| V91K C-term | 0.69 | 7.5586 |
| R38Q N-term | 0.71 | 3.9949 |
| F42Q N-Term | 9.48 | 2.815 |
| N88D C-term | 1.01 | 24.0166 |
| H16N N-term | 0.43 | 22.35 |
| R38Q/H16N | 0.71 | 22.35 |

### Tensor Factorization

Before decomposition, the signaling response data was background subtracted and variance scaled across each cell population. Tensor decomposition was performed using the Python package TensorLy, using non-negative canonical polyadic decomposition.

### Receptor Abundance Quantitation

Cryopreserved PBMCs (ATCC, PCS-800-011, lot #81115172) were thawed to room temperature and slowly diluted with 9 mL pre-warmed RPMI-1640 medium (Gibco, 11875-093) supplemented with 10% fetal bovine serum (FBS, Seradigm, 1500-500, lot #322B15). Media was removed, and cells washed once more with 10 mL warm RPMI-1640+10% FBS. Cells were brought to 1.5×10⁶ cells/mL, distributed at 250,000 cells per well in a 96-well V-bottom plate, and allowed to recover 2 hrs at 37° C. in an incubator at 5% CO₂. Cells were then washed twice with PBS+0.1% BSA (PBSA, Gibco, 15260-037, Lot #2000843) and suspended in 50 μL PBSA+10% FBS for 10 min on ice to reduce background binding to IgG.

Antibodies were diluted in PBSA+10% FBS and cells were stained for 1 hr at 4° C. in darkness with a gating panel (Panel 1, Panel 2, Panel 3, or Panel 4) and one anti-receptor antibody, or an equal concentration of matched isotype/fluorochrome control antibody. Stain for CD25 was included in Panel 1 when CD122, CD132, CD127, or CD215 was being measured (CD25 is used to separate T_{reg}s from other CD4⁺ T cells).

Compensation beads (Simply Cellular Compensation Standard, Bangs Labs, 550, lot #12970) and quantitation standards (Quantum Simply Cellular anti-Mouse IgG or anti-Rat IgG, Bangs Labs, 815, Lot #13895, 817, Lot #13294) were prepared for compensation and standard curve. One well was prepared for each fluorophore with 2 μL antibody in 50 μL PBSA and the corresponding beads. Bead standards were incubated for 1 hr at room temperature in the dark.

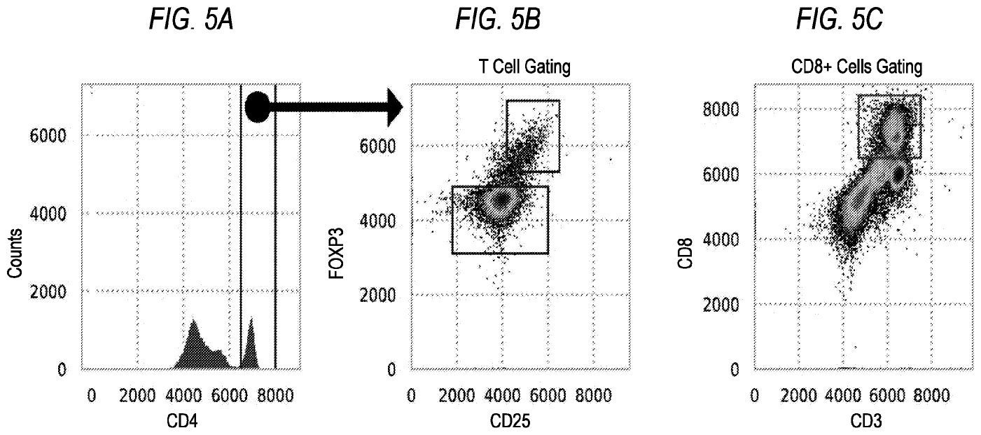

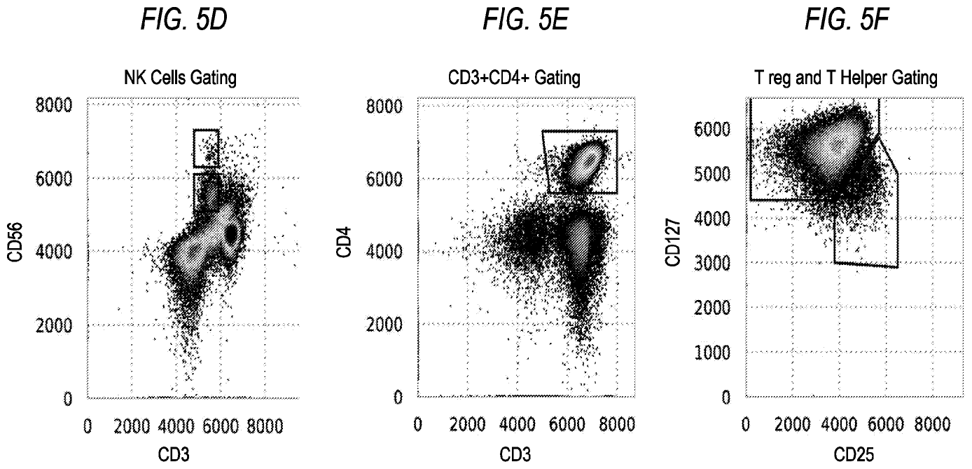

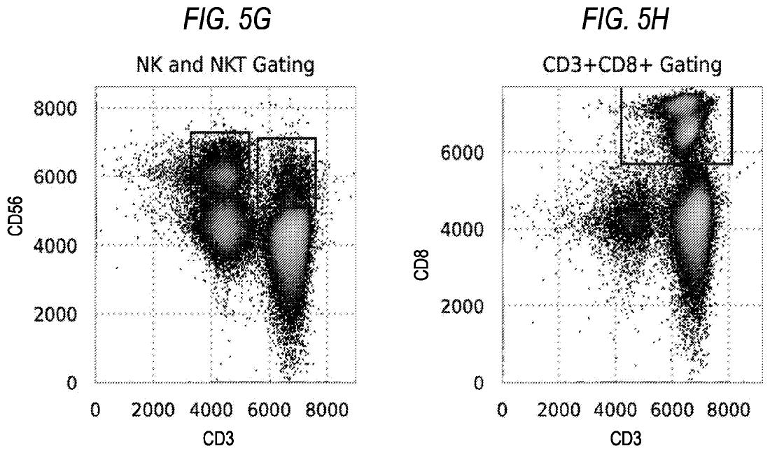

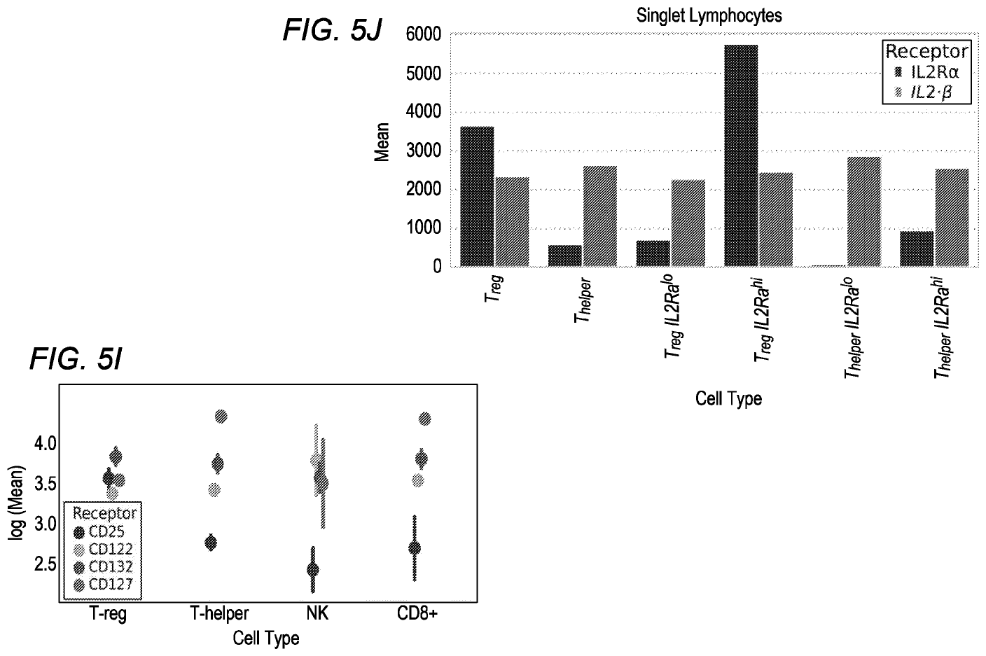

Both beads and cells were washed twice with PBSA. Cells were suspended in 120 μL per well PBSA, and beads to 50 μL, and analyzed using an IntelliCyt iQue Screener PLUS with VBR configuration (Sartorius) with a sip time of 35 and 30 secs for cells and beads, respectively. Antibody number was calculated from fluorescence intensity by subtracting isotype control values from matched receptor stains and calibrated using the two lowest binding quantitation standards. T_{reg} cells could not be gated in the absence of CD25, so CD4⁺ T cells were used as the isotype control to measure CD25 in T_{reg} populations. Cells were gated as shown in FIG. 5. Measurements were performed using four independent staining procedures over two days. Separately, the analysis was performed with anti-receptor antibodies at 3× normal concentration to verify that receptor binding was saturated. Replicates were summarized by geometric mean.

pSTAT5 Measurement of IL-2 and -15 Signaling in PBMCs

Human PBMCs were thawed, distributed across a 96-well plate, and allowed to recover as described above. IL-2 (R&D Systems, 202-IL-010) or IL-15 (R&D Systems, 247-ILB-025) were diluted in RPMI-1640 without FBS and added to the indicated concentrations. To measure pSTAT5, media was removed, and cells fixed in 100 μL of 10% formalin (Fisher Scientific, SF100-4) for 15 mins at room temperature.

Formalin was removed, cells were placed on ice, and cells were gently suspended in 50 μL of cold methanol (−30° C.). Cells were stored overnight at −30° C. Cells were then washed twice with PBSA, split into two identical plates, and stained 1 hr at room temperature in darkness using antibody panels 4 and 5 with 50 μL per well. Cells were suspended in 100 μL PBSA per well, and beads to 50 μL, and analyzed on an IntelliCyt iQue Screener PLUS with VBR configuration (Sartorius) using a sip time of 35 seconds and beads 30 seconds. Compensation was performed as above. Populations were gated as shown in FIG. 5, and the median pSTAT5 level was extracted for each population in each well.

### Recombinant Proteins

IL-2/Fc fusion proteins were expressed using the Expi293 expression system according to manufacturer instructions (Thermo Scientific). Proteins were as human IgG1 Fc fused at the N- or C-terminus to human IL-2 through a (G4S)4 linker. C-terminal fusions omitted the C-terminal lysine residue of human IgG1. The AviTag sequence GLNDIFEAQKIEWHE (SEQ ID NO:3) was included on whichever terminus did not contain IL-2. Fc mutations to prevent dimerization were introduced into the Fc sequence. Proteins were purified using MabSelect resin (GE Healthcare). Proteins were biotinylated using BirA enzyme (BPS Biosciences) according to manufacturer instructions, and extensively buffer-exchanged into phosphate buffered saline (PBS) using Amicon 10 kDa spin concentrators (EMD Millipore). The sequence of IL-2Rβ/γ Fc heterodimer was based on a reported active heterodimeric molecule (patent application US20150218260A1), with the addition of (G4S)2 linker between the Fc and each receptor ectodomain. The protein was expressed in the Expi293 system and purified on MabSelect resin as above. IL2-Ra ectodomain was produced with C-terminal 6×His tag and purified on Nickel-NTA spin columns (Qiagen) according to manufacturer instructions.

### Octet Binding Assays

Binding affinity was measured on an Octet RED384 (ForteBio). Briefly, biotinylated monomericIL-2/Fc fusion proteins were uniformly loaded to Streptavidin biosensors (ForteBio) at roughly 10% of saturation point and equilibrated for 10 minutes in PBS+0.1% bovine serum albumin (BSA). Association time was up to 40 minutes in IL-2Rβ/γ titrated in 2× steps from 400 nM to 6.25 nM, or IL-2Rα from 25 nM to 20 pM, followed by dissociation in PBS+0.1% BSA. A zero-concentration control sensor was included in each measurement and used as a reference signal. Assays were performed in quadruplicate across two days. Binding to IL-2Rα did not fit to a simple binding model so equilibrium binding was used to determine the K_{D} within each assay. Binding to IL-2Rβ/γ fit a 1:1 binding model so on-rate (kₒₙ), off-rate (k_{off}) and K_{D} were determined by fitting to the entire binding curve. Kinetic parameters and K_{D} were calculated for each assay by averaging all concentrations with detectable binding signal (typically 12.5 nM and above).

### Results

### Systematic Signaling Profiling of IL-2 Variants Reveals Multitude of Determinants of Response

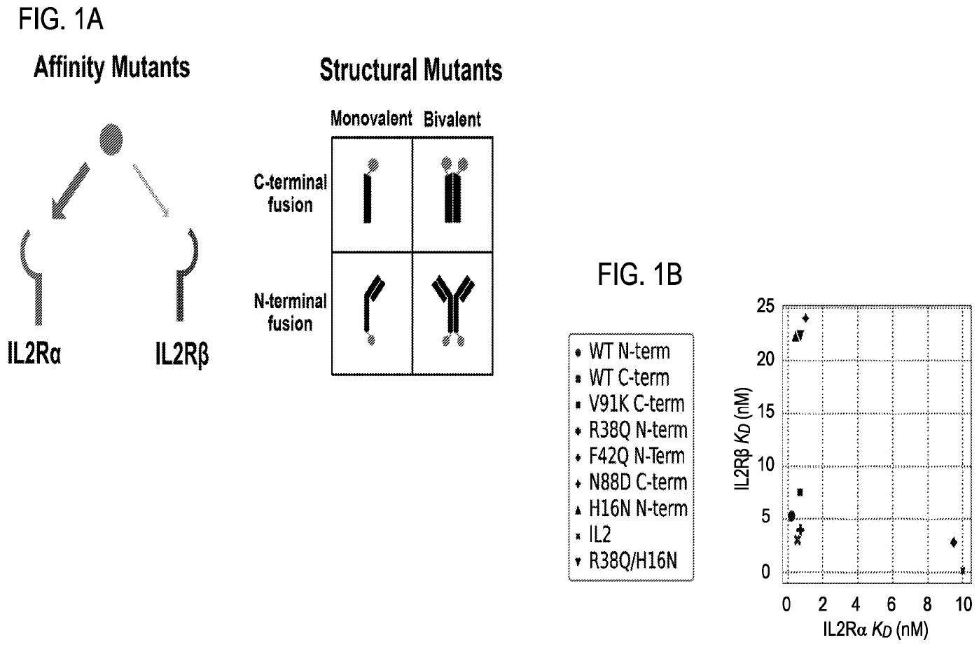

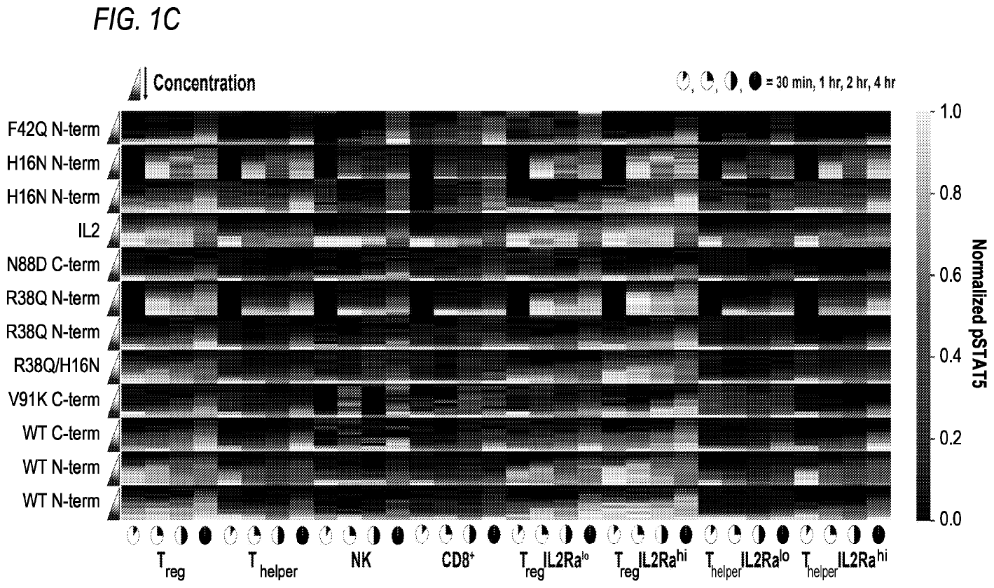

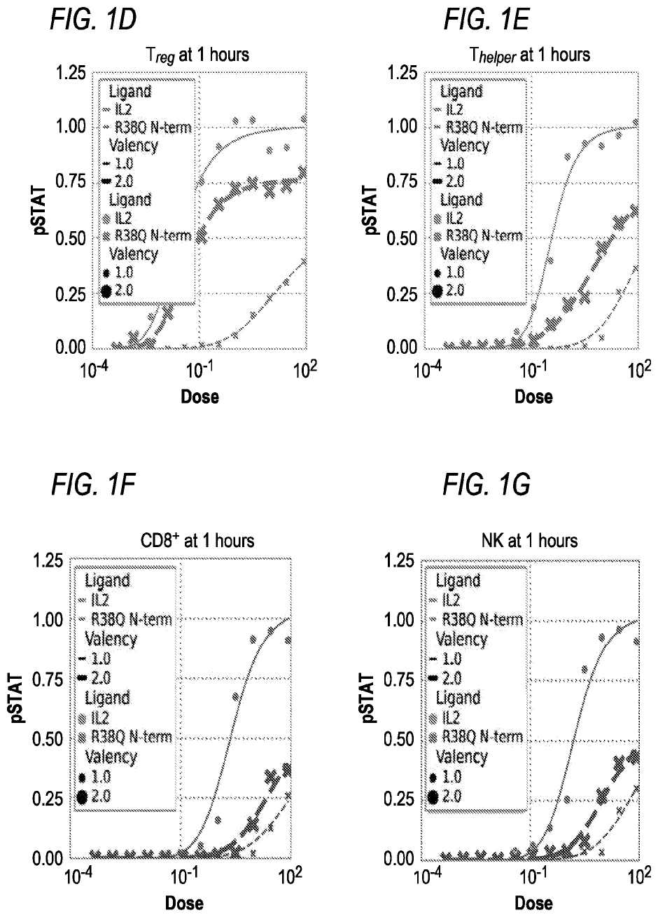

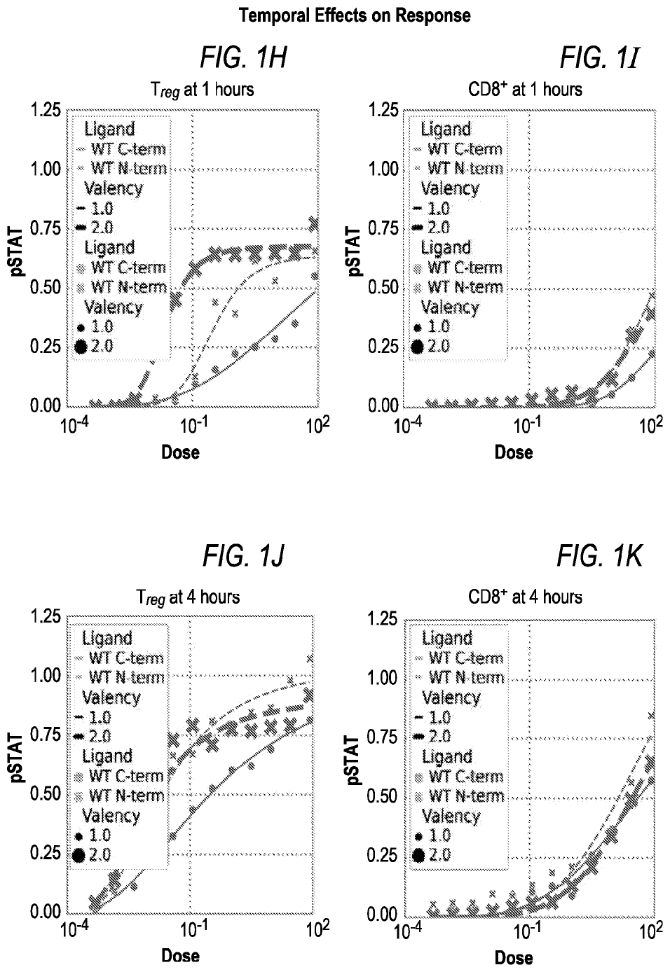

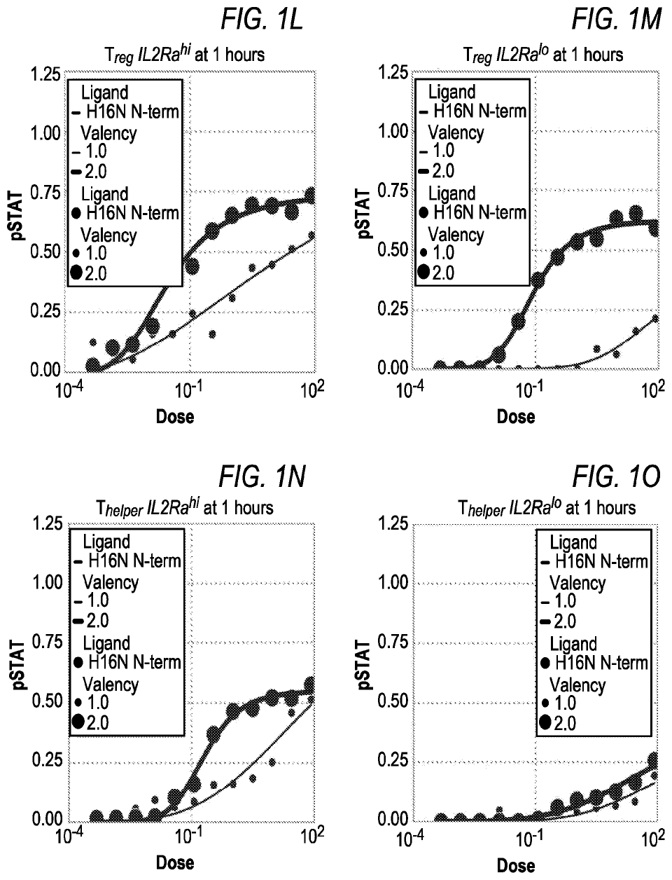

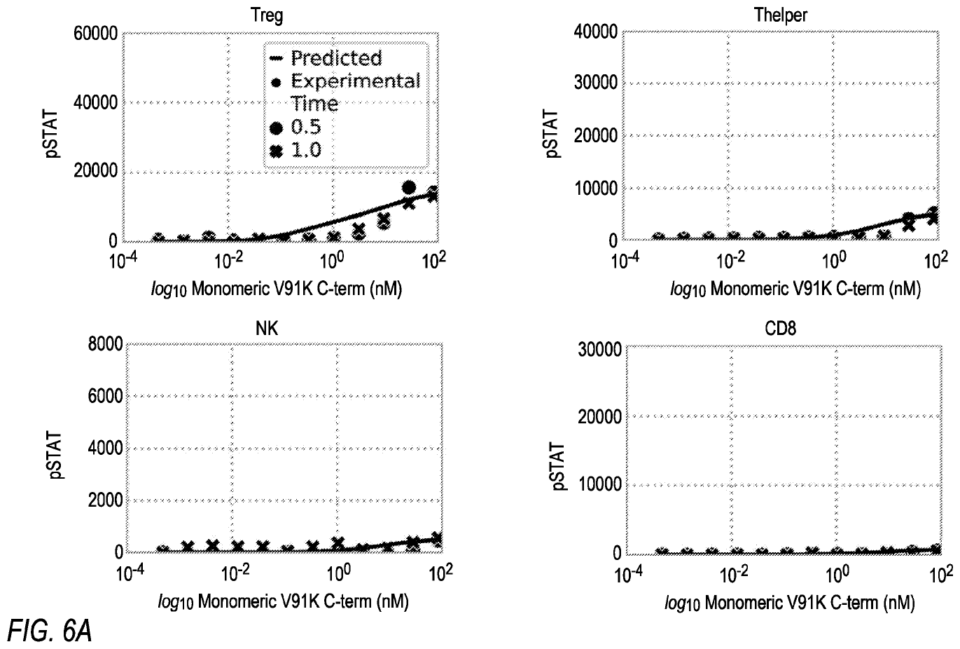

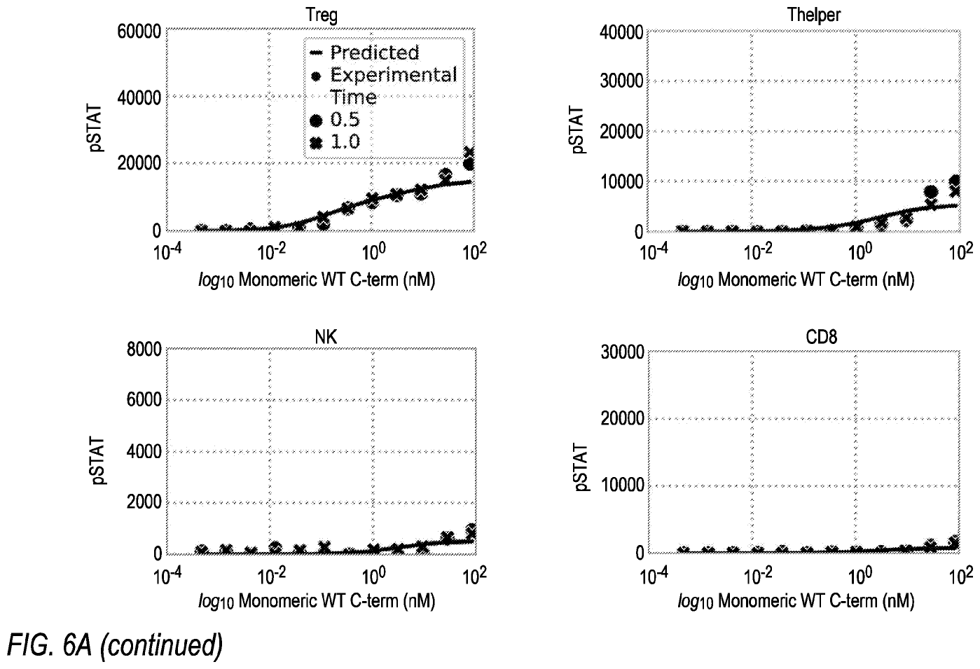

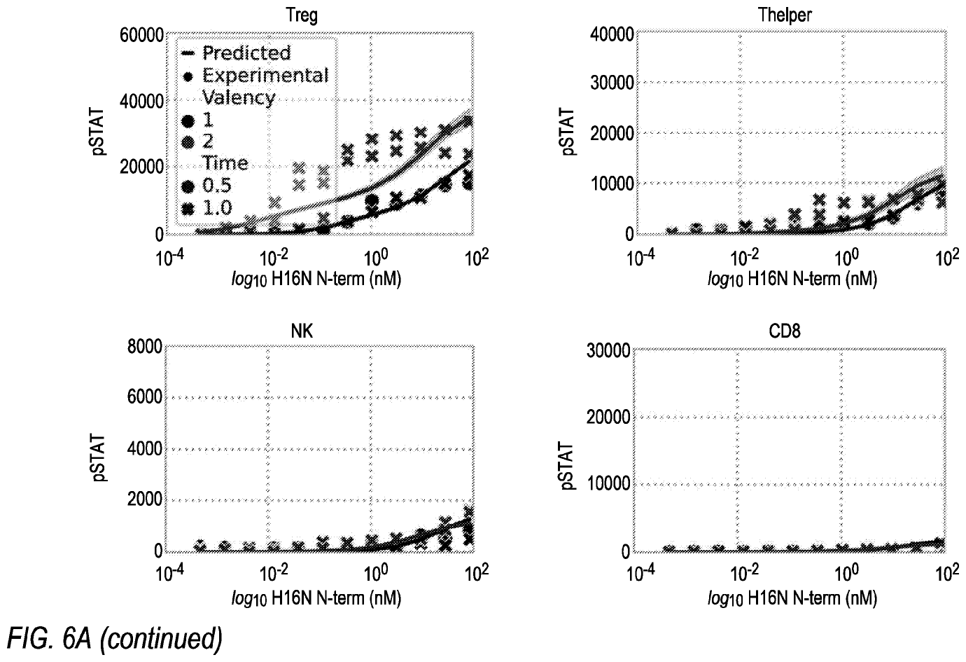

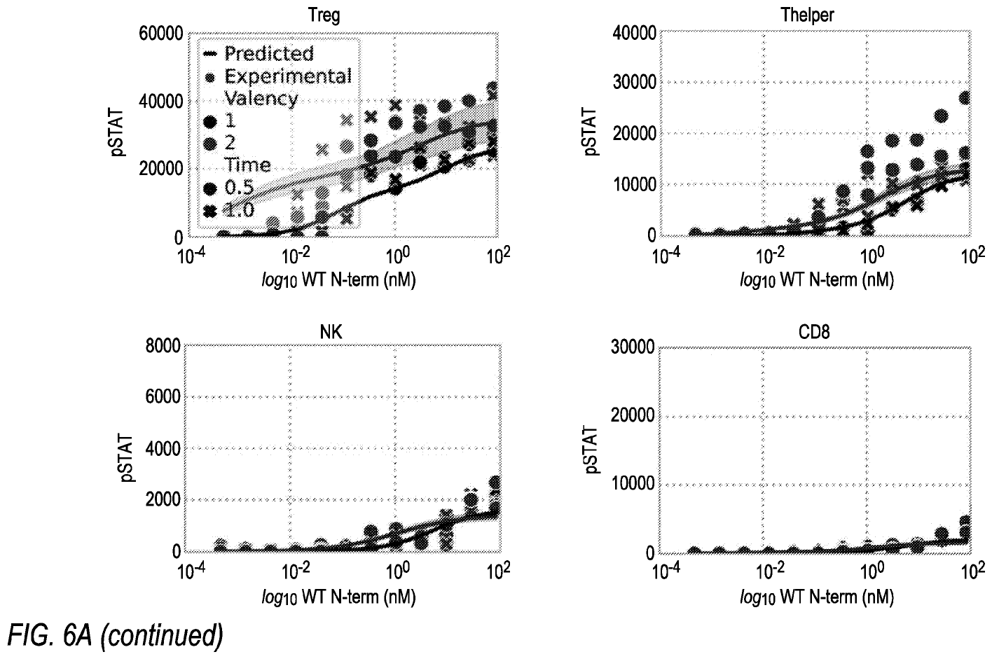

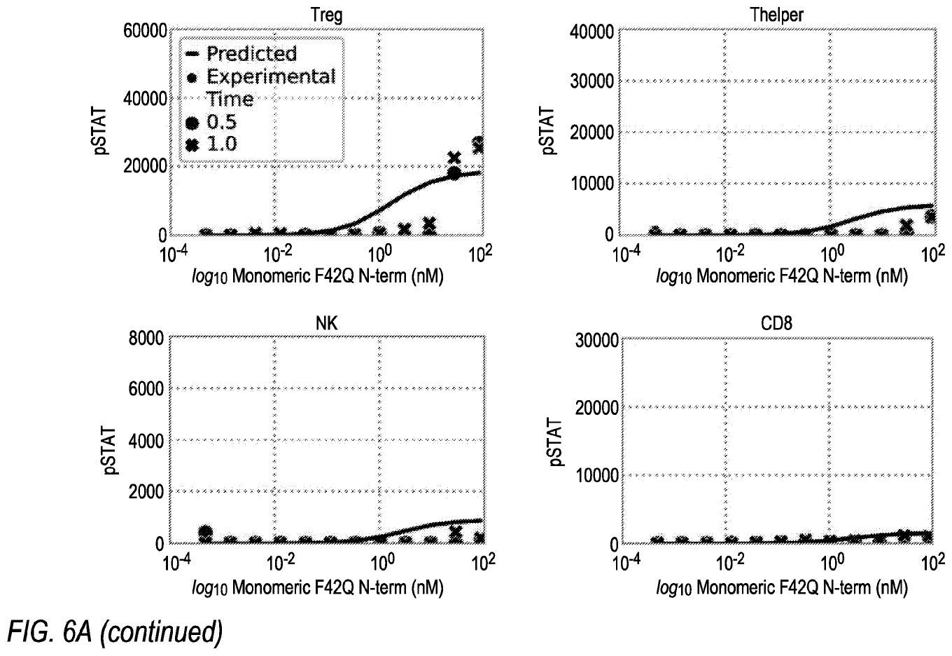

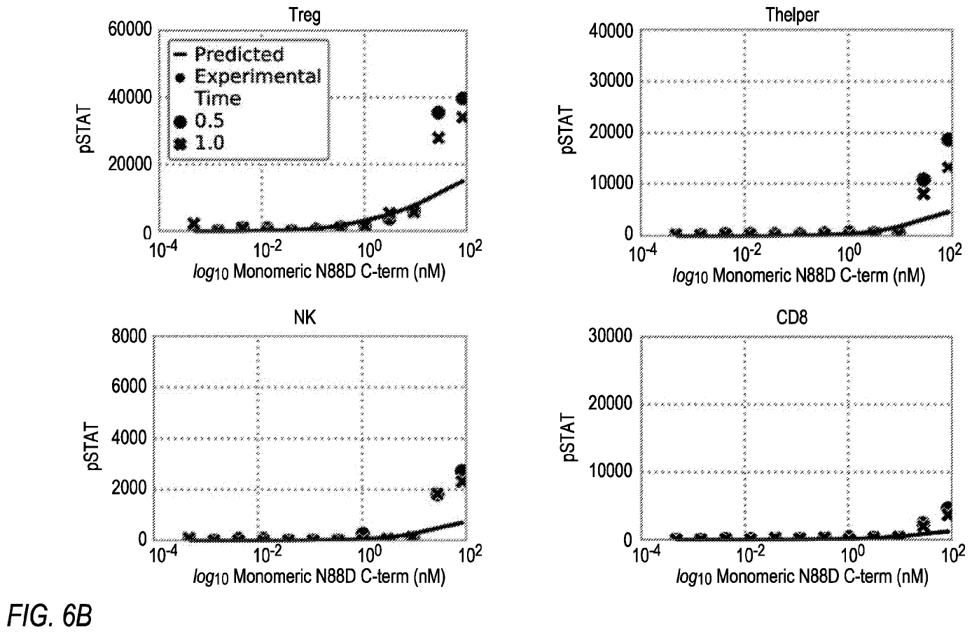

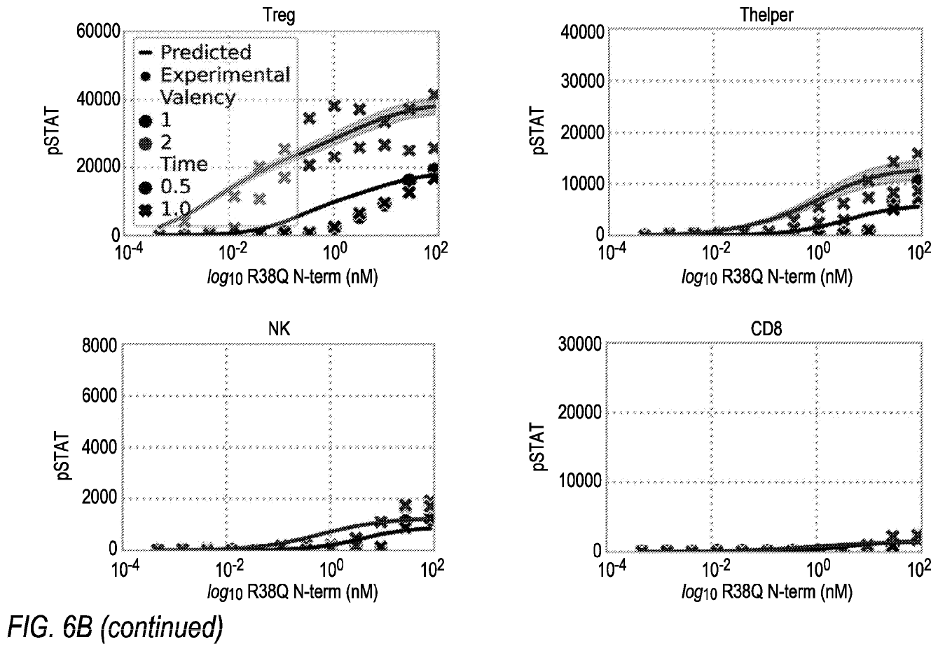

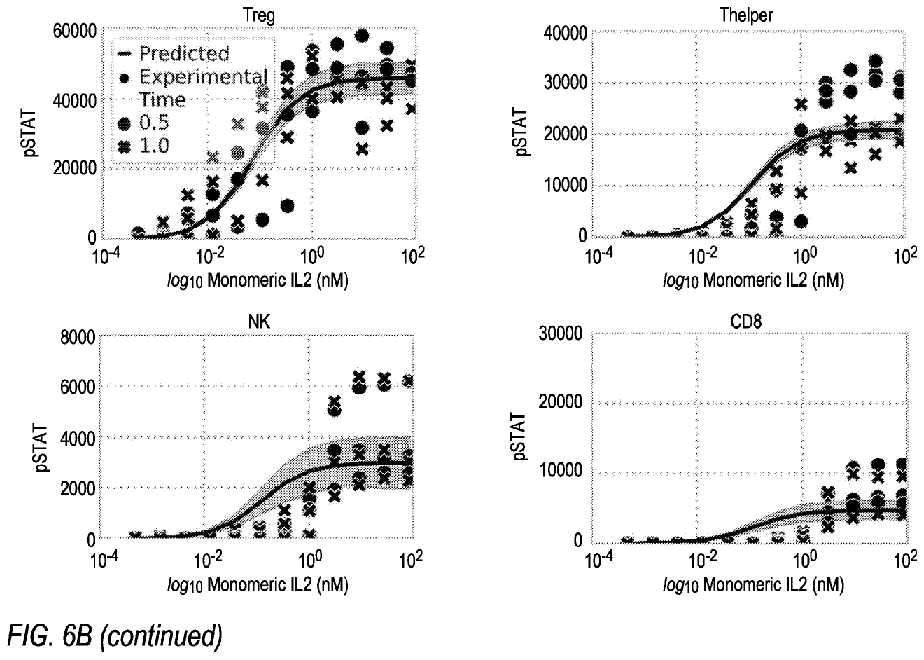

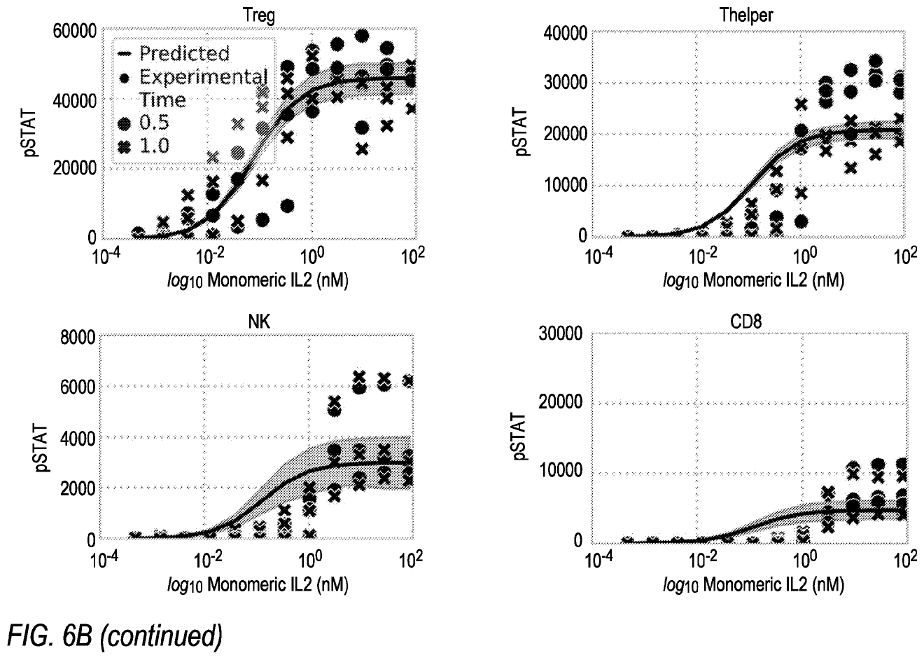

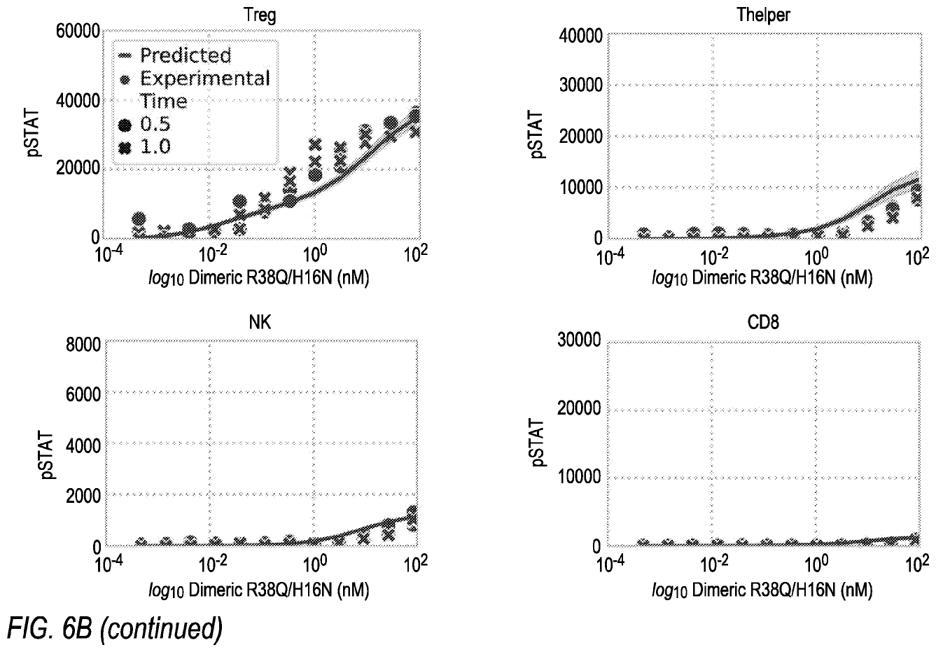

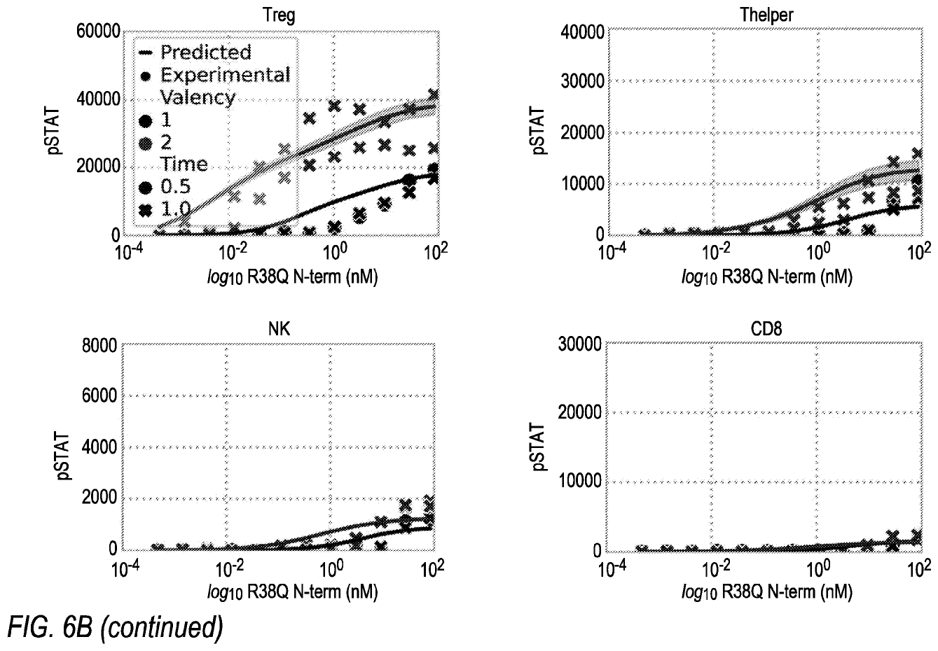

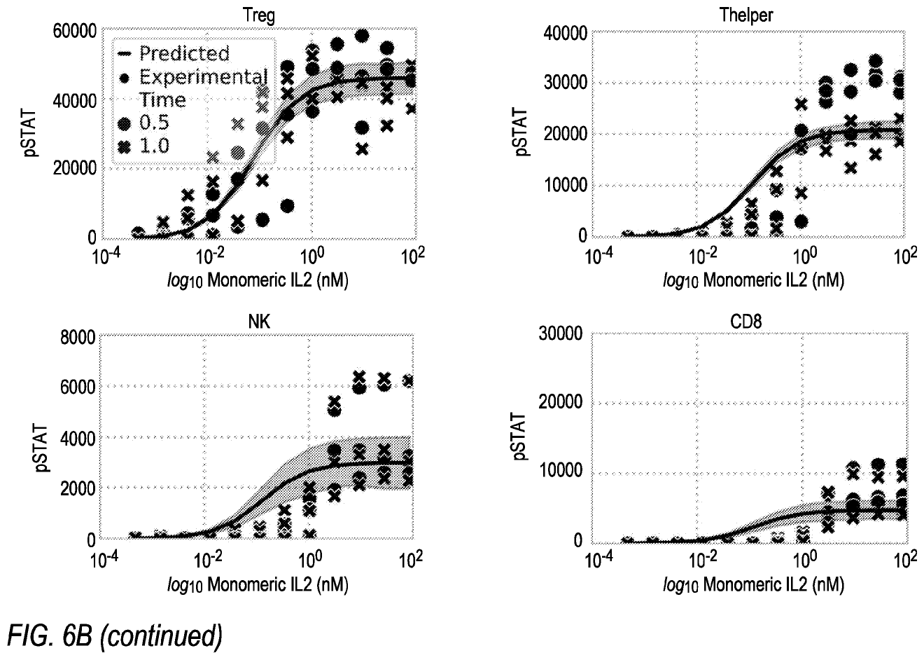

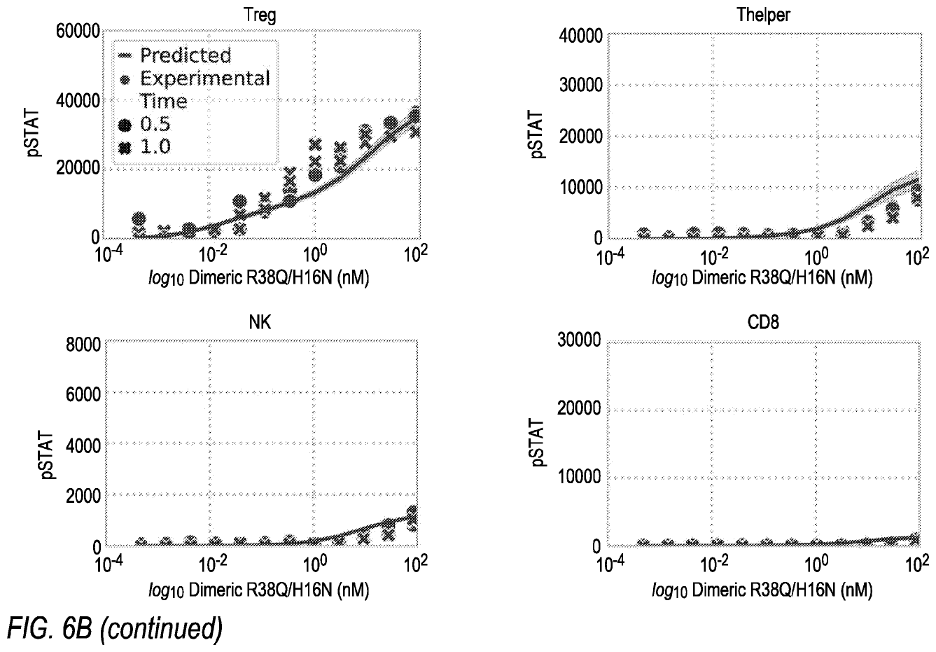

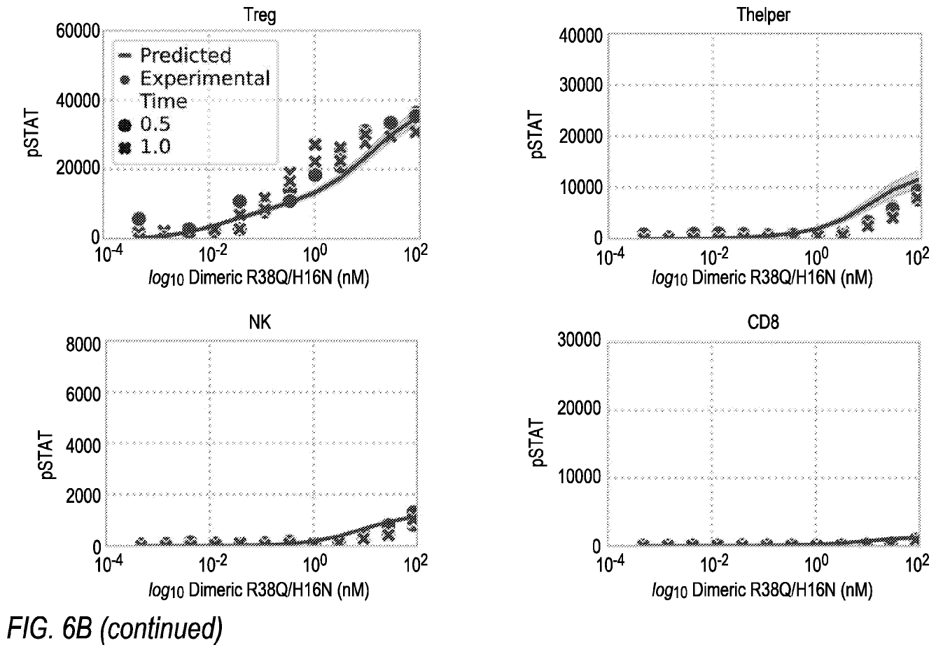

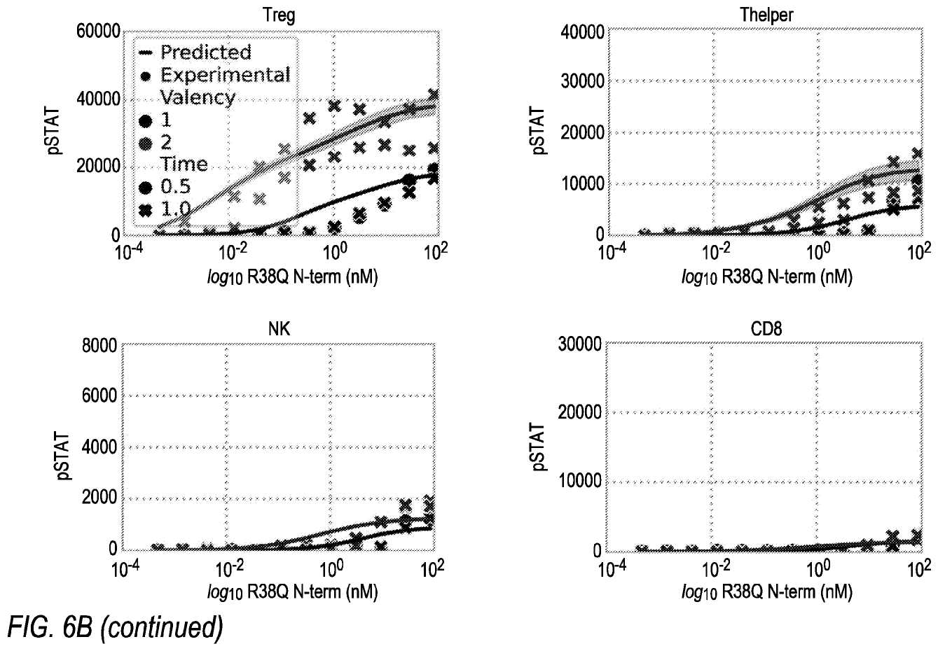

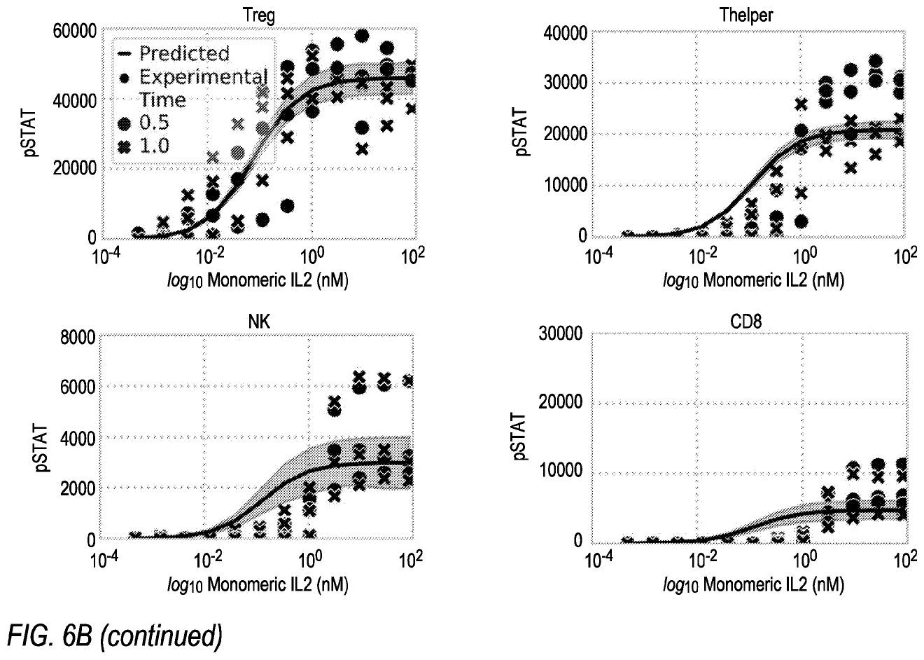

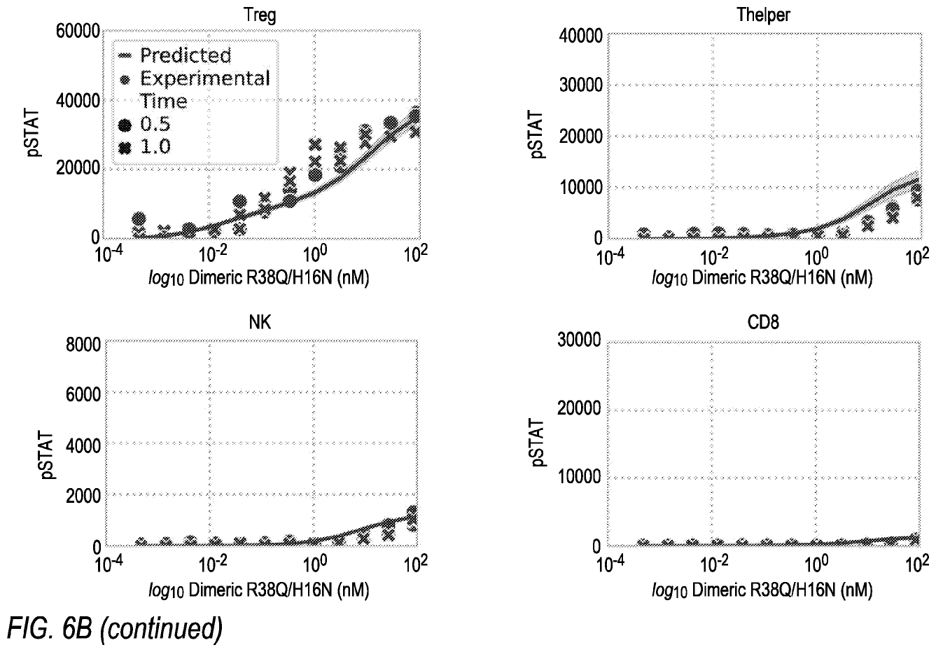

To explore the determinants of IL-2 response in a systematic manner, we stimulated peripheral blood mononuclear cells (PBMCs) collected from a single donor with 13 IL-2 muteins with varying affinities for IL2Rα and IL2Rβ in both monomeric and dimeric Fc-fusion formats (FIG. 1A-1B, Table 1). Here, affinity-engineered mutants were conjugated to IgG1 Fc fragments at either their N- or C-terminus. Stimulated cells were collected at four time points using 12 treatment concentrations. The PBMCs were then stained for canonical cell type markers and pSTAT5, a canonical read-out of signaling response, allowing us to separate signaling response by cell type. Four different cell types (T_{reg}, Tₕₑₗₚₑᵣ, CD8+, NK) were gated and quantified (FIG. 6A-B). T_{reg} and Tₕₑₗₚₑᵣ cells were further dissected according to their IL2Rα abundances into low, average, and high expression subpopulations by isolating sub-populations using three evenly logarithmically spaced bins (FIG. 6A-B). To get a surface level visualization of the effects of time, cell type, receptor abundance, ligand format and affinity, and concentration, we organized our signaling data into a heatmap (FIG. 1C).

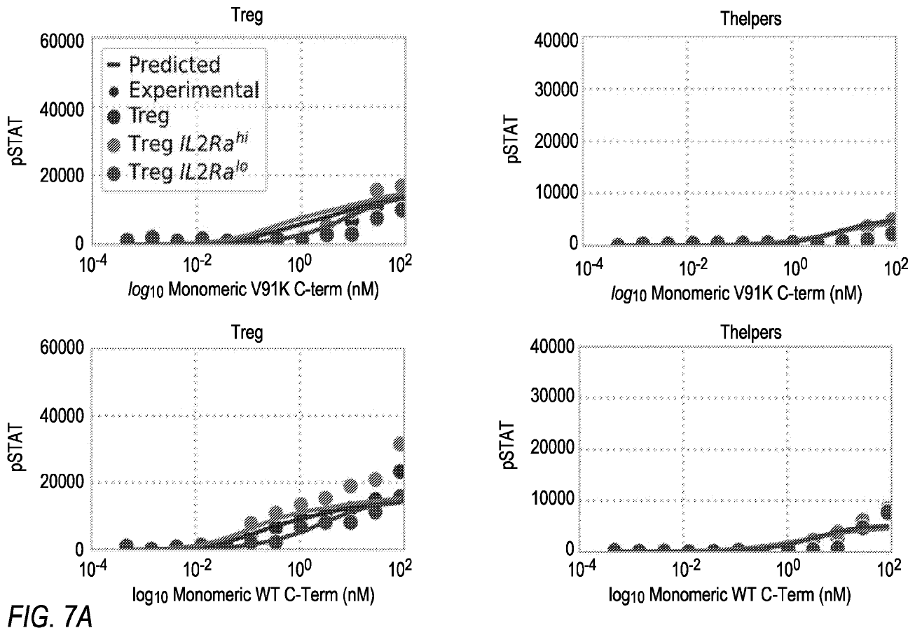

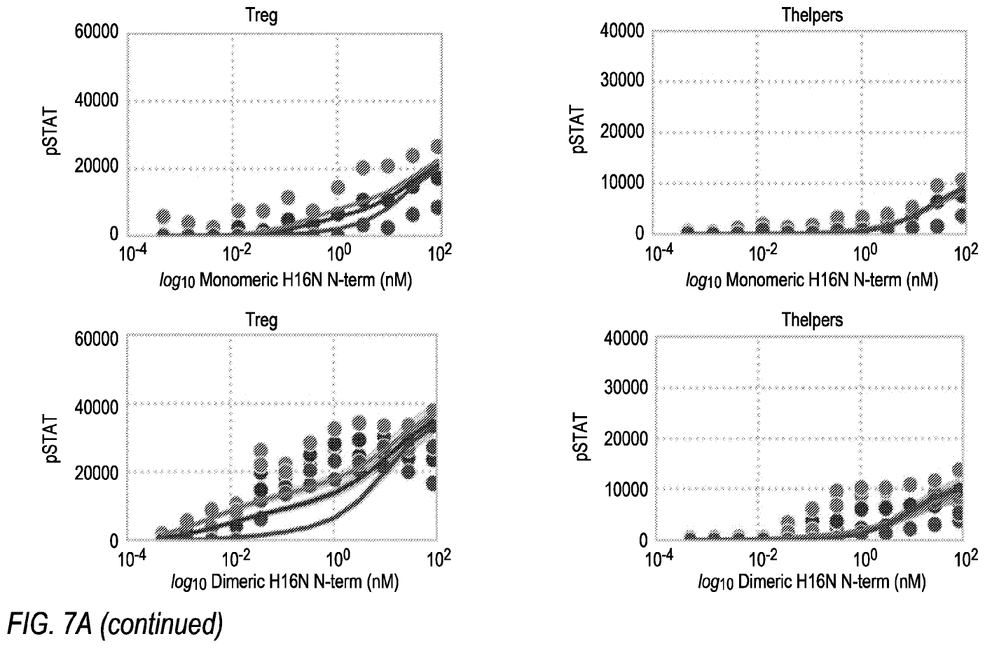

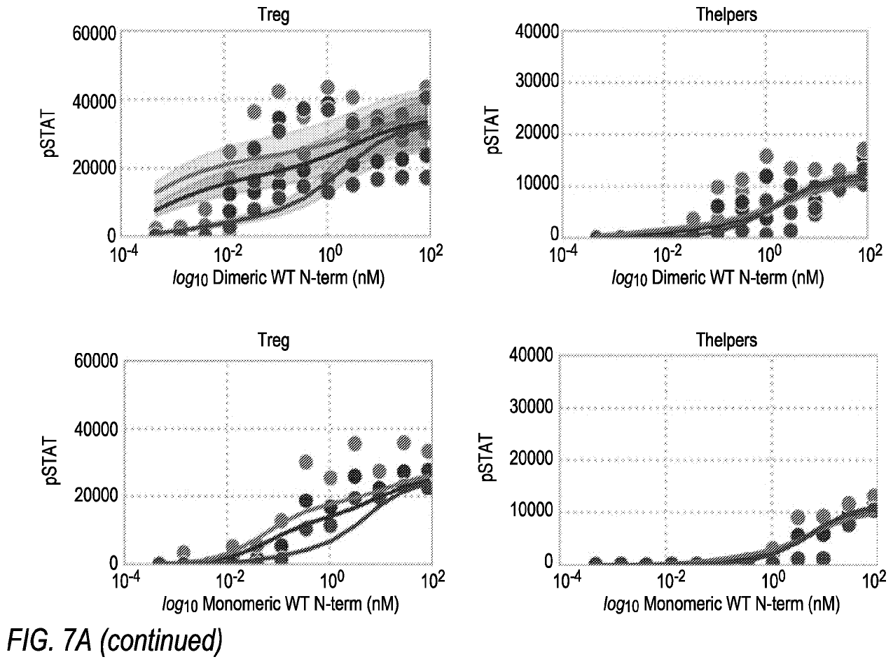

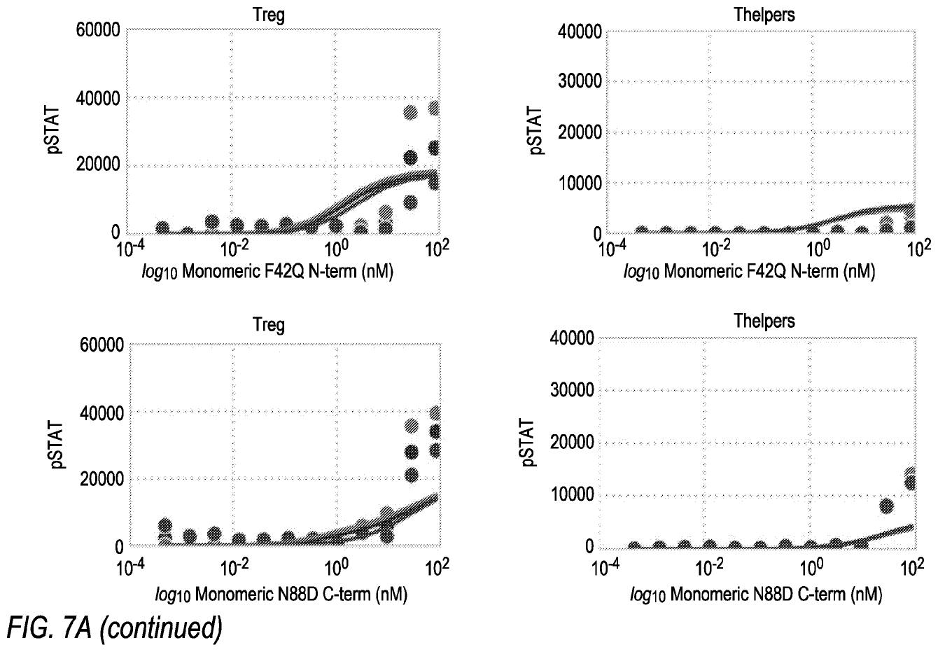

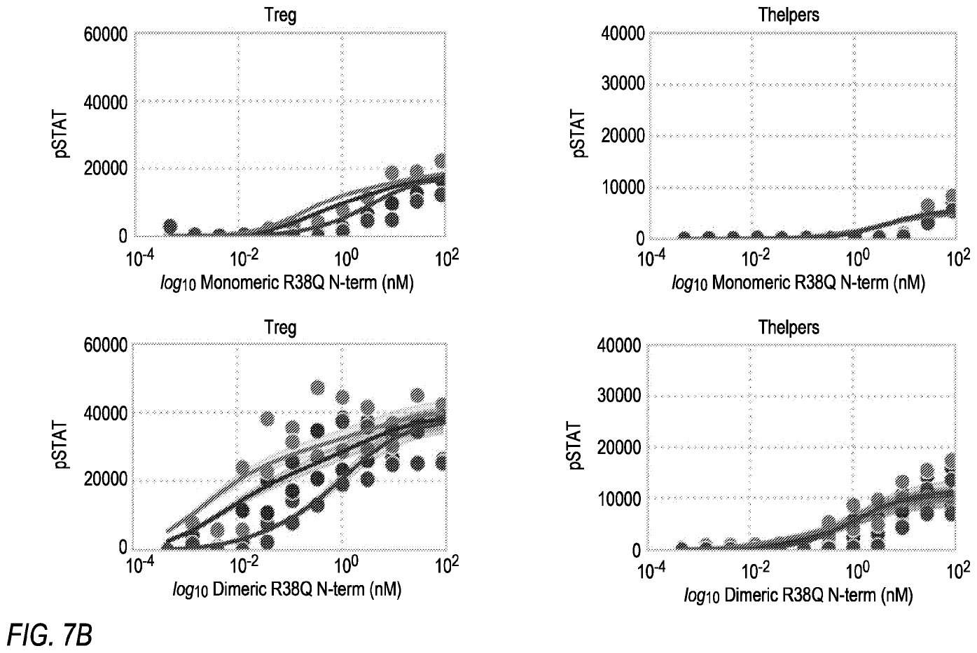

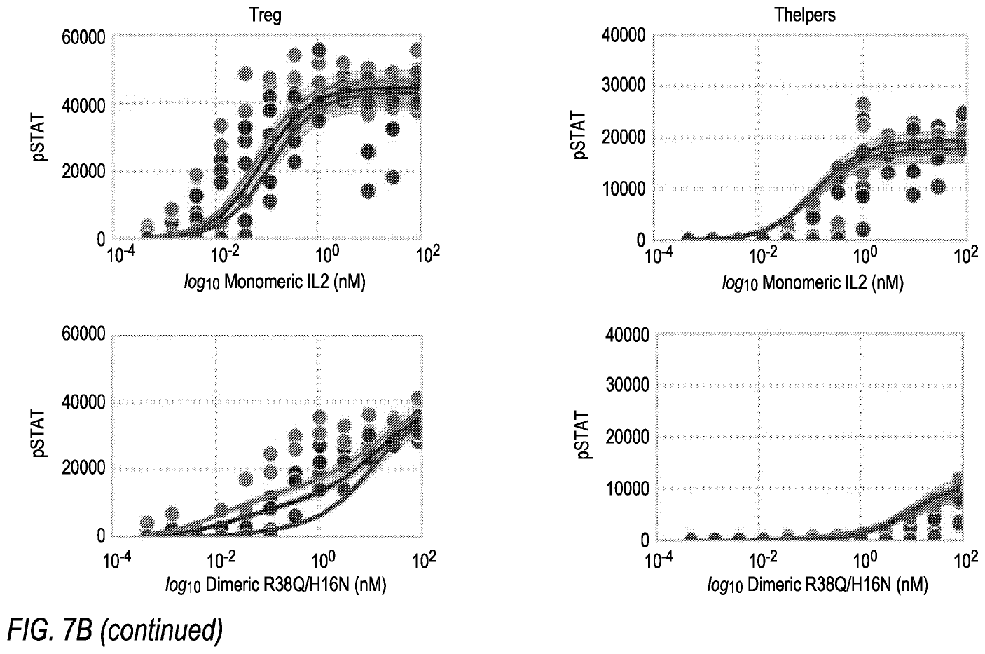

To further investigate the various determinants of signaling response, we selectively highlighted several dose response curves, each at 1 hour of treatment time (FIG. 1E-10). To first highlight the unique signaling behavior characterized by our various cell types, and their relationships with mutein format and valency, we isolated the responses of each cell type to various WT and affinity-altered monovalent and bivalent muteins (FIG. 1E-1H). We observed that WT IL-2 more potently activates all cell types beyond what is achieved by any Fc-fused muteins, suggesting that such conjugations uniformly reduce PBMC signaling response (FIG. 1D-1G). These responses also highlight the effects that affinity modulation has on cell-type specific responses; for example, R38Q-term, which binds to IL2Rβ with reduced affinity (FIG. 1B), was shown to induce significantly less potent responses across cell type. Valency also played a key role in determining signaling response, particularly in T_{reg} and Tₕₑₗₚₑᵣ populations, where selected bivalent muteins exhibited both increased sensitivity and potency in their signaling above their monovalent counterparts.

Next, we explored the effect of treatment time in determining signaling response (FIG. 1H-1K). At 1 hour valency, affinity, and the orientation of Fc fusions each play key roles in determining responses, particularly in T_{reg} populations; however, these characteristics which separate responses at 1 hour of treatment failed to result in distinct signaling responses after 4 hours of treatment for these ligands (FIG. 1H-1K). Thus, time and dynamics may play a key role in response to IL-2, an effect which may be mediated via receptor mediated endocytosis of IL-2 receptor subunits and transcriptional changes enacted by IL-2 signaling and STAT5 phosphorylation.

Finally, we visualized the effects of receptor abundance in determining IL-2 signaling response to H16N N-term (FIG. 1L-1O). Here, in T_{reg} populations with high amounts of IL2Rα were stimulated for 1 hour. We observed that both monovalent and bivalent H16N N-term induce potent signaling responses. However, in IL2Rαₗₒ T_{reg} populations, only the bivalent H16N ligand induced a significant response, highlighting the importance of ligand valency. However, the preserved signaling displayed in IL2Rαₗₒ T_{reg}s by bivalent H16N N-term did not hold in IL2Rαₗₒ Tₕₑₗₚₑᵣ populations.

Through both broad and isolated visualization of our expansive dataset of PBMC signaling response to IL-2 and IL-2 muteins, time, cell type, concentration, ligand affinity, valency, and Fc conjugation format all play unique roles in determining cellular response. These determinants interact in unique and often unintuitive manners.

Bivalent Fc-Cytokine Fusions have Distinct Cell Specificity but Shared Dynamics

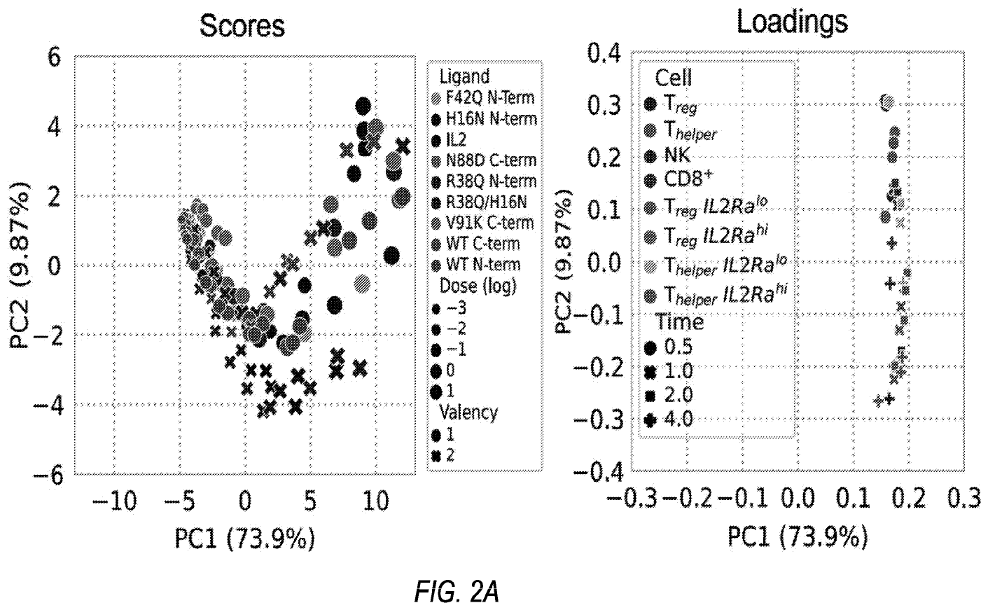

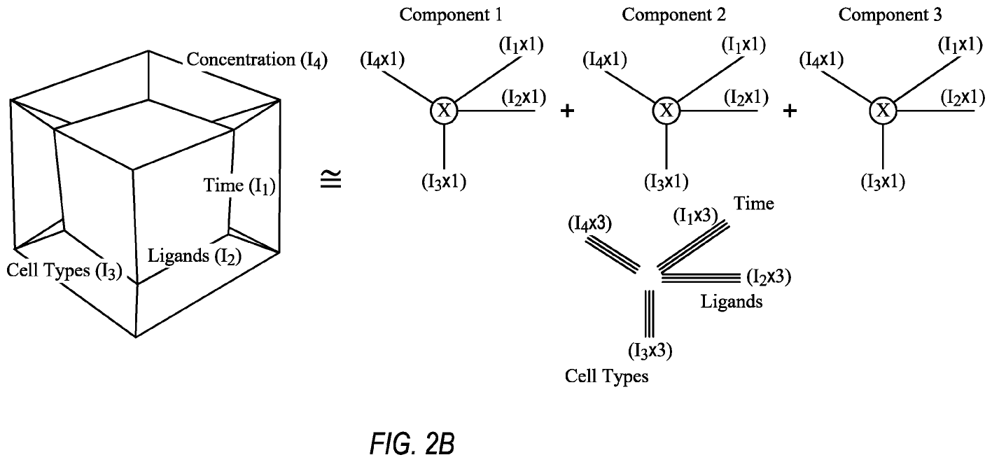

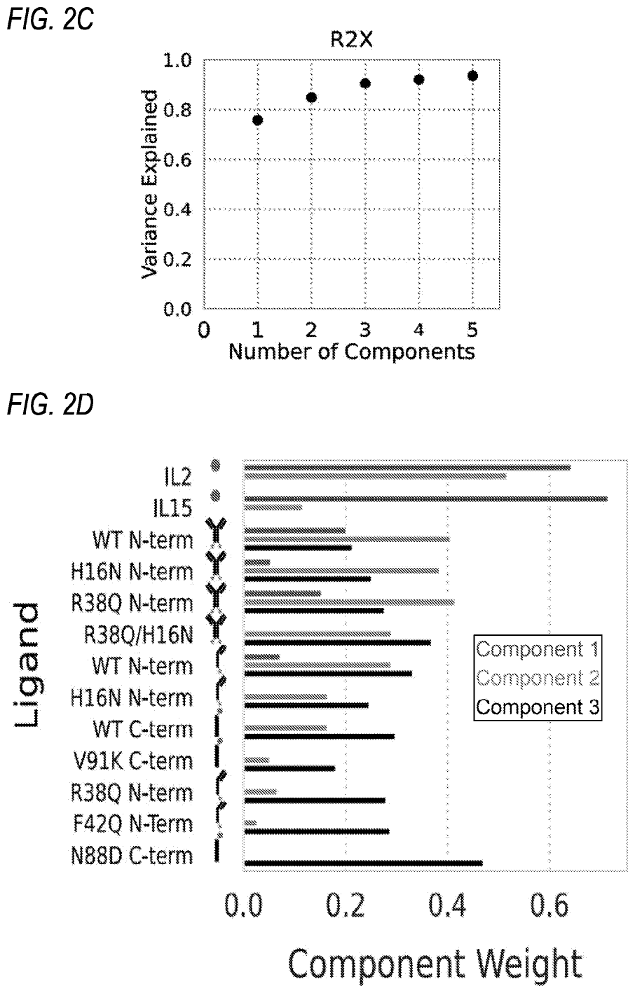

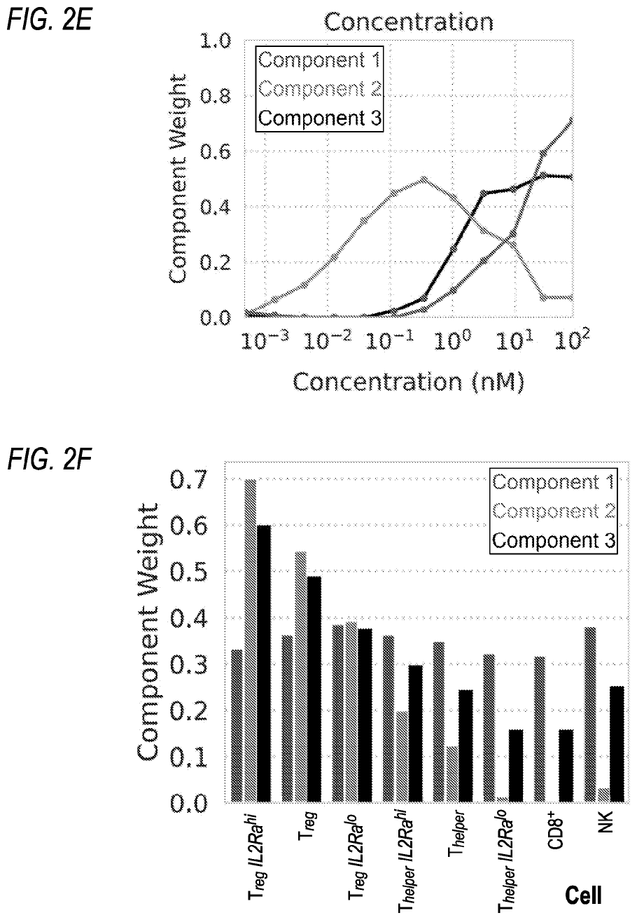

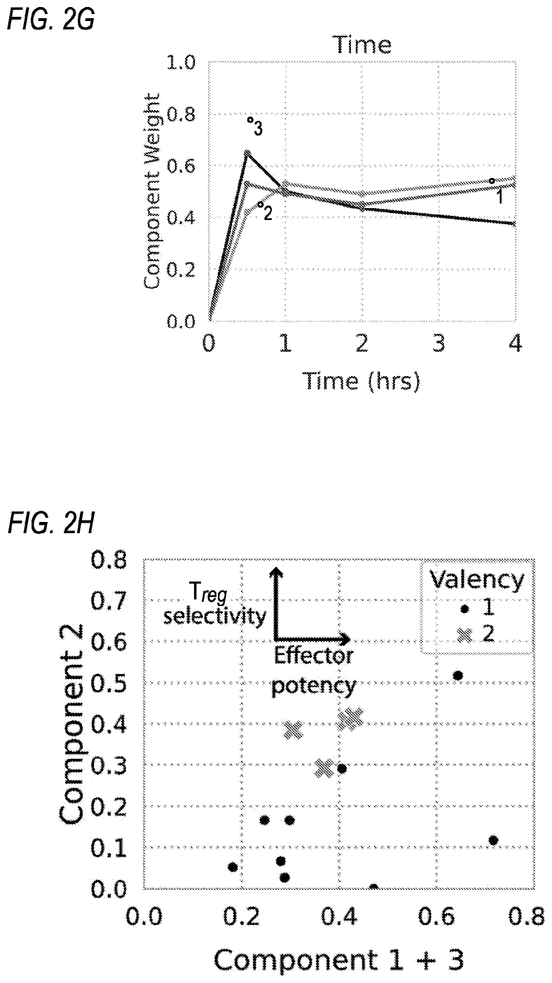

Exploring how dynamic responses vary across responding cell types and ligand treatments is challenging due to the multi-dimensional space of potential origins of contributory factors. Restricting ones' view to a single time point, cell type, or ligand concentration, as we showed in the previous results, provides only a slice of the picture. Dimensionality reduction is a natural solution, allowing the effects of these factors to be mapped to a shared space. However, when examining our signaling data by principal component analysis, we failed to deconvolute the effects of concentration from ligand, and time from cell type (FIG. 2A). To provide a more comprehensive view of response, we organized our profiling experiments into a four-dimensional tensor, wherein the position along each dimension represented the ligand used, concentration, treatment duration, or cell type. We then factored this data using non-negative canonical polyadic (CP) decomposition to derive factors summarizing the influence of each dimension (FIG. 2B). Three components explained roughly 90% of the variance within the dataset (FIG. 2C).

Factorization separated distinct response profiles into separate components and the effect of each dimension into separate factors. For instance, component 1 almost exclusively represented responses to wild-type cytokines (FIG. 2D), which were the only ligands not Fc-conjugated, showing a response primarily at high concentrations (FIG. 2E), with broad specificity (FIG. 2F) and a signaling profile that peaks at 30 minutes and then more rapidly decreases (FIG. 2G). An alternative way to interpret the factorization results is to compare profiles within a single dimension. For example, component 1 led to a less sustained profile of signaling response as compared to the other signaling patterns (FIG. 2G).

Remarkably, components 2 and 3 cleanly separated ligands conjugated in bivalent or monovalent forms, respectively (FIG. 2D). In fact, ligand valency was represented more prominently than differences in receptor affinity between muteins. Component 2 had uniquely high T_{reg} specificity (FIG. 2F) and was most represented at intermediate concentrations (FIG. 2E). Component 2 was also highly correlated with IL2Rα abundance in subsets of T_{reg} and Tₕₑₗₚₑᵣ cells, suggesting that bivalent molecule's specificity for T_{reg}s is mediated by their higher abundance of IL2Rα. Component 3 had a broad cell response (FIG. 2F) and increased monotonically with concentration (FIG. 2E). Despite these strong differences in other dimensions both components had nearly identical time dynamics (FIG. 2G). Furthermore, component 2, where T_{reg} response is highly weighted, was weighted higher for almost all bivalent muteins in comparison to components 1 and 3, which represented effector cell response (FIG. 2H). In total, these results indicated ligand valency as a critical determinant of IL-2 specificity, and that while Fc fusion subtly affected the specificity of response, monovalent and bivalent ligands had identical dynamics.

### Differences Among IL-2 Responses are Explained by a Simple Multivalent Binding Model

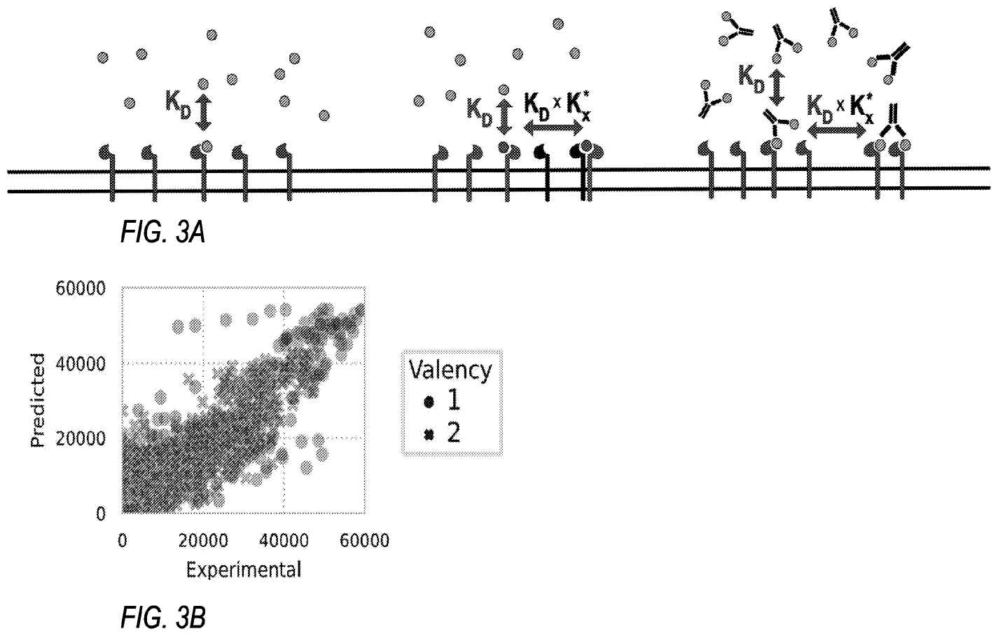

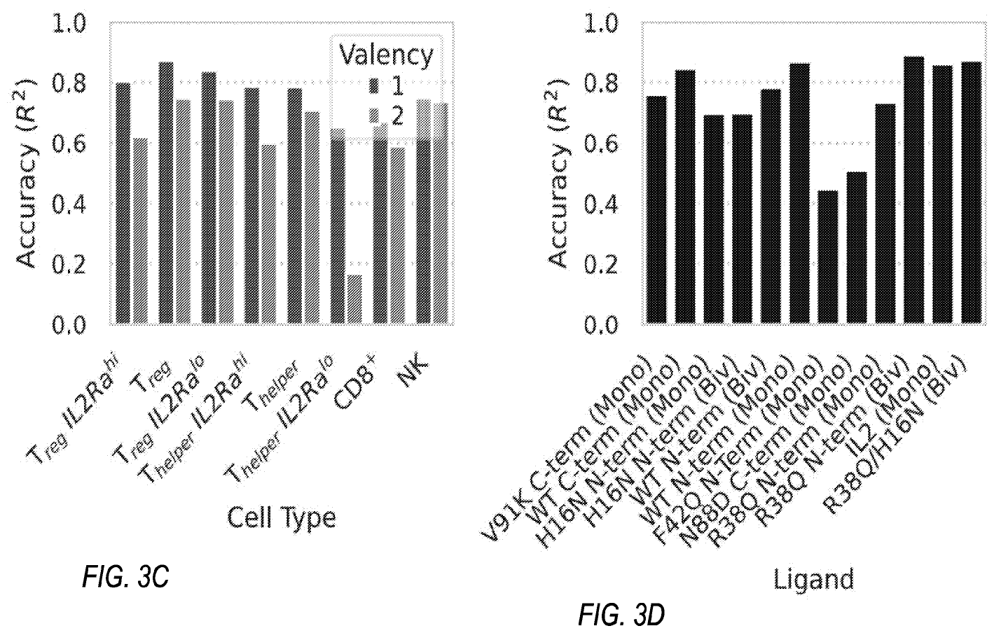

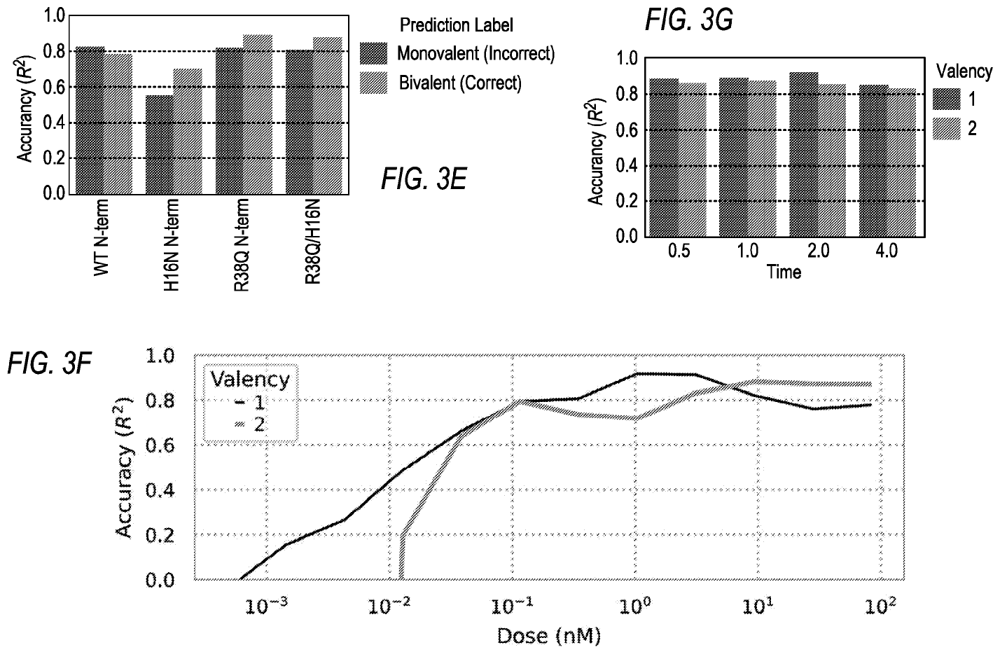

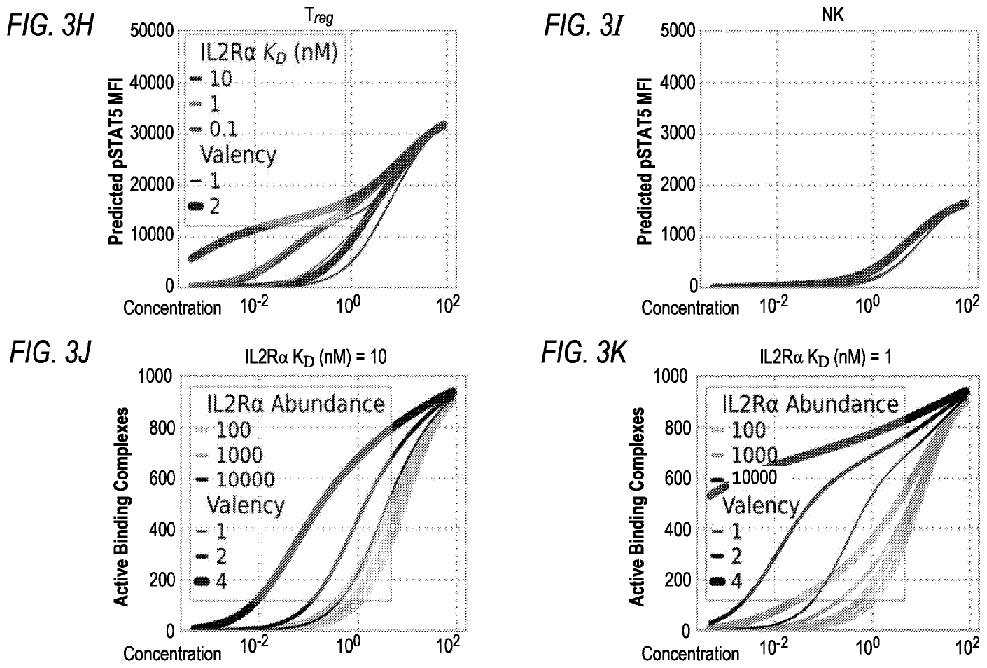

With the indication that T_{reg} specificity is enhanced by multivalency without changes to signaling dynamics, we sought to evaluate how well cell surface binding on its own could predict response. We employed a two-step, equilibrium, multivalent binding model to predict cellular response to IL-2 muteins by assuming that signaling response was proportional to the amount of active receptor-ligand complexes. See methods for specifics about how binding predictions were used to predict pSTAT5 response. We fit this model to our signaling profiling experiments and evaluated its concordance with the data. The only non-scaling fitting parameter, K*ₓ, had an optimum at 11.2×10⁻¹¹/cell, consistent with that seen for other receptor families. Overall, we observed remarkable consistency between predicted and observed response (R²=0.85; FIG. 3B). This high accuracy was maintained within subsets of the data including individual cell types and ligands (FIGS. 3C-3D). To verify our model's predictions were dependent on the valency of the molecule, we again fit the model enforcing that all ligands were monovalent. This significantly reduced model accuracy for many of the bivalent ligands (FIG. 3E), confirming that multivalent binding was critical in modeling signaling response.

To ensure that our model was not simply capturing a trend towards higher signaling with increasing concentration, we examined our model's accuracy within specific cytokine concentrations (FIG. 3F). Our model was unable to predict response at the lowest concentrations as these did not stimulate signaling and were dominated by experimental noise but increased in accuracy at concentrations where any signaling response was observed. Finally, we examined how our model's accuracy varied with time by fitting the model to each time point individually. As expected, given that longer treatments likely involve various compensatory signaling such as the degradation or increased transcription of IL-2 receptor subunits, we most accurately predicted initial responses (30 mins) with a slight decrease in accuracy over longer timescales (FIG. 3G). In total, multivalent cell surface binding showed quantitative agreement with the pattern of cell-type-specific responses to IL-2 muteins, supporting that the specificity enhancement of bivalency is derived from cell surface binding avidity effects.

To further characterize the dramatic effects multivalency and its interplay with binding affinity had on T_{reg} selectivity, we used our model to predict how T_{reg}s and NK cells would respond to bivalent IL-2 with varying IL2Rα affinities (FIG. 3H-3I). Here, our model again showed that T_{reg} response is strongly governed by IL2Rα affinities, and that these effects have an exceptionally strong relationship with valency, particularly at intermediate cytokine doses (FIG. 3H). Our model also shows that NK activation has very little relationship with ligand valency or IL2Rα affinity, demonstrating why multivalent ligands with strong IL2Rα binding avidities demonstrate exquisite T_{reg} selectivity. Finally, we used our model to demonstrate the intimate relationship which receptor abundance has on the binding of multivalent ligands (FIG. 3J-3K). Here, theoretical cell populations which express 104 IL2Rα molecules vary hugely in their response to multivalent IL-2 muteins, where cells expressing very few IL2Rα receptors barely alter in their response. In total, our model was able to demonstrate the unique relationship that multivalent ligands have with receptor abundances and visualize their ability to starkly separate responses in cells displaying variable amounts of identical receptors through avidity effects.

### Multivalency Provides a General Strategy for Further Enhanced Signaling Selectivity

Given that a simple binding model accurately predicted cell-type-specific responses to IL-2 and that bivalent, Fc-fused IL-2 muteins have favorable specificity properties, it was then sought to computationally explore to what extent multivalency might be a generally useful strategy. While monovalent ligand binding scales linearly with receptor abundance, multivalent ligands bind nonlinearly depending upon receptor abundance. Thus, multivalent ligands should be able to selectively target cells with uniquely high expression of certain γ_{c} family receptors.

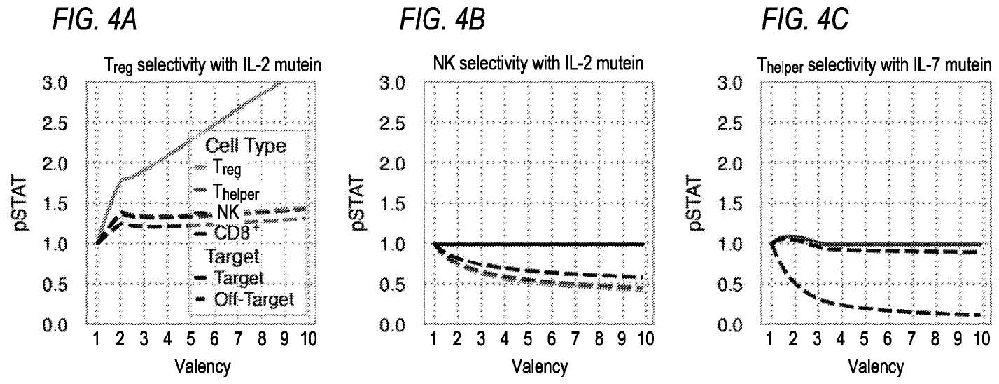

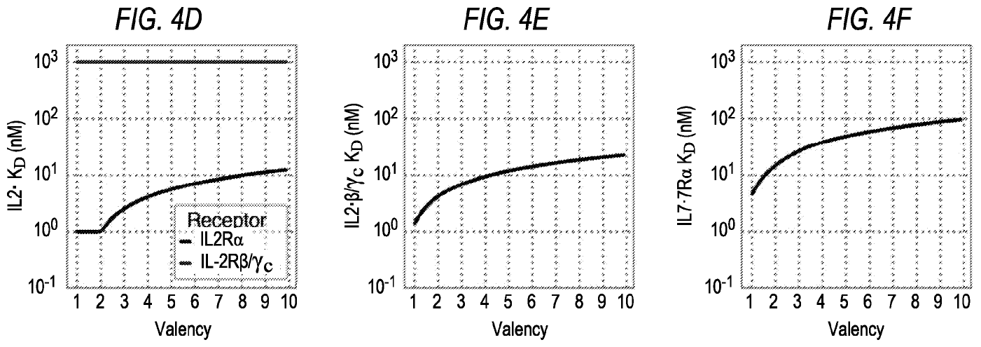

Valency enhancements are only apparent with coordinated changes in receptor-ligand binding affinities. Therefore, receptor affinities of simulated ligands were optimized while varying valency. IL-2 muteins of varying valency were first designed to obtain optimal T_{reg} specificity (FIG. 4A). As expected, ligand valency increased achievable selectivity past what was achievable using the bivalent cytokine format at any receptor affinity. Higher valency required reduced IL-2Rα affinities (FIG. 4B).

It was then explored whether IL-2 muteins lacking IL-2Rα binding could selectively target NK cells, based on their uniquely high expression of IL-2Rβ, with similar results; IL-2 muteins of higher valency were predicted to be highly selective for activation of NK cells, so long as IL-2Rβ/γ_{c} affinity was coordinately decreased (FIGS. 4C-4D). Finally, it was sought to explore Tₕₑₗₚₑᵣ-selective muteins of IL-7, as they express high amounts of IL-7Rα (FIG. 5I). It was again found that ligands of higher valency should achieve higher degrees of selectivity for these cells, but that the benefits of valency were less than the targeting of T_{reg}s or NK cells using IL-2 mutants because CD8⁺ T cells have more similar IL-7Rα amounts (FIG. 4E). These benefits were again found to be contingent on decreasing affinity of the IL-7 muteins for IL-7Rα at higher valency (FIG. 4F). In total, these results show that valency has unexplored potential for designing cytokines with enhanced therapeutic efficacy and reduced toxicity. They also show the potential benefit of the computational framework described above in guiding therapeutic development.

### Discussion

The present disclosure systematically explored how ligand properties determine signaling response across 13 IL-2 and IL-2 muteins, including both monovalent and bivalent Fc fusion variants. A tensor-based dimensionality reduction technique identified the patterns of changing response with ligand properties, revealing that multivalent cytokines have unique specificity but identical dynamics (FIG. 2). A multivalent binding model was able to accurately reproduce cell type specific response to IL-2 muteins with high accuracy, indicating that specificity is derived from surface binding avidity effects (FIG. 3). Through this model, it was found that cytokines of higher valency offer even greater cell type selectivity given corresponding affinity adjustments, which should translate to therapies of improved potency and reduced toxicity (FIG. 4).

The design of cell-type-selective ligands is complicated by the complex signaling processes by which they induce cellular signaling and ultimately response, which leads to a combinatorial explosion of potential ligand design objectives. The present disclosure serves to demonstrate the importance of the relatively unexplored axis of ligand development represented by the design of multivalent ligands. Multivalent ligands have several documented effects, including altered signal transduction, binding avidity, and pharmacokinetics or intracellular trafficking. While valency has been extensively explored as a means to introduce binding selectivity based on receptor density, how this effect interacts with the presence of multiple receptor species and cell signaling is surely more complex. The ability of the binding model described herein to accurately predict immune cell-type-specific response indicates that the cell-type-specific signaling profiles of these cytokines can still be understood as principally arising through receptor avidity effects at the cell surface (FIG. 3).

Results shown here may be used to guide the design of IL-2 muteins with high selectively for T_{reg}s, an important design criterion considered in the design of IL-2-based treatments for autoimmune diseases. By optimizing the predicted selectivity of high-valency ligands, it was shown that multivalency may be exploited to design more effective IL-2 based therapeutics for use in the clinical setting, where IL-2 based therapies have traditionally struggled (FIGS. 4A, 4B). Combined with the superior in vivo half-life conferred by Fc-fused IL-2 muteins, these multivalent therapeutics could potentially be used in an out-patient setting and require less frequent dosing. Furthermore, the superior selectivity offered by engineered multivalent ligands will likely further increase their in vivo half-lives, due to a reduction in receptor-mediated clearance by off-target populations. The present disclosure also demonstrated the potential benefit which multivalency may confer in the selective activation of NK cells, which could lead to similarly improved anti-cancer treatments. The approach disclosed herein may also be applied to engineer selectivity into other signaling pathways characterized by cell type pleiotropy, such as IL-4/IL-13 or TNF systems.

The approach disclosed herein effectively captures cell type specific responses to IL-2 and IL-2 muteins and can be readily applied to other well-studied signaling families. The model presented herein not only can be used to generate guidelines for the modulation of receptor-ligand interactions in conjunction with valency engineering to design more selective ligands, it can also predict specific receptor affinities are required to achieve these benefits (FIG. 4).

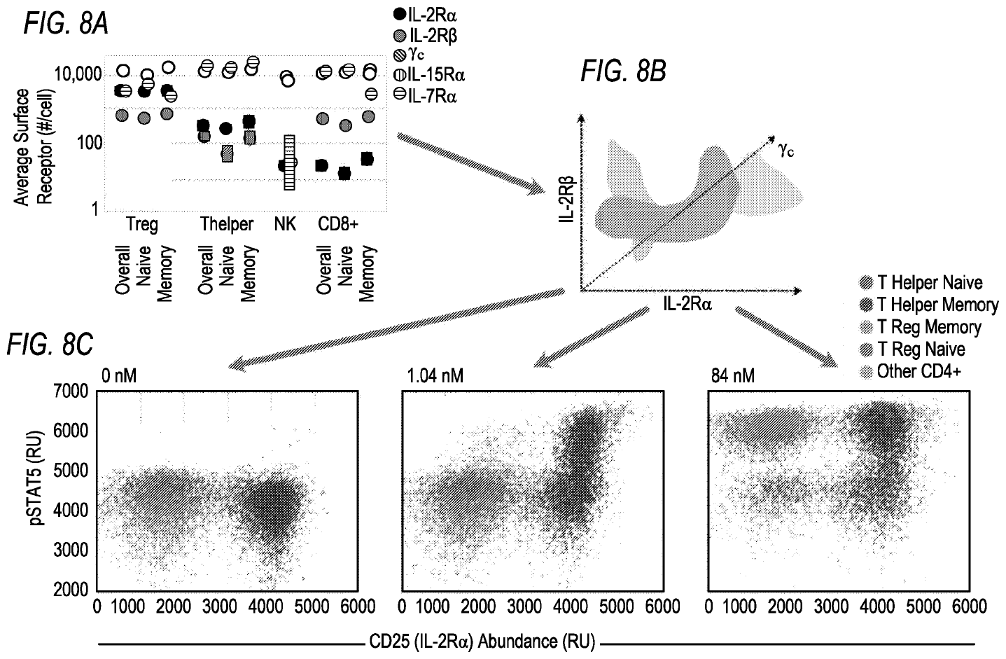

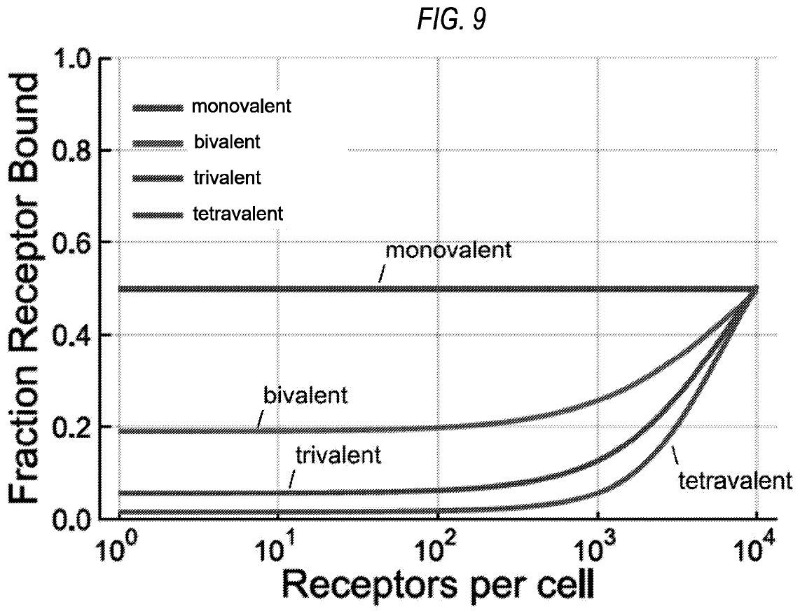

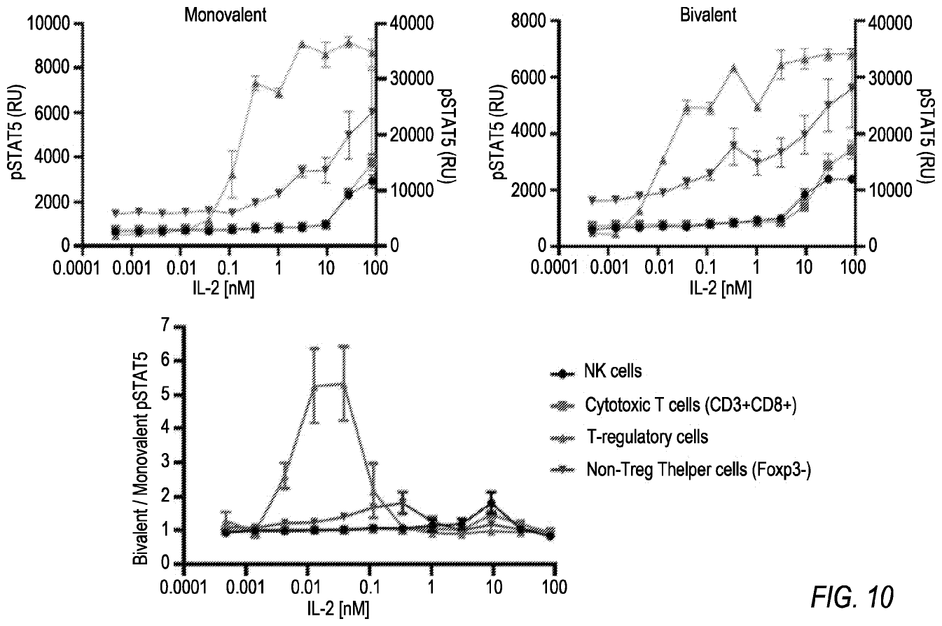

This example describes a route as disclosed herein toward obtaining cell-selective cytokines. While protein sequence changes can alter the relative affinity toward different receptors, they do not provide selectivity between cells expressing varying amounts of the same receptor (see FIG. 9). This quantitative, rather than qualitative, difference in receptor abundance is usually the case, such as with IL-2Rα expression in T_{reg}s relative to other cell types (see FIG. 8). The fact that on-target (e.g., T_{reg}) and off-target (e.g., Tₕₑₗₚₑᵣ) cells express the same receptors, just in subtly difference abundance, places upper limits on the selectivity possible with just affinity changes. In contrast, valency can bias binding toward more highly expressing cells over those with lower abundance (FIG. 9). In fact, this improved selectivity can already be seen when comparing a cytokine conjugated to an Fc in monovalent or bivalent format (FIG. 10). Higher valency, such as three or four cytokines tethered together, should offer even better cell selectivity (see FIG. 9). The superior selectivity of trivalent and tetravalent binders toward high antigen expressing cells has been observed with anti-tumor antibodies, but not with immune-modulatory cytokines. High-valency cytokines can be implemented through linker-based covalent coupling or through expression as a repeating peptide chain, either in free form or conjugated to an Fc for improved in vivo half-life. Valency-mediated improvements in selectivity should also be synergistic with affinity changes.

### Example 2

### Signaling Profiles of Tetravalent IL-2

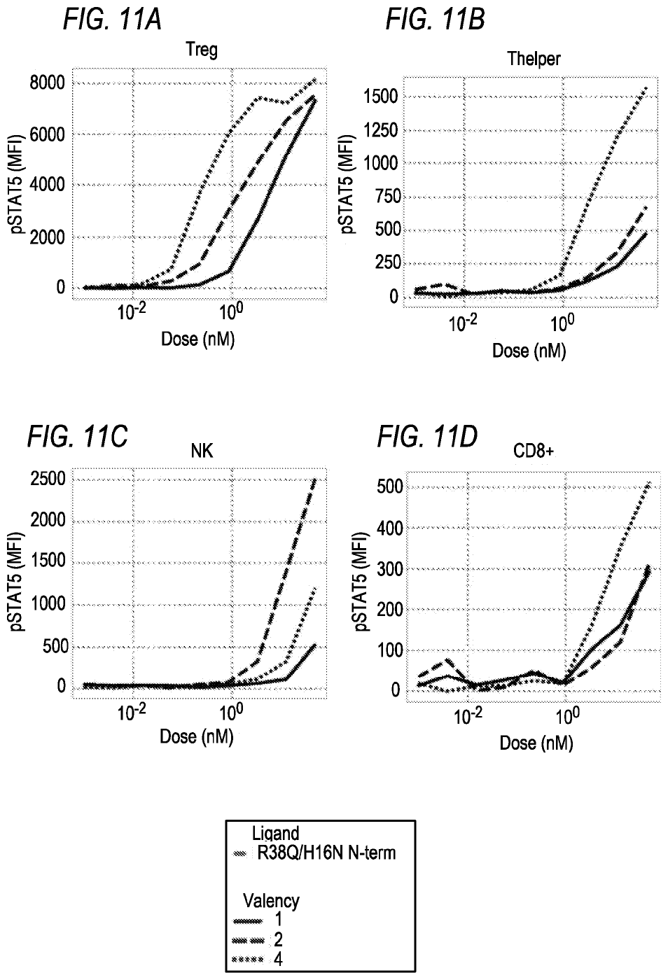

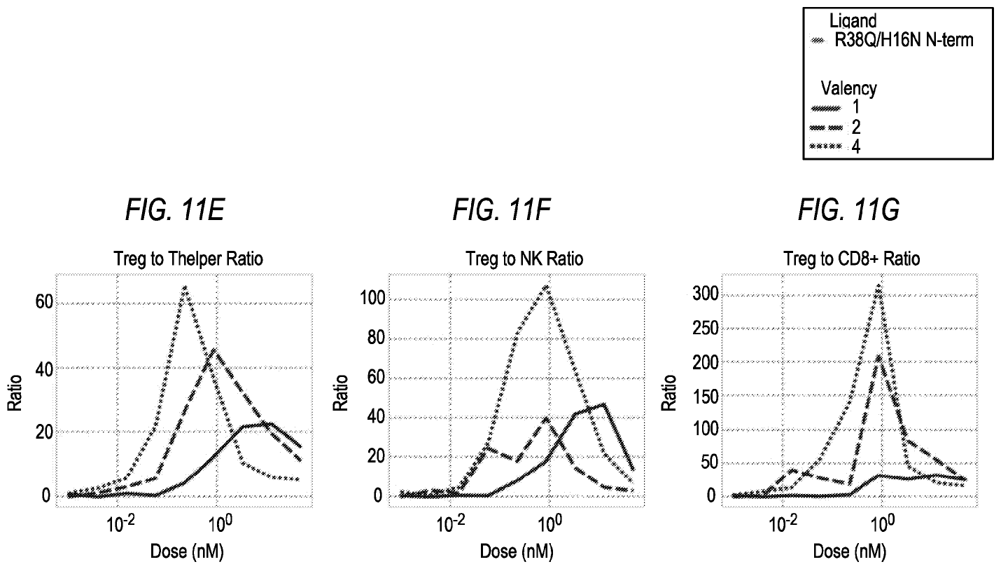

To experimentally validate whether increasing the valency of IL-2 muteins enhanced their cell-type selectivity, we designed and expressed monovalently, bivalently, and tetravalently Fc fused R38Q/H16N and used these muteins to stimulate human PBMCs from a single donor at 8 different cytokine concentrations. We separated the responses of the PBMCs by cell type and observed that tetravalent and, to a lesser extent, bivalent R38Q/H16N both drove increased selective STAT5 phosphorylation at lower doses in T_{reg} cells when compared to their monovalent counterparts (FIG. 11A). As predicted by our model (FIG. 5) the sensitivity of CD8+, NK, and Tₕₑₗₚₑᵣ cells were also modestly increased in response to multivalent ligands (FIGS. 11B-11D); however, when the ratio of T_{reg} response to the response of these off-target cell populations was calculated and plotted by dose, we found that the magnitude of the peak ratio of on- to off-target activation was greater and occurred at lower concentrations of cytokine in tetravalent and bivalent R38Q/H16N than the selectivity peak and location of monovalent R38Q/H16N (FIGS. 11E-11G). Thus, we concluded that increasing the valency of R38Q/H16N resulted in improvements in the cytokine's ability to target T_{reg}s selectivity.

### Methods

pSTAT5 Measurement of IL-2 and -15 Signaling in PBMCs

Cryopreserved PBMCs from healthy subjects were thawed to room temperature and slowly diluted with 9 mL pre-warmed RPMI-1640 medium (Corning, 10040CV) supplemented with 10% fetal bovine serum (FBS, VWR, 97068-091, lot #029K20) and Penicillin/Streptomycin (Gibco, 15140122). Media was removed, and cells were brought to 3×10⁶ cells/mL, distributed at 300,000 cells per well in a 96-well V-bottom plate, and allowed to recover 2 hrs at 37° C. in an incubator at 5% CO₂. IL-2 (Peprotech, 200-02-50 ug) and tetravalent IL-2 (expressed and purified as described below) were diluted in RPMI-1640 without FBS and added to the indicated concentrations. To measure pSTAT5, media was removed, and cells fixed in 100 μL of 4% paraformaldehyde (PFA, Election Microscopy Sciences, 15714) diluted in PBS for 15 mins at room temperature.

PFA was removed, and cells were gently suspended in 100 μL of cold methanol (−30° C.). Cells were stored overnight at −30° C. Cells were then washed twice with 0.1% bovine serum albumin (BSA, Sigma-Aldrich, B4287-25G) in PBS (PBSA), and stained 1 hr at room temperature in darkness using the antibody panel (Table 2) with 40 μL per well. Cells were then washed twice with 0.1% PBSA and resuspended in 150 μL PBSA per well. Cells were analyzed on a BD FACSCelesta flow cytometer using the High Throughput Sampler (HTS). Populations were gated as shown in FIG. 6, and the median pSTAT5 level was extracted for each population in each well.

TABLE 2
Antibody Panel

| Antibody | Identifier |
|---|---|
| Anti-CD25, BV786 (BD Biosciences) | CAT# 563701 Clone# M-A251 |
| | RRID: AB_2744338 |
| Anti-CD56, BV605 (Biolegend) | CAT# 318334 Clone# HCD56 |
| | RRID: AB_2561912 |
| Anti-CD8, FITC (Biolegend) | CAT# 301005 Clone# RPA-T8 |
| | RRID: AB_314123 |
| Anti-CD3, PE-Cy5 (Biolegend) | CAT# 300410 Clone# UCHT1 |
| | RRID: AB_314604 |
| Anti-pSTAT, R718 (BD Biosciences) | CAT# 566977 Clone# Clone 47 |
| | RRID: AB_2869984 |
| Anti-CD4, PE (Biolegend) | CAT# 300507 Clone# RPA-T4 |
| | RRID: AB_314075 |
| Anti-Foxp3, Pacific Blue (Biolegend) | CAT# 320216 Clone# 259D |
| | RRID: AB_2104902 |

### Recombinant Proteins

Plasmids containing tetravalent IL-2 inserts were generated by Twist Biosciences. Proteins were human IgG1 Fc-fused at the N- and C-terminus to mutant human IL-2 through a flexible (G4S)4 linker. C-terminal fusions omitted the C-terminal lysine residue of human IgG1. In bivalent variants, Fc mutations to prevent dimerization were introduced into the Fc sequence. Each human IL-2 fused via the 20 amino acid long linker to the Fc domain contained R38Q and H16N mutations. The R38Q and H16N mutations were introduced to reduce the IL-2's affinity with which it binds IL2Rβ. Plasmid DNA prepared by Maxi-Prep (Qiagen, 12162) was transfected into adherent HEK293T cells using Lipofectamine 3000 (Thermo-Fisher, L3000008) in 15 cm dishes in DMEM (Corning, 15017CV) supplemented with GlutaMax (Gibco, 35050061) and 10% FBS. Media was exchanged after 24 hrs with fresh DMEM supplemented with GlutaMax and 5% ultra-low IgG FBS (Thermo-Fisher, A3381901). Media was harvested after an additional 72 hours. Media was incubated in the presence of Protein A/G Plus Agarose resin (Santa Cruz Biotechnology, sc-2003) overnight. The following day, the media-resin mixture was centrifuged, and the supernatant discarded. Resin was washed with PBS until protein was no longer detected in supernatant by UV-Vis using the NanoDrop One Spectrophotometer (Thermo-Fisher, ND-ONE-W). IL-2 was eluted from resin using 0.1M glycine, pH 2.3, into 2M Tris-HCl, pH 8. IL-2 was then buffer exchanged into PBS for storage at −80° C. Concentration was determined using a custom IgG1 ELISA.

### Example 3

### Preparing Multivalent Cytokines

Various forms of multivalent cytokines may be prepared using fusions of a cytokine such as IL-2 with an Fc domain. In one embodiment, a single cytokine sequence is fused at each of the N and C termini of an Fc domain, forming a single-chain bivalent cytokine-Fc, which when dimerized forms a tetravalent cytokine. In another embodiment, a tandem sequence of two cytokines at the N-terminus and one at the C terminus forms a trivalent Fc fusion polypeptide, which when dimerized forms a hexavalent cytokine. In another embodiment, a tandem sequence of two cytokines expressed as a fusion polypeptide at both the N- and C-termini of Fe forms a tetravalent single-chain polypeptide, which when dimerized forms an octavalent cytokine.

The examples and embodiments herein incorporate the following components:

| | |
|---|---|
| MYRMQLLSCI ALSLALVTNS APTSSSTKKT QLQLEHLLLD LQMILNGINN | IL-2 |
| YKNPKLTRML TFKFYMPKKA TELKHLQCLE EELKPLEEVL NLAQSKNFHL | wild |
| RPRDLISNIN VIVLELKGSE TTFMCEYADE TATIVEFLNR WITFCQSIIS TLT | type |
| (SEQ ID NO: 1) | |
| APTSSSTKKT QLQLEHLLLD LQMILNGINN YKNPKLTRML TFKFYMPKKA | IL-2 |
| TELKHLQCLE EELKPLEEVL NLAQSKNFHL RPRDLISNIN VIVLELKGSE | without |
| TTFMCEYADE TATIVEFLNR WITFCQSIIS TLT (SEQ ID NO: 12) | signal |
| | sequence |
| MGEFAPTSSSTKKTQLQLEHLLLDLQMILNGINNYKNPKLTRMLTFKFYMPKKA | IL-2 |
| TELKH | super- |
| LQCLEEELKPLEEVLNLAQSKNFHFDPRDVVSNINVFVLELKGSETTFMCEYAD | kine |
| ETATIVEFLNRWITFCQSIISTLTH (SEQ ID NO: 76) | |
| GGGGGSGGGGSGGGGSGGGGS (SEQ ID NO: 13) | (G₄S)₄ |
| | linker |
| MFHVSFRYIFGLPPLILVLLPVASSDCDIEGKDG | IL-7 |
| KQYESVLMVSIDQLLDSMKEIGSNCLNNEFNFF | wild |
| KRHICDANKEGMFLFRAARKLRQFLKMNSTGD | type |
| FDLHLLKVSEGTTILLNCTGQVKGRKPAALGEA | |
| QPTKSLEENKSLKEQKKLNDLCFLKRLLQEIKTC | |
| WNKILMGTKEH (SEQ ID NO: 14) | |
| DCDIEGKDGKQYESVLMVSIDQLLDSMKEIGSNCLNNEFNFF | IL-7 |
| KRHICDANKEGMFLFRAARKLRQFLKMNSTGD | without |
| FDLHLLKVSEGTTILLNCTGQVKGRKPAALGEA | signal |
| QPTKSLEENKSLKEQKKLNDLCFLKRLLQEIKTC | sequence |
| WNKILMGTKEH (SEQ ID NO: 15) | |
| DKTHTCPPCPAPELLGGPSVFLFPPKPKDTL | Fc |
| MISRTPEVTCVVVDVSHEDPEVKFNWYVD | hinge |
| GVEVHNAKTKPREEQYNSTYRVVSVLTVL | region |
| HQDWLNGKEYKCKVSNKALPAPIEKTISKA | CH2/CH3, |
| KGQPREPQVYTLPPSRDELTKNQVSLTCLVKG | without |
| FYPSDIAVEWESNGQPENNYKTTPPVLDSDG | C-terminal |
| SFFLYSKLTVDKSRWQQGNVFSCSVMHEALHN | K |
| HYTQKSLSLSP (SEQ ID NO: 16) | |
| MGLTSQLLPPLFFLLACAGNFVHGHKCDITLQEIIKTLNSLTEQKTLCTELTVTDI | IL-4 |
| FAAS | wild |
| KNTTEKETFCRAATVLRQFYSHHEKDTRCLGATAQQFHRHKQLIRFLKRLDRNL | type |
| WGLAGL | |
| NSCPVKEANQSTLENFLERLKTIMREKYSKCSS (SEQ ID NO: 26) | |
| HKCDITLQEIIKTLNSLTEQKTLCTELTVTDIFAAS | IL-4 |
| KNTTEKETFCRAATVLRQFYSHHEKDTRCLGATAQQFHRHKQLIRFLKRLDRNL | without |
| WGLAGL | signal |
| NSCPVKEANQSTLENFLERLKTIMREKYSKCSS (SEQ ID NO: 27) | sequence |
| MLLAMVLTSALLLCSVAGQGCPTLAGILDINFLINKMQEDPASKCHCSANVTSC | IL-9 |
| LCLGIP | wild |
| SDNCTRPCFSERLSQMTNTTMQTRYPLIFSRVKKSVEVLKNNKCPYFSCEQPCN | type |
| QTTAGN | |
| ALTFLKSLLEIFQKEKMRGMRGKI (SEQ ID NO: 28) | |
| QGCPTLAGILDINFLINKMQEDPASKCHCSANVTSCLCLGIP | IL-9 |
| SDNCTRPCFSERLSQMTNTTMQTRYPLIFSRVKKSVEVLKNNKCPYFSCEQPCN | without |
| QTTAGN | signal |
| ALTFLKSLLEIFQKEKMRGMRGKI (SEQ ID NO: 29) | sequence |
| MRISKPHLRSISIQCYLCLLLNSHFLTEAGIHVFILGCFSAGLPKTEANWVNVISDL | IL-15 |
| KKI | wild |
| EDLIQSMHIDATLYTESDVHPSCKVTAMKCFLLELQVISLESGDASIHDTVENLII | type |
| LANN | |
| SLSSNGNVTESGCKECEELEEKNIKEFLQSFVHIVQMFINTS (SEQ ID NO: 30) | |
| GIHVFILGCFSAGLPKTEANWVNVISDLKKI | IL-15 |
| EDLIQSMHIDATLYTESDVHPSCKVTAMKCFLLELQVISLESGDASIHDTVENLII | without |
| LANN | signal |
| SLSSNGNVTESGCKECEELEEKNIKEFLQSFVHIVQMFINTS (SEQ ID NO: 31) | sequence |
| MRSSPGNMERIVICLMVIFLGTLVHKSSSQGQDRHMIRMRQLIDIVDQLKNYVN | IL-21 |
| DLVPEF | wild |
| LPAPEDVETNCEWSAFSCFQKAQLKSANTGNNERIINVSIKKLKRKPPSTNAGRR | type |
| QKHRL | |
| TCPSCDSYEKKPPKEFLERFKSLLQKMIHQHLSSRTHGSEDS (SEQ ID NO: 32) | |
| HKSSSQGQDRHMIRMRQLIDIVDQLKNYVNDLVPEF | IL-21 |
| LPAPEDVETNCEWSAFSCFQKAQLKSANTGNNERIINVSIKKLKRKPPSTNAGRR | without |
| QKHRL | signal |
| TCPSCDSYEKKPPKEFLERFKSLLQKMIHQHLSSRTHGSEDS (SEQ ID NO: 33) | sequence |

In one example of a multivalent IL-2 cytokine, a fusion polypeptide is expressed comprising the wild-type IL-2 sequence (SEQ ID NO: 1) at the N-terminus, a (G₄S)₄ linker (SEQ ID NO: 13), the CH2/C3 portion of Fe starting at the hinge region and lacking the C-terminal K (SEQ ID NO: 16), a (G₄S)₄ linker (SEQ ID NO: 13), and the IL-2 sequence without the signal peptide (SEQ ID NO: 12) at the C terminus. Such a bivalent, single-chain IL-2 Fc fusion is depicted in SEQ ID NO:9, and dimerization forms a tetravalent IL-2 (SEQ ID NO:23). For convenience, the aforementioned components of the sequence are shown with a line break between them, but the sequence is expressed as a single-chain polypeptide.

(SEQ ID NO: 9)
MYRMQLLSCIALSLALVTNSAPTSSSTKKTQLQLEHLLLDLQMILNGINNYKNPKLTRM
LTFKFYMPKKATELKHLQCLEEELKPLEEVLNLAQSKNFHLRPRDLISNINVIVLELKGSE
TTFMCEYADETATIVEFLNRWITFCQSIISTLT
GGGGSGGGGSGGGGSGGGGS
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSP
GGGGGSGGGGSGGGGSGGGGS
APTSSSTKKTQLQLEHLLLDLQMILNGINNYKNPKLTRMLTFKFYMPKKATELKHLQCL
EEELKPLEEVLNLAQSKNFHLRPRDLISNINVIVLELKGSETTFMCEYADETATIVEFLNR
WITFCQSIISTLT

In another example of a multivalent IL-2 cytokine, a fusion polypeptide is expressed comprising the wild-type IL-2 sequence (SEQ ID NO:1) at the N-terminus, a (G₄S)₄ linker (SEQ ID NO:13), an IL-2 sequence without the signal portion (SEQ ID NO:12), a (G₄S)₄ linker (SEQ ID NO:13), the CH2/C3 portion of Fc starting at the hinge region and lacking the C-terminal K (SEQ ID NO:16), a (G₄S)₄ linker (SEQ ID NO:13), the IL-2 sequence without the signal peptide (SEQ ID NO:12), a (G₄S)₄ linker (SEQ ID NO:13), and the IL-2 sequence without the signal peptide (SEQ ID NO:12) at the C terminus. Such a tetravalent, single-chain IL-2 Fc fusion is depicted in SEQ ID NO:10, and dimerization forms an octavalent IL-2 (SEQ ID NO:24). For convenience, the aforementioned components of the sequence are shown with a line break between them, but the sequence is expressed as a single-chain polypeptide.

(SEQ ID NO: 10)
MYRMQLLSCIALSLALVTNSAPTSSSTKKTQLQLEHLLLDLQMILNGINNYKNPKLTRM
LTFKFYMPKKATELKHLQCLEEELKPLEEVLNLAQSKNFHLRPRDLISNINVIVLELKGSE
TTFMCEYADETATIVEFLNRWITFCQSIISTLT
GGGGSGGGGSGGGGSGGGGS
APTSSSTKKTQLQLEHLLLDLQMILNGINNYKNPKLTRMLTFKFYMPKKATELKHLQCL
EEELKPLEEVLNLAQSKNFHLRPRDLISNINVIVLELKGSETTFMCEYADETATIVEFLNR
WITFCQSIISTLT
GGGGSGGGGSGGGGSGGGGS
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
GGGGSGGGGSGGGGSGGGGS
APTSSSTKKTQLQLEHLLLDLQMILNGINNYKNPKLTRMLTFKFYMPKKATELKHLQCL
EEELKPLEEVLNLAQSKNFHLRPRDLISNINVIVLELKGSETTFMCEYADETATIVEFLNR
WITFCQSIISTLT
GGGGSGGGGSGGGGSGGGGS
APTSSSTKKTQLQLEHLLLDLQMILNGINNYKNPKLTRMLTFKFYMPKKATELKHLQCL
EEELKPLEEVLNLAQSKNFHLRPRDLISNINVIVLELKGSETTFMCEYADETATIVEFLNR
WITFCQSIISTLT

In another example of a multivalent IL-7 cytokine, a fusion polypeptide is expressed comprising the wild-type IL-7 sequence (SEQ ID NO:14) at the N-terminus, a (G₄S)₄ linker (SEQ ID NO:13), the CH2/C3 portion of Fc starting at the hinge region and lacking the C-terminal K (SEQ ID NO:16), a (G₄S)₄ linker (SEQ ID NO:13), and the IL-7 sequence without the signal peptide (SEQ ID NO:15) at the C terminus. Such a bivalent, single-chain IL-7 Fc fusion is depicted in SEQ ID NO:11, and dimerization forms a tetravalent IL-7 (SEQ ID NO:25). For convenience, the aforementioned components of the sequence are shown with a line break between them, but the sequence is expressed as a single-chain polypeptide.

(SEQ ID NO: 11)
MFHVSFRYIFGLPPLILVLLPVASSDCDIEGKDGKQYESVLMVSIDQLLDSMKEIGSNCL
NNEFNFFKRHICDANKEGMFLFRAARKLRQFLKMNSTGDFDLHLLKVSEGTTILLNCTG
QVKGRKPAALGEAQPTKSLEENKSLKEQKKLNDLCFLKRLLQEIKTCWNKILMGTKEH
GGGGSGGGGSGGGGSGGGGS
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
GGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH.

In another example of a multivalent IL-7 cytokine, a fusion polypeptide is expressed comprising the wild-type IL-7 sequence (SEQ ID NO:14) at the N-terminus, a (G₄S)₄ linker (SEQ ID NO:13), an IL-7 sequence without the signal portion (SEQ ID NO:15), a (G₄S)₄ linker (SEQ ID NO:13), the CH2/C3 portion of Fc starting at the hinge region and lacking the C-terminal K (SEQ ID NO:16), a (G₄S)₄ linker (SEQ ID NO:13), the IL-7 sequence without the signal peptide (SEQ ID NO:15), a (G₄S)₄ linker (SEQ ID NO:13), and the IL-7 sequence without the signal peptide (SEQ ID NO:15) at the C terminus. Such a tetravalent, single-chain IL-7 Fc fusion is depicted in SEQ ID NO:744, and dimerization forms an octavalent IL-7 (SEQ ID NO:74). For convenience, the aforementioned components of the sequence are shown with a line break between them, but the sequence is expressed as a single-chain polypeptide.

(SEQ ID NO: 74)
MFHVSFRYIFGLPPLILVLLPVASSDCDIEGKDG
KQYESVLMVSIDQLLDSMKEIGSNCLNNEFNFF
KRHICDANKEGMFLFRAARKLRQFLKMNSTGD
FDLHLLKVSEGTTILLNCTGQVKGRKPAALGEA
QPTKSLEENKSLKEQKKLNDLCFLKRLLQEIKTC
WNKILMGTKEH
GGGGGSGGGGSGGGGSGGGGS
DCDIEGKDGKQYESVLMVSIDQLLDSMKEIGSNCLNNEFNFF
KRHICDANKEGMFLFRAARKLRQFLKMNSTGD
FDLHLLKVSEGTTILLNCTGQVKGRKPAALGEA
QPTKSLEENKSLKEQKKLNDLCFLKRLLQEIKTC
WNKILMGTKEH
GGGGGSGGGGSGGGGSGGGGS
DKTHTCPPCPAPELLGGPSVFLFPPKPKDTL
MISRTPEVTCVVVDVSHEDPEVKFNWYVD
GVEVHNAKTKPREEQYNSTYRVVSVLTVL
HQDWLNGKEYKCKVSNKALPAPIEKTISKA
KGQPREPQVYTLPPSRDELTKNQVSLTCLVKG
FYPSDIAVEWESNGQPENNYKTTPPVLDSDG
SFFLYSKLTVDKSRWQQGNVFSCSVMHEALHN
HYTQKSLSLSP
GGGGGSGGGGSGGGGSGGGGS
DCDIEGKDGKQYESVLMVSIDQLLDSMKEIGSNCLNNEFNFF
KRHICDANKEGMFLFRAARKLRQFLKMNSTGD
FDLHLLKVSEGTTILLNCTGQVKGRKPAALGEA
QPTKSLEENKSLKEQKKLNDLCFLKRLLQEIKTC
WNKILMGTKEH
GGGGGSGGGGSGGGGSGGGGS
DCDIEGKDGKQYESVLMVSIDQLLDSMKEIGSNCLNNEFNFF
KRHICDANKEGMFLFRAARKLRQFLKMNSTGD
FDLHLLKVSEGTTILLNCTGQVKGRKPAALGEA
QPTKSLEENKSLKEQKKLNDLCFLKRLLQEIKTC
WNKILMGTKEH.

Alternate cross-linking methods. The chemistry for selectively reducing and then covalently conjugating to certain cysteines within an IgG has been extensively characterized for antibody-drug conjugate applications. See, for example, Agarwal et al., Site-Specific Antibody-Drug Conjugates: The Nexus of Bioorthogonal Chemistry, Protein Engineering, and Drug Development, Bioconjugate Chem 2015; 26:176-192. For example, a bivalent Fc-fused IL-2 is treated with tris(2-carboxyethyl)phosphine (TCEP) as a partial reducing agent to break the two disulfide bonds between the IgG chains. A bifunctional PEG-maleimide cross-linker is then used to create a covalent bond with two of the Fc-fusion complexes. This results in a four-valent complex. Properly formed complexes are separated from larger polymers or remaining single bivalent complexes by size exclusion chromatography.

### Example 4

### Multivalent, Single Polypeptide Chain Cytokines

Both the N- and C-terminus of IL-2 are located close to one another on the same side of the cytokine-receptor complex. As a result, a tetravalent polymer chain of IL-2 (SEQ ID NO:2) can be expressed as a single polypeptide by placing four copies of the IL-2 sequence (SEQ ID NO:1) in series, linked by (G4S)ₙ linkers. In one embodiment, the linker length is chosen from n=4, 8, or 12. In one embodiment, appropriate value of n is chosen based on the shortest length showing full potency, as reduced signaling response is likely indicative of steric hindrance.

In one embodiment, SEQ ID NO:2 is an amino acid sequence of a tetravalent IL-2 linked together by three (G4S)₄ linkers. The N-terminal IL-2 includes the signal sequence (SEQ ID NO:1) but the subsequent IL-2 do not (SEQ ID NO:12). For convenience, the ensuing sequences' components are shown with a line break between them, but each depicted sequence is expressed as a single-chain polypeptide.

(TETRAVALENT IL-2)
SEQ ID NO: 2
MYRMQLLSCI ALSLALVTNS APTSSSTKKT QLQLEHLLLD LQMILNGINN
YKNPKLTRML TFKFYMPKKA TELKHLQCLE EELKPLEEVL NLAQSKNFHL
RPRDLISNIN VIVLELKGSE TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT

In a similar fashion as described above, additional fusion polyvalent IL-2 sequences may be prepared.

SEQ ID NO: 4 (TRIVALENT IL-2):
MYRMQLLSCI ALSLALVINS APTSSSTKKT QLQLEHLLLD LQMILNGINN
YKNPKLTRML TFKFYMPKKA TELKHLQCLE EELKPLEEVL NLAQSKNFHL
RPRDLISNIN VIVLELKGSE TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
SEQ ID NO: 5 (PENTAVALENT IL-2):
MYRMQLLSCI ALSLALVTNS APTSSSTKKT QLQLEHLLLD LQMILNGINN
YKNPKLTRML TFKFYMPKKA TELKHLQCLE EELKPLEEVL NLAQSKNFHL
RPRDLISNIN VIVLELKGSE TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
SEQ ID NO: 6 (HEXAVALENT IL-2):
MYRMQLLSCI ALSLALVTNS APTSSSTKKT QLQLEHLLLD LQMILNGINN
YKNPKLTRML TFKFYMPKKA TELKHLQCLE EELKPLEEVL NLAQSKNFHL
RPRDLISNIN VIVLELKGSE TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
SEQ ID NO: 7 (HEPTAVALENT IL-2):
MYRMQLLSCI ALSLALVTNS APTSSSTKKT QLQLEHLLLD LQMILNGINN
YKNPKLTRML TFKFYMPKKA TELKHLQCLE EELKPLEEVL NLAQSKNFHL
RPRDLISNIN VIVLELKGSE TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
SEQ ID NO: 8 (OCTAVALENT IL-2):
MYRMQLLSCI ALSLALVINS APTSSSTKKT QLQLEHLLLD LQMILNGINN
YKNPKLTRML TFKFYMPKKA TELKHLQCLE EELKPLEEVL NLAQSKNFHL
RPRDLISNIN VIVLELKGSE TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT
GGGGS GGGGS GGGGS GGGGS
APTSSSTKKT QLQLEHLLLD LQMILNGINN YKNPKLTRML TFKFYMPKKA
TELKHLQCLE EELKPLEEVL NLAQSKNFHL RPRDLISNIN VIVLELKGSE
TTFMCEYADE TATIVEFLNR WITFCQSIIS TLT

In a similar fashion, single-chain IL-7 multivalent cytokines can be prepared.

SEQ ID NO: 22 (TRIVALENT IL-7):
MFHVSFRYIFGLPPLILVLLPVASSDCDIEGKDGKQYESVLMVSIDQLLDSMKEIGSNCLN
NEFNFFKRHICDANKEGMFLFRAARKLRQFLKMNSTGDFDLHLLKVSEGTTILLNCTGQ
VKGRKPAALGEAQPTKSLEENKSLKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
SEQ ID NO: 17 (TETRAVALENT IL-7):
MFHVSFRYIFGLPPLILVLLPVASSDCDIEGKDGKQYESVLMVSIDQLLDSMKEIGSNCLN
NEFNFFKRHICDANKEGMFLFRAARKLRQFLKMNSTGDFDLHLLKVSEGTTILLNCTGQ
VKGRKPAALGEAQPTKSLEENKSLKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
SEQ ID NO: 18 (PENTAVALENT IL-7):
MFHVSFRYIFGLPPLILVLLPVASSDCDIEGKDGKQYESVLMVSIDQLLDSMKEIGSNCLN
NEFNFFKRHICDANKEGMFLFRAARKLRQFLKMNSTGDFDLHLLKVSEGTTILLNCTGQ
VKGRKPAALGEAQPTKSLEENKSLKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
SEQ ID NO: 19 (HEXAVALENT IL-7):
MFHVSFRYIFGLPPLILVLLPVASSDCDIEGKDGKQYESVLMVSIDQLLDSMKEIGSNCLN
NEFNFFKRHICDANKEGMFLFRAARKLRQFLKMNSTGDFDLHLLKVSEGTTILLNCTGQ
VKGRKPAALGEAQPTKSLEENKSLKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
SEQ ID NO: 20 (HEPTAVALENT IL-7):
MFHVSFRYIFGLPPLILVLLPVASSDCDIEGKDGKQYESVLMVSIDQLLDSMKEIGSNCLN
NEFNFFKRHICDANKEGMFLFRAARKLRQFLKMNSTGDFDLHLLKVSEGTTILLNCTGQ
VKGRKPAALGEAQPTKSLEENKSLKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
SEQ ID NO: 21 (OCTAVALENT IL-7):
MFHVSFRYIFGLPPLILVLLPVASSDCDIEGKDGKQYESVLMVSIDQLLDSMKEIGSNCLN
NEFNFFKRHICDANKEGMFLFRAARKLRQFLKMNSTGDFDLHLLKVSEGTTILLNCTGQ
VKGRKPAALGEAQPTKSLEENKSLKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
GGGGGSGGGGSGGGGSGGGGS
DCDIEGKDGKQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH

Further IL-2 multimers may be prepared by adding further subunits.

### Example 5

### Additional Multivalent Cytokines

Following the guidance provided herein on preparing multivalent cytokines of IL-2 and IL-7, analogous methods and constructs are prepared using IL4, IL-9, IL-15 or IL-21, for the purposes described herein.

### IL-4

For example, single-chain IL-4 multivalent cytokines may be prepared with a sequence comprising (from N- to C-terminus): the full length sequence of IL-4 (SEQ ID NO:26) and two or more iterations of a linker such as SEQ ID NO: 13 and the sequence of IL-4 without the signal sequence (SEQ ID NO:27).

| | |
|---|---|
| TRIVALENT | MGLTSQLLPPLFFLLACAGNFVHGHKCDITLQEIIKTLNSLTEQKTLCT |
| IL-4 | ELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | (SEQ ID NO: 34) |
| TETRALAVENT | MGLTSQLLPPLFFLLACAGNFVHGHKCDITLQEIIKTLNSLTEQKTLCT |
| IL-4 | ELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | (SEQ ID NO: 35) |
| PENTAVALENT | MGLTSQLLPPLFFLLACAGNFVHGHKCDITLQEIIKTLNSLTEQKTLCT |
| IL-4 | ELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | (SEQ ID NO: 36) |
| HEXAVALENT | MGLTSQLLPPLFFLLACAGNFVHGHKCDITLQEIIKTLNSLTEQKTLCT |
| IL-4 | ELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | (SEQ ID NO: 37) |
| HEPTAVALENT | MGLTSQLLPPLFFLLACAGNFVHGHKCDITLQEIIKTLNSLTEQKTLCT |
| IL-4 | ELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | (SEQ ID NO: 38) |
| OCTAVALENT | MGLTSQLLPPLFFLLACAGNFVHGHKCDITLQEIIKTLNSLTEQKTLCT |
| IL-4 | ELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFL |
| | KRLDRNLWGLAGLNSCPVKEANQSTLENFLERLKTIMREKYSKCSS |
| | (SEQ ID NO: 39) |

In another embodiment, a bivalent fusion polypeptide is expressed comprising the wild-type IL-4 sequence (SEQ ID NO:26) at the N-terminus, a (G₄S)₄ linker (SEQ ID NO: 13), the CH2/C3 portion of Fe starting at the hinge region and lacking the C-terminal K (SEQ ID NO: 16), a (G₄S)₄ linker (SEQ ID NO: 13), and the IL-4 sequence without the signal peptide (SEQ ID NO:27) at the C terminus. Such a bivalent, single-chain IL-4 Fc fusion is depicted in SEQ ID NO:58, and disulfide dimerization forms a tetravalent IL-4 (SEQ ID NO:59). For convenience, the aforementioned components of the sequence are shown with a line break between them, but the sequence is expressed as a single-chain polypeptide.

In another example of a multivalent IL-4 cytokine, a fusion polypeptide is expressed comprising the wild-type IL-4 sequence (SEQ ID NO:26) at the N-terminus, a (G₄S)4 linker (SEQ ID NO: 13), an IL-4 sequence without the signal portion (SEQ ID NO:27), a (G₄S)₄ linker (SEQ ID NO:13), the CH2/C3 portion of starting at the hinge region and lacking the C-terminal K (SEQ ID NO:16), a (G₄)₄ linker (SEQ ID NO: 13), the IL-4 sequence without the signal peptide (SEQ ID NO:27), a (G₄S)₄ linker (SEQ ID NO: 13), and the IL-4 sequence without the signal peptide (SEQ ID NO:27) at the C terminus. Such a tetravalent, single-chain IL-4 Fc fusion is depicted in SEQ ID NO: 60, and disulfide dimerization forms an octavalent IL-4 (SEQ ID NO: 61). For convenience, the aforementioned components of the sequence are shown with a line break between them, but the sequence is expressed as a single-chain polypeptide.

| | |
|---|---|
| BIVALENT IL-4 | MGLTSQLLPPLFFLLACAGNFVHGHKCDITLQEIIKTLNSLTEQKTLCTELT |
| (forms tetravalent | VTDIFAAS |
| IL-4 by disulfide | KNTTEKETFCRAATVLRQFYSHHEKDTRCLGATAQQFHRHKQLIRFLKRL |
| dimerization of | DRNLWGLAGL |
| Fc regions; SEQ | NSCPVKEANQSTLENFLERLKTIMREKYSKCSS |
| ID NO: 59) | GGGGGSGGGGSGGGGSGGGGS |
| | DKTHTCPPCPAPELLGGPSVFLFPPKPKDTL |
| | MISRTPEVTCVVVDVSHEDPEVKFNWYVD |
| | GVEVHNAKTKPREEQYNSTYRVVSVLTVL |
| | HQDWLNGKEYKCKVSNKALPAPIEKTISKA |
| | KGQPREPQVYTLPPSRDELTKNQVSLTCLVKG |
| | FYPSDIAVEWESNGQPENNYKTTPPVLDSDG |
| | SFFLYSKLTVDKSRWQQGNVFSCSVMHEALHN |
| | HYTQKSLSLSP |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFLKRL |
| | DRNLWGLAGL |
| | NSCPVKEANQSTLENFLERLKTIMREKYSKCSS (SEQ ID NO: 58) |
| TETRAVALENT | MGLTSQLLPPLFFLLACAGNFVHGHKCDITLQEIIKTLNSLTEQKTLCTELT |
| IL-4 | VTDIFAAS |
| (forms octavalent | KNTTEKETFCRAATVLRQFYSHHEKDTRCLGATAQQFHRHKQLIRFLKRL |
| IL-2 by disulfide | DRNLWGLAGL |
| dimerization of | NSCPVKEANQSTLENFLERLKTIMREKYSKCSS |
| Fc regions; SEQ | GGGGGSGGGGSGGGGSGGGGS |
| ID NO: 61) | HKCDITLQEIIKTLNSLTEQKTLCTELTVTDIFAAS |
| | KNTTEKETFCRAATVLRQFYSHHEKDTRCLGATAQQFHRHKQLIRFLKRL |
| | DRNLWGLAGL |
| | NSCPVKEANQSTLENFLERLKTIMREKYSKCSS |
| | GGGGGSGGGGSGGGGSGGGGS |
| | DKTHTCPPCPAPELLGGPSVFLFPPKPKDTL |
| | MISRTPEVTCVVVDVSHEDPEVKFNWYVD |
| | GVEVHNAKTKPREEQYNSTYRVVSVLTVL |
| | HQDWLNGKEYKCKVSNKALPAPIEKTISKA |
| | KGQPREPQVYTLPPSRDELTKNQVSLTCLVKG |
| | FYPSDIAVEWESNGQPENNYKTTPPVLDSDG |
| | SFFLYSKLTVDKSRWQQGNVFSCSVMHEALHN |
| | HYTQKSLSLSP |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFLKRL |
| | DRNLWGLAGL |
| | NSCPVKEANQSTLENFLERLKTIMREKYSKCSS |
| | GGGGGSGGGGSGGGGSGGGGS |
| | HKCDITLQEIIKTLNSLTEQKTLCTELTVTDIFAAS |
| | KNTTEKETFCRAATVLRQFYSHHEKDTRCLGATAQQFHRHKQLIRFLKRL |
| | DRNLWGLAGL |
| | NSCPVKEANQSTLENFLERLKTIMREKYSKCSS (SEQ ID NO: 60) |

### IL-9

For example, single-chain IL-4 multivalent cytokines may be prepared with a sequence comprising (from N- to C-terminus): the full length sequence of IL-4 (SEQ ID NO:26) and two or more iterations of a linker such as SEQ ID NO: 13 and the sequence of IL-4 without the signal sequence (SEQ ID NO:27).

| | |
|---|---|
| TRIVALENT | MLLAMVLTSALLLCSVAGQGCPTLAGILDINFLINKMQEDPASKC |
| IL-9 | HCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI (SEQ ID NO: 40) |
| TETRALAVENT | MLLAMVLTSALLLCSVAGQGCPTLAGILDINFLINKMQEDPASKC |
| IL-9 | HCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI (SEQ ID NO: 41) |
| PENTAVALENT | MLLAMVLTSALLLCSVAGQGCPTLAGILDINFLINKMQEDPASKC |
| IL-9 | HCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI (SEQ ID NO: 42) |
| HEXAVALENT | MLLAMVLTSALLLCSVAGQGCPTLAGILDINFLINKMQEDPASKC |
| IL-9 | HCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI (SEQ ID NO: 43) |
| HEPTAVALENT | MLLAMVLTSALLLCSVAGQGCPTLAGILDINFLINKMQEDPASKC |
| IL-9 | HCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI (SEQ ID NO: 44) |
| OCTAVALENT | MLLAMVLTSALLLCSVAGQGCPTLAGILDINFLINKMQEDPASKC |
| IL-9 | HCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKNNKCP |
| | YFSCEQPCNQTTAGN |
| | ALTFLKSLLEIFQKEKMRGMRGKI (SEQ ID NO: 45) |

In another embodiment, a bivalent fusion polypeptide is expressed comprising the wild-type IL-9 sequence (SEQ ID NO:28) at the N-terminus, a (G₄S)₄ linker (SEQ ID NO:13), the CH2/C3 portion of Fc starting at the hinge region and lacking the C-terminal K (SEQ ID NO:16), a (G₄S)₄ linker (SEQ ID NO:13), and the IL-9 sequence without the signal peptide (SEQ ID NO:29) at the C terminus. Such a bivalent, single-chain IL-9 Fc fusion is depicted in SEQ ID NO:62, and disulfide dimerization forms a tetravalent IL-9 (SEQ ID NO:63). For convenience, the aforementioned components of the sequence are shown with a line break between them, but the sequence is expressed as a single-chain polypeptide.

In another example of a multivalent IL-9 cytokine, a fusion polypeptide is expressed comprising the wild-type IL-9 sequence (SEQ ID NO:28) at the N-terminus, a (G₄S)₄ linker (SEQ ID NO:13), an IL-9 sequence without the signal portion (SEQ ID NO:29), a (G₄S)₄ linker (SEQ ID NO:13), the CH2/C3 portion of Fc starting at the hinge region and lacking the C-terminal K (SEQ ID NO:16), a (G₄S)₄ linker (SEQ ID NO:13), the IL-9 sequence without the signal peptide (SEQ ID NO:29), a (G₄S)₄ linker (SEQ ID NO:13), and the IL-9 sequence without the signal peptide (SEQ ID NO:29) at the C terminus. Such a tetravalent, single-chain IL-9 Fc fusion is depicted in SEQ ID NO:64, and disulfide dimerization forms an octavalent IL-9 (SEQ ID NO:65). For convenience, the aforementioned components of the sequence are shown with a line break between them, but the sequence is expressed as a single-chain polypeptide.

| | |
|---|---|
| BIVALENT IL-9 | MLLAMVLTSALLLCSVAGQGCPTLAGILDINFLIN |
| (forms tetravalent | KMQEDPASKCHCSANVTSCLCLGIP |
| IL-9 by disulfide | SDNCTRPCFSERLSQMTNTTMQTRYPLIFS |
| dimerization of | RVKKSVEVLKNNKCPYFSCEQPCNQTTAGN |
| Fc regions; SEQ | ALTFLKSLLEIFQKEKMRGMRGKI |
| ID NO: 63) | GGGGGSGGGGSGGGGSGGGGS |
| | DKTHTCPPCPAPELLGGPSVFLFPPKPKDTL |
| | MISRTPEVTCVVVDVSHEDPEVKFNWYVD |
| | GVEVHNAKTKPREEQYNSTYRVVSVLTVL |
| | HQDWLNGKEYKCKVSNKALPAPIEKTISKA |
| | KGQPREPQVYTLPPSRDELTKNQVSLTCLVKG |
| | FYPSDIAVEWESNGQPENNYKTTPPVLDSDG |
| | SFFLYSKLTVDKSRWQQGNVFSCSVMHEALHN |
| | HYTQKSLSLSP |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKN |
| | NKCPYFSCEQPCNQTTAGN |
| | ALTFLKSLLEIFQKEKMRGMRGKI (SEQ ID NO: 62) |
| TETRAVALENT | MLLAMVLTSALLLCSVAGQGCPTLAGILDINFLIN |
| IL-9 | KMQEDPASKCHCSANVTSCLCLGIP |
| (forms octavalent | SDNCTRPCFSERLSQMTNTTMQTRYPLIFS |
| IL-2 by disulfide | RVKKSVEVLKNNKCPYFSCEQPCNQTTAGN |
| dimerization of | ALTFLKSLLEIFQKEKMRGMRGKI |
| Fc regions; SEQ | GGGGGSGGGGSGGGGSGGGGS |
| ID NO: 65) | QGCPTLAGILDINFLINKMQEDPASKCHCSANVTSCLCLGIP |
| | SDNCTRPCFSERLSQMTNTTMQTRYPLIFSRVKKSVEVLKN |
| | NKCPYFSCEQPCNQTTAGN |
| | ALTFLKSLLEIFQKEKMRGMRGKI |
| | GGGGGSGGGGSGGGGSGGGGS |
| | DKTHTCPPCPAPELLGGPSVFLFPPKPKDTL |
| | MISRTPEVTCVVVDVSHEDPEVKFNWYVD |
| | GVEVHNAKTKPREEQYNSTYRVVSVLTVL |
| | HQDWLNGKEYKCKVSNKALPAPIEKTISKA |
| | KGQPREPQVYTLPPSRDELTKNQVSLTCLVKG |
| | FYPSDIAVEWESNGQPENNYKTTPPVLDSDG |
| | SFFLYSKLTVDKSRWQQGNVFSCSVMHEALHN |
| | HYTQKSLSLSP |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLKN |
| | NKCPYFSCEQPCNQTTAGN |
| | ALTFLKSLLEIFQKEKMRGMRGKI |
| | GGGGGSGGGGSGGGGSGGGGS |
| | QGCPTLAGILDINFLINKMQEDPASKCHCSANVTSCLCLGIP |
| | SDNCTRPCFSERLSQMTNTTMQTRYPLIFSRVKKSVEVLK |
| | NNKCPYFSCEQPCNQTTAGN |
| | ALTFLKSLLEIFQKEKMRGMRGKI (SEQ ID NO: 64) |

### IL-15

For example, single-chain IL-4 multivalent cytokines may be prepared with a sequence comprising (from N- to C-terminus): the full length sequence of IL-15 (SEQ ID NO:26) and two or more iterations of a linker such as SEQ ID NO: 13 and the sequence of IL-15 without the signal sequence (SEQ ID NO:27).

| | |
|---|---|
| TRIVALENT | MRISKPHLRSISIQCYLCLLLNSHFLTEAGIHVFILGCFSAGLPKTEA |
| IL-15 | NWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | (SEQ ID NO: 46) |
| TETRALAVENT | MRISKPHLRSISIQCYLCLLLNSHFLTEAGIHVFILGCFSAGLPKTEA |
| IL-15 | NWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | (SEQ ID NO: 47) |
| PENTAVALENT | MRISKPHLRSISIQCYLCLLLNSHFLTEAGIHVFILGCFSAGLPKTEA |
| IL-15 | NWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | (SEQ ID NO: 48) |
| HEXAVALENT | MRISKPHLRSISIQCYLCLLLNSHFLTEAGIHVFILGCFSAGLPKTEA |
| IL-15 | NWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | (SEQ ID NO: 49) |
| HEPTAVALENT | MRISKPHLRSISIQCYLCLLLNSHFLTEAGIHVFILGCFSAGLPKTEA |
| IL-15 | NWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | (SEQ ID NO: 50) |
| OCTAVALENT | MRISKPHLRSISIQCYLCLLLNSHFLTEAGIHVFILGCFSAGLPKTEA |
| IL-15 | NWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQVISLESGDAS |
| | IHDTVENLIILANN |
| | SLSSNGNVTESGCKECEELEEKNIKEFLQSFVHIVQMFINTS |
| | (SEQ ID NO: 51) |

In another embodiment, a bivalent fusion polypeptide is expressed comprising the wild-type IL-15 sequence (SEQ ID NO:30′) at the N-terminus, a (G₄S)₄ linker (SEQ ID NO:13′), the CH2/C3 portion of Fc starting at the hinge region and lacking the C-terminal K (SEQ ID NO:16), a (G₄S)₄ linker (SEQ ID NO:13), and the IL-15 sequence without the signal peptide (SEQ ID NO:31) at the C terminus. Such a bivalent, single-chain IL-15 Fc fusion is depicted in SEQ ID NO:66, and disulfide dimerization forms a tetravalent IL-15 (SEQ ID NO:67). For convenience, the aforementioned components of the sequence are shown with a line break between them, but the sequence is expressed as a single-chain polypeptide.

In another example of a multivalent IL-15 cytokine, a fusion polypeptide is expressed comprising the wild-type IL-15 sequence (SEQ ID NO:30) at the N-terminus, a (G₄S)₄ linker (SEQ ID NO:13), an IL-15 sequence without the signal portion (SEQ ID NO:31), a (G₄S)₄ linker (SEQ ID NO:13), the CH2/C3 portion of Fc starting at the hinge region and lacking the C-terminal K (SEQ ID NO:31), a (G₄S)₄ linker (SEQ ID NO:13), the IL-15 sequence without the signal peptide (SEQ ID NO:31), a (G₄S)₄ linker (SEQ ID NO:13), and the IL-15 sequence without the signal peptide (SEQ ID NO:31) at the C terminus. Such a tetravalent, single-chain IL-15 Fc fusion is depicted in SEQ ID NO:68, and disulfide dimerization forms an octavalent IL-15 (SEQ ID NO:69). For convenience, the aforementioned components of the sequence are shown with a line break between them, but the sequence is expressed as a single-chain polypeptide.

| | |
|---|---|
| BIVALENT IL-15 | MRISKPHLRSISIQCYLCLLLNSHFLTEAGIHVFILG |
| (forms tetravalent | CFSAGLPKTEANWVNVISDLKKI |
| IL-15 by | EDLIQSMHIDATLYTESDVHPSCKVTAMKCFLLEL |
| disulfide | QVISLESGDASIHDTVENLIILANN |
| dimerization of | SLSSNGNVTESGCKECEELEEKNIKEFLQSFVHIVQMFINTS |
| Fc regions; SEQ | GGGGGSGGGGSGGGGSGGGGS |
| ID NO: 67) | DKTHTCPPCPAPELLGGPSVFLFPPKPKDTL |
| | MISRTPEVTCVVVDVSHEDPEVKFNWYVD |
| | GVEVHNAKTKPREEQYNSTYRVVSVLTVL |
| | HQDWLNGKEYKCKVSNKALPAPIEKTISKA |
| | KGQPREPQVYTLPPSRDELTKNQVSLTCLVKG |
| | FYPSDIAVEWESNGQPENNYKTTPPVLDSDG |
| | SFFLYSKLTVDKSRWQQGNVFSCSVMHEALHN |
| | HYTQKSLSLSP |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQV |
| | ISLESGDASIHDTVENLIILANN |
| | SLSSNGNVTESGCKECEELEEKNIKEFLQSFVHIV |
| | QMFINTS (SEQ ID NO: 66) |
| TETRAVALENT | MRISKPHLRSISIQCYLCLLLNSHFLTEAGIHVFILG |
| IL-15 | CFSAGLPKTEANWVNVISDLKKI |
| (forms octavalent | EDLIQSMHIDATLYTESDVHPSCKVTAMKCFLLEL |
| IL-2 by disulfide | QVISLESGDASIHDTVENLIILANN |
| dimerization of | SLSSNGNVTESGCKECEELEEKNIKEFLQSFVHIVQMFINTS |
| Fc regions; SEQ | GGGGGSGGGGSGGGGSGGGGS |
| ID NO: 69) | GIHVFILGCFSAGLPKTEANWVNVISDLKKI |
| | EDLIQSMHIDATLYTESDVHPSCKVTAMKCFLLELQV |
| | ISLESGDASIHDTVENLIILANN |
| | SLSSNGNVTESGCKECEELEEKNIKEFLQSFVHIV |
| | QMFINTS |
| | GGGGGSGGGGSGGGGSGGGGS |
| | DKTHTCPPCPAPELLGGPSVFLFPPKPKDTL |
| | MISRTPEVTCVVVDVSHEDPEVKFNWYVD |
| | GVEVHNAKTKPREEQYNSTYRVVSVLTVL |
| | HQDWLNGKEYKCKVSNKALPAPIEKTISKA |
| | KGQPREPQVYTLPPSRDELTKNQVSLTCLVKG |
| | FYPSDIAVEWESNGQPENNYKTTPPVLDSDG |
| | SFFLYSKLTVDKSRWQQGNVFSCSVMHEALHN |
| | HYTQKSLSLSP |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQV |
| | ISLESGDASIHDTVENLIILANN |
| | SLSSNGNVTESGCKECEELEEKNIKEFLQSFVHIV |
| | QMFINTS |
| | GGGGGSGGGGSGGGGSGGGGS |
| | GIHVFILGCFSAGLPKTEANWVNVISDLKKI |
| | EDLIQSMHIDATLYTESDVHPSCKVTAMKCFLLELQV |
| | ISLESGDASIHDTVENLIILANN |
| | SLSSNGNVTESGCKECEELEEKNIKEFLQSFVHIV |
| | QMFINTS (SEQ ID NO: 68) |

### IL-21

For example, single-chain IL-21 multivalent cytokines may be prepared with a sequence comprising (from N- to C-terminus): the full length sequence of IL-21 (SEQ ID NO:32) and two or more iterations of a linker such as SEQ ID NO: 13 and the sequence of IL-21 without the signal sequence (SEQ ID NO:33).

| | |
|---|---|
| TRIVALENT | MRSSPGNMERIVICLMVIFLGTLVHKSSSQGQDRHMIRMRQLIDIV |
| IL-21 | DQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS (SEQ |
| | ID NO: 52) |
| TETRALAVENT | MRSSPGNMERIVICLMVIFLGTLVHKSSSQGQDRHMIRMRQLIDIV |
| IL-21 | DQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS (SEQ |
| | ID NO: 53) |
| PENTAVALENT | MRSSPGNMERIVICLMVIFLGTLVHKSSSQGQDRHMIRMRQLIDIV |
| IL-21 | DQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS (SEQ |
| | ID NO: 54) |
| HEXAVALENT | MRSSPGNMERIVICLMVIFLGTLVHKSSSQGQDRHMIRMRQLIDIV |
| IL-21 | DQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS (SEQ |
| | ID NO: 55) |
| HEPTAVALENT | MRSSPGNMERIVICLMVIFLGTLVHKSSSQGQDRHMIRMRQLIDIV |
| IL-21 | DQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS (SEQ |
| | ID NO: 56) |
| OCTAVALENT | MRSSPGNMERIVICLMVIFLGTLVHKSSSQGQDRHMIRMRQLIDIV |
| IL-21 | DQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKRK |
| | PPSTNAGRRQKHRL |
| | TCPSCDSYEKKPPKEFLERFKSLLQKMIHQHLSSRTHGSEDS (SEQ |
| | ID NO: 57) |

In another embodiment, a bivalent fusion polypeptide is expressed comprising the wild-type IL-21 sequence (SEQ ID NO:32) at the N-terminus, a (G₄S)₄ linker (SEQ ID NO:13), the CH2/C3 portion of Fe starting at the hinge region and lacking the C-terminal K (SEQ ID NO: 16), a (G₄S)₄ linker (SEQ ID NO: 13), and the IL-21 sequence without the signal peptide (SEQ ID NO:33) at the C terminus. Such a bivalent, single-chain IL-21 Fc fusion is depicted in SEQ ID NO:70, and disulfide dimerization forms a tetravalent IL-21 (SEQ ID NO:71). For convenience, the aforementioned components of the sequence are shown with a line break between them, but the sequence is expressed as a single-chain polypeptide.

In another example of a multivalent IL-21 cytokine, a fusion polypeptide is expressed comprising the wild-type IL-21 sequence (SEQ ID NO:32) at the N-terminus, a (G₄S)₄ linker (SEQ ID NO:13), an IL-21 sequence without the signal portion (SEQ ID NO:33), a (G₄S)₄ linker (SEQ ID NO:13), the CH2/C3 portion of Fc starting at the hinge region and lacking the C-terminal K (SEQ ID NO:33), a (G₄S)₄ linker (SEQ ID NO:13), the IL-21 sequence without the signal peptide (SEQ ID NO:33), a (G₄S)₄ linker (SEQ ID NO:13), and the IL-21 sequence without the signal peptide (SEQ ID NO:33) at the C terminus. Such a tetravalent, single-chain IL-21 Fc fusion is depicted in SEQ ID NO:72, and disulfide dimerization forms an octavalent IL-21 (SEQ ID NO:73). For convenience, the aforementioned components of the sequence are shown with a line break between them, but the sequence is expressed as a single-chain polypeptide.

| | |
|---|---|
| BIVALENT IL-21 | MRSSPGNMERIVICLMVIFLGTLVHKSSSQGQDRHMIRMRQL |
| (forms tetravalent | IDIVDQLKNYVNDLVPEF |
| IL-21 by | LPAPEDVETNCEWSAFSCFQKAQLKSANTGNNERIINVSIKKL |
| disulfide | KRKPPSTNAGRRQKHRL |
| dimerization of | TCPSCDSYEKKPPKEFLERFKSLLQKMIHQHLSSRTHGSEDS |
| Fc regions; SEQ | GGGGGSGGGGSGGGGSGGGGS |
| ID NO: 71) | DKTHTCPPCPAPELLGGPSVFLFPPKPKDTL |
| | MISRTPEVTCVVVDVSHEDPEVKFNWYVD |
| | GVEVHNAKTKPREEQYNSTYRVVSVLTVL |
| | HQDWLNGKEYKCKVSNKALPAPIEKTISKA |
| | KGQPREPQVYTLPPSRDELTKNQVSLTCLVKG |
| | FYPSDIAVEWESNGQPENNYKTTPPVLDSDG |
| | SFFLYSKLTVDKSRWQQGNVFSCSVMHEALHN |
| | HYTQKSLSLSP |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKR |
| | KPPSTNAGRRQKHRL |
| | TCPSCDSYEKKPPKEFLERFKSLLQKMIHQHLSSRTHGSEDS (SEQ |
| | ID NO: 70) |
| TETRAVALENT | MRSSPGNMERIVICLMVIFLGTLVHKSSSQGQDRHMIRMRQL |
| IL-21 | IDIVDQLKNYVNDLVPEF |
| (forms octavalent | LPAPEDVETNCEWSAFSCFQKAQLKSANTGNNERIINVSIKKL |
| IL-21 by | KRKPPSTNAGRRQKHRL |
| disulfide | TCPSCDSYEKKPPKEFLERFKSLLQKMIHQHLSSRTHGSEDS |
| dimerization of | GGGGGSGGGGSGGGGSGGGGS |
| Fc regions; SEQ | HKSSSQGQDRHMIRMRQLIDIVDQLKNYVNDLVPEF |
| ID NO: 73) | LPAPEDVETNCEWSAFSCFQKAQLKSANTGNNERIINVSIKKLKR |
| | KPPSTNAGRRQKHRL |
| | TCPSCDSYEKKPPKEFLERFKSLLQKMIHQHLSSRTHGSEDS |
| | GGGGGSGGGGSGGGGSGGGGS |
| | DKTHTCPPCPAPELLGGPSVFLFPPKPKDTL |
| | MISRTPEVTCVVVDVSHEDPEVKFNWYVD |
| | GVEVHNAKTKPREEQYNSTYRVVSVLTVL |
| | HQDWLNGKEYKCKVSNKALPAPIEKTISKA |
| | KGQPREPQVYTLPPSRDELTKNQVSLTCLVKG |
| | FYPSDIAVEWESNGQPENNYKTTPPVLDSDG |
| | SFFLYSKLTVDKSRWQQGNVFSCSVMHEALHN |
| | HYTQKSLSLSP |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKR |
| | KPPSTNAGRRQKHRL |
| | TCPSCDSYEKKPPKEFLERFKSLLQKMIHQHLSSRTHGSEDS |
| | GGGGGSGGGGSGGGGSGGGGS |
| | HKSSSQGQDRHMIRMRQLIDIVDQLKNYVNDLVPEF |
| | LPAPEDVETNCEWSAFSCFQKAQLKSANTGNNERIINVSIKKLKR |
| | KPPSTNAGRRQKHRL |
| | TCPSCDSYEKKPPKEFLERFKSLLQKMIHQHLSSRTHGSEDS (SEQ |
| | ID NO: 72) |

### Example 6

### Treatment Regimen

A patient presenting with the autoimmune disease systemic lupus erythematosus is started on a regimen of tetravalent IL-2 described herein, administered once daily by intravenous infusion, using a dose level that is identified in a clinical trial to achieve T_{reg} expansion without expanding other, undesirable T cell populations. The patient's T_{reg} abundance increases over time, and resolution of the disease is observed.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A multivalent biomolecule comprising three or more covalently linked common γ-chain receptor cytokines.

2. The multivalent biomolecule of claim 1, wherein the common γ-chain receptor cytokines are IL-2, IL-4, IL-7, IL-9, IL-15 or IL-21, or immunologically active fragments thereof.

3. The multivalent biomolecule of claim 1, comprising at least 3, at least 4, at least 5, at least 6, at least 7, or at least 8 common γ-chain receptor cytokines or immunologically active fragments thereof.

4. The multivalent biomolecule of claim 3, comprising 3, 4, 5, 6, 7 or 8 common γ-chain receptor cytokines or immunologically active fragments thereof.

5. The multivalent biomolecule of claim 1, wherein the common γ-chain receptor cytokines are expressed as Fc fusion proteins of the common γ-chain receptor cytokines or immunologically active fragments thereof with human IgG1 Fc.

6. The multivalent biomolecule of claim 5, wherein the fusion protein comprises the common γ-chain receptor cytokines or immunologically active fragments thereof fused to the N- or C-terminus of human IgG1 Fc.

7. The multivalent biomolecule of claim 6, wherein the common γ-chain receptor cytokines or immunologically active fragments thereof are fused to the N- or C-terminus of human IgG1 Fc through a (G₄S)₄ linker.

8. The multivalent biomolecule of claim 7, wherein the sequence GLNDIFEAQKIEWHE (SEQ ID NO:3) is fused to the terminus of the human IgG1 Fc and fused to the common γ-chain receptor cytokines or immunologically active fragments thereof.

9. The multivalent biomolecule of claim 8, comprising three or more of sequence SEQ ID NO:1, 12, 14, 15, or 26-33.

10. The multivalent biomolecule of claim 1, wherein the common γ-chain receptor cytokines, or an immunologically active fragment thereof, are multimerized and expressed as a single-chain polypeptide.

11. The multivalent biomolecule of claim 10 wherein at least one common γ-chain receptor cytokines, or immunologically active fragments thereof, comprises a signal sequence.

12. The multivalent biomolecule of claim 1, wherein affinity for the cognate γ-chain receptor is at least 2-fold lower, but with equal or greater avidity, than that of the same common γ-chain receptor cytokine in monomeric form.

13. The multivalent biomolecule of claim 1, wherein as compared to biomolecule comprising said γ-chain receptor cytokine in monomeric or dimeric form, said multivalent biomolecule has increased selective binding to cells expressing high level of cognate γ-chain receptor.

14. The multivalent biomolecule of claim 1, wherein the multivalent biomolecule produces an altered dynamic response as compared to a biomolecule comprising said γ-chain receptor cytokine in monomeric or dimeric form, the altered dynamic response is selected from among altered pharmacokinetics, altered intracellular degradation, altered in vivo half-life, or any combination thereof.

15. A method for modulating the immune system of a subject, comprising administering to a subject in need thereof the multivalent biomolecule of claim 1.

16. The method of claim 15, wherein the modulating is activating immune responses in the subject, and the multivalent biomolecule comprises a cytokine selected from IL-2, IL-4, IL-7, IL-9, IL-15 or IL-21.

17. The method of claim 15, wherein the multivalent biomolecule is used for treating cancer.

18. The method of claim 15, wherein the modulating is suppressing immune responses in the subject, and the multivalent biomolecule comprises a cytokine selected from IL-2, IL-4, IL-7, IL-9, IL-15 or IL-21.

19. The method of claim 18, wherein the multivalent biomolecule is used for treating an autoimmune disease or preventing transplant rejection.

20. The method of claim 19, wherein the autoimmune disease is systemic lupus erythematosus.

21. A multivalent cytokine of any one of SEQ ID NOs:2, 4-8, 10, 12, 17-25 or 34-75.

22. A multivalent cytokine comprising a multimer of any one of SEQ ID NOs:9, 10, 58, 60, 11, 74, 62, 64, 66, 68, 70, 72 or 74, or any combination thereof.

23. A pharmaceutical composition comprising a multivalent cytokine of claim 1.

24. A pharmaceutical composition of claim 23 comprising SEQ ID NOs:2, 4-8, 10, 12, 17-25 or 34-75.

25. A pharmaceutical composition of claim 23 wherein the multivalent cytokine comprises a multimer of SEQ ID NOs:9, 10, 58, 60, 11, 74, 62, 64, 66, 68, 70, 72 or 74, or any combination thereof.

26. A nucleic acid encoding a multivalent cytokine of claim 1.

27. A nucleic acid encoding any one of SEQ ID NOs:2, 4-8, 10, 12, 17-22, 34-57, 58, 60, 62, 64, 66, 68, 70, 72 or 74.

28. A vector comprising a nucleic acid encoding a multivalent cytokine of claim 1.

29. A vector comprising a nucleic acid encoding any one of SEQ ID NOs:2, 4-8, 10, 12, 17-22, 34-57, 58, 60, 62, 64, 66, 68, 70, 72 or 74.


SEQUENCE LISTING (76 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 612 aa, Artificial sequence
  Single chain tetravalent IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - protein, 15 aa, Artificial sequence
  AviTag sequence
GLNDIFEAQKIEWHE

SEQ ID NO: 4 - protein, 459 aa, Artificial
  Single chain trivalent IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 5 - protein, 765 aa, Artificial
  Single chain pentavalent IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 6 - protein, 918 aa, Artificial
  Single chain hexavalent IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 1071 aa, Artificial
  Single chain heptavalent IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 1224 aa, Artificial
  Single chain octavalent IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 552 aa, Artificial
  Single chain, bivalent IL-2 with Fc hinge region (SEQ ID NO16)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 858 aa, Artificial
  Single chain, tetravalent IL-2 with Fc hinge region (SEQ ID NO16)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 11 - protein, 595 aa, Artificial
  Single chain, bivalent IL-7 with Fc
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 12 - protein, 133 aa, Homo sapiens [sequence omitted]

SEQ ID NO: 13 - protein, 21 aa, Artificial
  Linker sequence
GGGGGSGGGGSGGGGSGGGGS

SEQ ID NO: 14 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 15 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 16 - protein, 225 aa, Artificial
  Fc hinge region CH2/CH3, without C-terminal K
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 17 - protein, 696 aa, Artificial
  Single chain, tetravalent IL-7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 18 - protein, 869 aa, Artificial
  Single chain, pentavalent IL-7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 19 - protein, 1042 aa, Artificial
  Single chain, hexavalent IL-7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 1215 aa, Artificial
  Single chain, heptavalent IL-7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 21 - protein, 1388 aa, Artificial
  Single chain, octavalent IL-7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 22 - protein, 523 aa, Artificial
  Single chain trivalent IL-7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 23 - protein, 552 aa, Artificial; identical to SEQ ID NO: 9
  Dimer of this sequence linked by disulfides at positions 179 and 182

SEQ ID NO: 24 - protein, 858 aa, Artificial; identical to SEQ ID NO: 10
  Dimer of this sequence linked by disulfides at positions 332 and 335

SEQ ID NO: 25 - protein, 595 aa, Artificial; identical to SEQ ID NO: 11
  Dimer of this sequence linked by disulfides at positions 203 and 206

SEQ ID NO: 26 - protein, 153 aa, Homo sapiens [sequence omitted]

SEQ ID NO: 27 - protein, 129 aa, Homo sapiens [sequence omitted]

SEQ ID NO: 28 - protein, 144 aa, Homo sapiens
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 29 - protein, 126 aa, Homo sapiens
QGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 30 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 31 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 32 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 33 - protein, 138 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 34 - protein, 453 aa, Artificial
  Single chain trivalent IL-4
MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 35 - protein, 603 aa, Artificial
  Single chain tetravalent IL-4
MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 36 - protein, 753 aa, Artificial
  Single chain pentavalent IL-4
MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 37 - protein, 903 aa, Artificial
  Single chain hexavalent IL-4
MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 38 - protein, 1053 aa, Artificial
  Single chain heptavalent IL-4
MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 39 - protein, 1203 aa, Artificial
  Single chain octavalent IL-4
MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 40 - protein, 438 aa, Artificial
  Single chain trivalent IL-9
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 41 - protein, 585 aa, Artificial
  Single chain tetravalent IL-9
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 42 - protein, 732 aa, Artificial
  Single chain pentavalent IL-9
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 43 - protein, 879 aa, Artificial
  Single chain hexavalent IL-9
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 44 - protein, 1026 aa, Artificial
  Single chain heptavalent IL-9
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 45 - protein, 1173 aa, Artificial
  Single chain octavalent IL-9
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 46 - protein, 470 aa, Artificial
  Single chain trivalent IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 47 - protein, 624 aa, Artificial
  Single chain tetravalent IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 48 - protein, 778 aa, Artificial
  Single chain pentavalent IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 49 - protein, 932 aa, Artificial
  Single chain hexavalent IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 50 - protein, 1086 aa, Artificial
  Single chain heptavalent IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 51 - protein, 1240 aa, Artificial
  Single chain octavalent IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 52 - protein, 480 aa, Artificial
  Single chain trivalent IL-21
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 53 - protein, 639 aa, Artificial
  Single chain tetravalent IL-21
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 54 - protein, 798 aa, Artificial
  Single chain pentavalent IL-21
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 55 - protein, 957 aa, Artificial
  Single chain hexavalent IL-21
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 56 - protein, 1116 aa, Artificial
  Single chain heptavalent IL-21
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 57 - protein, 1275 aa, Artificial
  Single chain octavalent IL-21
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 58 - protein, 549 aa, Artificial
  Single chain, bivalent IL-4 with Fc hinge region (SEQ ID NO16)
MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 59 - protein, 549 aa, Artificial; identical to SEQ ID NO: 58
  Dimer of this sequence linked by disulfides at positions 180 and 183

SEQ ID NO: 60 - protein, 849 aa, Artificial
  Single chain, tetravalent IL-2 with Fc hinge region (SEQ ID NO16)
MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGGSGGGGSGGGGSGGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 61 - protein, 849 aa, Artificial; identical to SEQ ID NO: 60
  Dimer of this sequence linked by disulfides at positions 330 and 333

SEQ ID NO: 62 - protein, 537 aa, Artificial
  Single chain, bivalent IL-9 with Fc hinge region (SEQ ID NO16)
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 63 - protein, 537 aa, Artificial; identical to SEQ ID NO: 62
  Dimer of this sequence linked by disulfides at positions 171 and 174

SEQ ID NO: 64 - protein, 831 aa, Artificial
  Single chain, tetravalent IL-9 with Fc hinge region (SEQ ID NO16)
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGGSGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 65 - protein, 831 aa, Artificial; identical to SEQ ID NO: 64
  Dimer of this sequence linked by disulfides at positions 318 and 321

SEQ ID NO: 66 - protein, 562 aa, Artificial
  Single chain, bivalent IL-15 with Fc hinge region (SEQ ID NO16)
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 67 - protein, 562 aa, Artificial; identical to SEQ ID NO: 66
  Dimer of this sequence linked by disulfides at positions 189 and 192

SEQ ID NO: 68 - protein, 870 aa, Artificial
  Single chain, tetravalent IL-15 with Fc hinge region (SEQ ID NO16)
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGGSGGGGSGGGGSGGGGSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 69 - protein, 870 aa, Artificial; identical to SEQ ID NO: 68
  Dimer of this sequence linked by disulfides at positions 343 and 346

SEQ ID NO: 70 - protein, 567 aa, Artificial
  Single chain, bivalent IL-21 with Fc hinge region (SEQ ID NO16)
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 71 - protein, 567 aa, Artificial; identical to SEQ ID NO: 70
  Dimer of this sequence linked by disulfides at positions 189 and 192

SEQ ID NO: 72 - protein, 885 aa, Artificial
  Single chain, tetravalent IL-21 with Fc hinge region (SEQ ID NO16)
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGGSGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 73 - protein, 885 aa, Artificial; identical to SEQ ID NO: 72
  Dimer of this sequence linked by disulfides at positions 348 and 351

SEQ ID NO: 74 - protein, 942 aa, Artificial
  Single chain, tetravalent IL-7 with Fc hinge region (SEQ ID NO16)
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 75 - protein, 942 aa, Artificial; identical to SEQ ID NO: 74
  Dimer of this sequence linked by disulfides at positions 377 and 380

SEQ ID NO: 76 - protein, 138 aa, Artificial
  IL-2 superkine
MGEFAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLTH